SEQUENCE LISTING (33 sequences)

SEQ ID NO: 1 - DNA, 4416 bp, Homo sapiens
atggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggtatctctacaaaacccaccagagtctggcactgatggttgcattttgttaattgttctaagttggtggagcagaactttgcctacttatgtttattgtcaaatgcttctatgcccatttccattccctccataa

SEQ ID NO: 2 - protein, 1471 aa, Homo sapiens
MVSKRRLSKSEDKESLTEDASKTRKQPLSKKTKKSHIANEVEENDSIFVKLLKISGIILKTGESQNQLAVDQIAFQKKLFQTLRRHPSYPKIIEEFVSGLESYIEDEDSFRNCLLSCERLQDEEASMGASYSKSLIKLLLGIDILQPAIIKTLFEKLPEYFFENKNSDEINIPRLIVSQLKWLDRVVDGKDLTTKIMQLISIAPENLQHDIITSLPEILGDSQHADVGKELSDLLIENTSLTVPILDVLSSLRLDPNFLLKVRQLVMDKLSSIRLEDLPVIIKFILHSVTAMDTLEVISELREKLDLQHCVLPSRLQASQVKLKSKGRASSSGNQESSGQSCIILLFDVIKSAIRYEKTISEAWIKAIENTASVSEHKVFDLVMLFIIYSTNTQTKKYIDRVLRNKIRSGCIQEQLLQSTFSVHYLVLKDMCSSILSLAQSLLHSLDQSIISFGSLLYKYAFKFFDTYCQQEVVGALVTHICSGNEAEVDTALDVLLELVVLNPSAMMMNAVFVKGILDYLDNISPQQIRKLFYVLSTLAFSKQNEASSHIQDDMHLVIRKQLSSTVFKYKLIGIIGAVTMAGIMAADRSESPSLTQERANLSDEQCTQVTSLLQLVHSCSEQSPQASALYYDEFANLIQHEKLDPKALEWVGHTICNDFQDAFVVDSCVVPEGDFPFPVKALYGLEEYDTQDGIAINLLPLLFSQDFAKDGGPVTSQESGQKLVSPLCLAPYFRLLRLCVERQHNGNLEEIDGLLDCPIFLTDLEPGEKLESMSAKERSFMCSLIFLTLNWFREIVNAFCQETSPEMKGKVLTRLKHIVELQIILEKYLAVTPDYVPPLGNFDVETLDITPHTVTAISAKIRKKGKIERKQKTDGSKTSSSDTLSEEKNSECDPTPSHRGQLNKEFTGKEEKTSLLLHNSHAFFRELDIEVFSILHCGLVTKFILDTEMHTEATEVVQLGPPELLFLLEDLSQKLESMLTPPIARRVPFLKNKGSRNIGFSHLQQRSAQEIVHCVFQLLTPMCNHLENIHNYFQCLAAENHGVVDGPGVKVQEYHIMSSCYQRLLQIFHGLFAWSGFSQPENQNLLYSALHVLSSRLKQGEHSQPLEELLSQSVHYLQNFHQSIPSFQCALYLIRLLMVILEKSTASAQNKEKIASLARQFLCRVWPSGDKEKSNISNDQLHALLCIYLEHTESILKAIEEIAGVGVPELINSPKDASSSTFPTLTRHTFVVFFRVMMAELEKTVKKIEPGTAADSQQIHEEKLLYWNMAVRDFSILINLIKVFDSHPVLHVCLKYGRLFVEAFLKQCMPLLDFSFRKHREDVLSLLETFQLDTRLLHHLCGHSKIHQDTRLTQHVPLLKKTLELLVCRVKAMLTLNNCREAFWLGNLKNRDLQGEEIKSQNSQESTADESEDDMSSQASKSKATEVSLQNPPESGTDGCILLIVLSWWSRTLPTYVYCQMLLCPFPFPP

SEQ ID NO: 3 - DNA, 4356 bp, Homo sapiens
atggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggatggtgaagaagacgaagtaagtgctggagaaaaggagcaagatagtgatgagagttatgatgactctgattag

SEQ ID NO: 4 - protein, 1451 aa, Homo sapiens
MVSKRRLSKSEDKESLTEDASKTRKQPLSKKTKKSHIANEVEENDSIFVKLLKISGIILKTGESQNQLAVDQIAFQKKLFQTLRRHPSYPKIIEEFVSGLESYIEDEDSFRNCLLSCERLQDEEASMGASYSKSLIKLLLGIDILQPAIIKTLFEKLPEYFFENKNSDEINIPRLIVSQLKWLDRVVDGKDLTTKIMQLISIAPENLQHDIITSLPEILGDSQHADVGKELSDLLIENTSLTVPILDVLSSLRLDPNFLLKVRQLVMDKLSSIRLEDLPVIIKFILHSVTAMDTLEVISELREKLDLQHCVLPSRLQASQVKLKSKGRASSSGNQESSGQSCIILLFDVIKSAIRYEKTISEAWIKAIENTASVSEHKVFDLVMLFIIYSTNTQTKKYIDRVLRNKIRSGCIQEQLLQSTFSVHYLVLKDMCSSILSLAQSLLHSLDQSIISFGSLLYKYAFKFFDTYCQQEVVGALVTHICSGNEAEVDTALDVLLELVVLNPSAMMMNAVFVKGILDYLDNISPQQIRKLFYVLSTLAFSKQNEASSHIQDDMHLVIRKQLSSTVFKYKLIGIIGAVTMAGIMAADRSESPSLTQERANLSDEQCTQVTSLLQLVHSCSEQSPQASALYYDEFANLIQHEKLDPKALEWVGHTICNDFQDAFVVDSCVVPEGDFPFPVKALYGLEEYDTQDGIAINLLPLLFSQDFAKDGGPVTSQESGQKLVSPLCLAPYFRLLRLCVERQHNGNLEEIDGLLDCPIFLTDLEPGEKLESMSAKERSFMCSLIFLTLNWFREIVNAFCQETSPEMKGKVLTRLKHIVELQIILEKYLAVTPDYVPPLGNFDVETLDITPHTVTAISAKIRKKGKIERKQKTDGSKTSSSDTLSEEKNSECDPTPSHRGQLNKEFTGKEEKTSLLLHNSHAFFRELDIEVFSILHCGLVTKFILDTEMHTEATEVVQLGPPELLFLLEDLSQKLESMLTPPIARRVPFLKNKGSRNIGFSHLQQRSAQEIVHCVFQLLTPMCNHLENIHNYFQCLAAENHGVVDGPGVKVQEYHIMSSCYQRLLQIFHGLFAWSGFSQPENQNLLYSALHVLSSRLKQGEHSQPLEELLSQSVHYLQNFHQSIPSFQCALYLIRLLMVILEKSTASAQNKEKIASLARQFLCRVWPSGDKEKSNISNDQLHALLCIYLEHTESILKAIEEIAGVGVPELINSPKDASSSTFPTLTRHTFVVFFRVMMAELEKTVKKIEPGTAADSQQIHEEKLLYWNMAVRDFSILINLIKVFDSHPVLHVCLKYGRLFVEAFLKQCMPLLDFSFRKHREDVLSLLETFQLDTRLLHHLCGHSKIHQDTRLTQHVPLLKKTLELLVCRVKAMLTLNNCREAFWLGNLKNRDLQGEEIKSQNSQESTADESEDDMSSQASKSKATEDGEEDEVSAGEKEQDSDESYDDSD

SEQ ID NO: 5 - DNA, 10257 bp, Homo sapiens
atgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 6 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVAHQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETFYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 7 - DNA, 3750 bp, Homo sapiens
atgtcttcaatgtggtctgaatatacaattggtggggtgaagatttactttccttataaagcttacccgtcacagcttgctatgatgaattctattctcagaggattaaacagcaagcaacattgtttgttggagagtcccacaggaagtggaaaaagcttagccttactttgttctgctttagcatggcaacaatctcttagtgggaaaccagcagatgagggcgtaagtgaaaaagctgaagtacaattgtcatgttgttgtgcatgccattcaaaggattttacaaacaatgacatgaaccaaggaacttcacgtcatttcaactatccaagcacaccaccttctgaaagaaatggcacttcatcaacttgtcaagactcccctgaaaaaaccactctggctgcaaagttatctgctaagaaacaggcatccatatacagagatgaaaatgatgattttcaagtagagaagaaaagaattcgacccttagaaactacacagcagattagaaaacgtcattgctttggaacagaagtacacaatttggatgcaaaagttgattcaggaaagactgtaaaactcaactctccactggaaaagataaactccttttcgccacagaaaccccctggccactgttctaggtgctgttgttctactaaacaaggaaacagtcaagagtcatcgaataccattaagaaggatcatacagggaaatccaagatacccaaaatatattttgggacacgcacacacaagcagattgctcagattactagagagctccggaggacggcatattcaggggttccaatgactattctttccagcagggatcatacttgtgtccatcctgaggtagtcggtaacttcaacagaaatgagaagtgcatggaattgctagatgggaaaaacggaaaatcctgctatttttatcatggagttcataaaattagtgatcagcacacattacagactttccaagggatgtgcaaagcctgggatatagaagaacttgtcagcctggggaagaaactaaaggcctgtccatattacacagcccgagaactaatacaagatgctgacatcatattttgtccctacaactatcttctagatgcacaaataagggaaagtatggatttaaatctgaaagaacaggttgtcattttagatgaagctcataacatcgaggactgtgctcgggaatcagcaagttacagtgtaacagaagttcagcttcggtttgctcgggatgaactagatagtatggtcaacaataatataaggaagaaagatcatgaacccctacgagctgtgtgctgtagcctcattaattggttagaagcaaacgctgaatatcttgtagaaagagattatgaatcagcttgtaaaatatggagtggaaatgaaatgctcttaactttacacaaaatgggtatcaccactgctacttttcccattttgcagggacatttttctgctgttcttcaaaaagaggaaaaaatctcaccaatttatggtaaagaggaggcaagagaagtacctgttattagtgcatcaactcaaataatgcttaaaggactttttatggtacttgactatctttttaggcaaaatagcagatttgcagatgattataaaattgcgattcaacagacttactcctggacaaatcagattgatatttcagacaaaaatgggttgttggttctaccaaaaaataagaaacgttcacgacagaaaactgcagttcatgtgctaaacttttggtgcttaaatccagctgtggccttttcagatattaatggcaaagttcagaccattgttttgacatctggtacattatcaccaatgaaatccttttcgtcagaacttggtgttacatttactatccagctggaggctaatcatatcattaaaaattcacaggtttgggttggtaccattgggtcaggccccaagggtcggaatctctgtgctaccttccagaatactgaaacatttgagttccaagatgaagtgggagcacttttgttatctgtgtgccagactgtgagccaaggaattttgtgtttcttgccatcttacaagttattagaaaaattaaaagaacgttggctctctactggtttatggcataatctggagttggtgaagacagtcattgtagaaccacagggaggagaaaaaacaaattttgatgaattactgcaggtgtactatgacgcaatcaaatacaaaggagagaaagatggagctctcctggtagcagtttgtcgtggtaaagtgagtgagggtctggatttctcagatgacaatgcccgtgctgtcataacaataggaattccttttccaaatgtgaaagatctacaggttgaactaaaacgacaatacaatgaccaccattcaaaattgagaggtcttctacctggccgtcagtggtatgaaattcaagcatacagggccttaaaccaggcccttggtagatgtattagacacagaaatgattggggagctcttattctagtggatgatcgctttaggaataacccaagtcgctatatatctggactttctaaatgggtacggcagcagattcagcaccattcaacctttgaaagtgcactggagtccttggctgaattttccaaaaagcatcaaaaagttcttaatgtatccataaaggacagaaccaatatacaggacaatgagtctacacttgaagtgacctctttaaagtacagtaccccaccttatttactggaagcagcaagtcatctatcaccagaaaattttgtggaagatgaagcaaagatatgtgtccaggaactacagtgtcctaaaattattaccaaaaattcacctctaccaagtagcattatctccagaaaggagaaaaatgatccagtattcctggaagaagcagggaaagcagaaaaaattgtgatttccagatccacaagcccaactttcaacaaacaaacaaagagagttagctggtcaagctttaattctttgggacagtattttactggtaaaataccgaaggcaacacctgagctcgggtcatcagagaatagtgcctctagtcctccccgtttcaaaacagagaagatggaaagtaaaactgttttgcccttcactgataaatgtgaatcctcaaatctgacagtaaacacatcgtttggatcatgccctcaatcagaaaccattatttcatcattaaagattgatgccacccttactagaaaaaatcattctgaacatccgctctgttctgaagaagccctggatccagacattgaattgtctctagtaagtgaagaagataaacagtccacttcaaatagagattttgaaacagaagcagaagatgaatctatctattttacacctgaactttacgatcctgaagatacagatgaagaaaaaaatgacctagctgaaactgatagaggaaatagattggctaacaattcagattgcattttagctaaagacctttttgaaattagaactataaaagaagtagattcagccagagaagtgaaagctgaggattgcatagatacaaagttgaatggaattctgcatattgaagaaagtaaaattgatgacattgatggtaatgtaaaaacaacttggataaatgaactggaactgggaaaaactcatgaaatagaaataaagaactttaaaccatctccttccaaaaataaaggcatgtttcctggttttaagtaa

SEQ ID NO: 8 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTPPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 9 - DNA, 35 bp, Artificial sequence
  Synthetic nucleic acid primer
agctcgagatggtttccaaaagaagactgtcaaaa

SEQ ID NO: 10 - DNA, 35 bp, Artificial sequence
  Synthetic nucleic acid primer
attgcggccgcctaatcagagtcatcataactctc

SEQ ID NO: 11 - DNA, 20 bp, Artificial sequence
  Synthetic nucleic acid primer
caagaaagcagcggtcagag

SEQ ID NO: 12 - DNA, 21 bp, Artificial sequence
  Synthetic nucleic acid primer
acagcaccaataatcccaatg

SEQ ID NO: 13 - DNA, 19 bp, Artificial sequence
  Synthetic nucleic acid primer
agcggtgtggcatcttcac

SEQ ID NO: 14 - DNA, 19 bp, Artificial sequence
  Synthetic nucleic acid primer
gcatgtcgggatggctttc

SEQ ID NO: 15 - DNA, 17 bp, Artificial sequence
  Synthetic nucleic acid probe
caaggcattgtgagcct

SEQ ID NO: 16 - DNA, 21 bp, Artificial sequence
  Synthetic nucleic acid primer
ggaaatcctccagccagagtt

SEQ ID NO: 17 - DNA, 26 bp, Artificial sequence
  Synthetic nucleic acid primer
ggagagaaatcttcttcagcaaaatg

SEQ ID NO: 18 - DNA, 17 bp, Artificial sequence
  Synthetic nucleic acid probe
tgaggctgtaaacgagg

SEQ ID NO: 19 - DNA, 24 bp, Artificial sequence
  Synthetic nucleic acid primer
tgaaatgcacactgaagctacaga

SEQ ID NO: 20 - DNA, 23 bp, Artificial sequence
  Synthetic nucleic acid primer
gagatcttccagcaagaaaagca

SEQ ID NO: 21 - DNA, 16 bp, Artificial sequence
  Synthetic nucleic acid probe
caacttgggccccctg

SEQ ID NO: 22 - DNA, 22 bp, Artificial sequence
  Synthetic nucleic acid primer
tcagcctcagcaatgctactgt

SEQ ID NO: 23 - DNA, 21 bp, Artificial sequence
  Synthetic nucleic acid primer
aaggagaggatccgtccaaga

SEQ ID NO: 24 - DNA, 15 bp, Artificial sequence
  Synthetic nucleic acid probe
ctgaccagaagcctc

SEQ ID NO: 25 - DNA, 17 bp, Artificial sequence
  Synthetic nucleic acid primer
cgtcggccccaagaaga

SEQ ID NO: 26 - DNA, 20 bp, Artificial sequence
  Synthetic nucleic acid primer
tcccctctccaggtgatttg

SEQ ID NO: 27 - DNA, 14 bp, Artificial sequence
  Synthetic nucleic acid probe
tggaacccggcatc

SEQ ID NO: 28 - DNA, 21 bp, Artificial sequence
  Synthetic nucleic acid primer
tgtcctcctgacagcatttgc

SEQ ID NO: 29 - DNA, 21 bp, Artificial sequence
  Synthetic nucleic acid primer
tgtctgggttccctgtgatca

SEQ ID NO: 30 - DNA, 15 bp, Artificial sequence
  Synthetic nucleic acid probe
cgccaaggtctccag

SEQ ID NO: 31 - DNA, 18 bp, Artificial sequence
  Synthetic nucleic acid primer
tgccaagtgcgggactac

SEQ ID NO: 32 - DNA, 22 bp, Artificial sequence
  Synthetic nucleic acid primer
taggcagacacaccagcgtatt

SEQ ID NO: 33 - DNA, 16 bp, Artificial sequence
  Synthetic nucleic acid probe
cacatttcccgggctg

(19) United States
(12) Patent Application Publication
(10) Pub. No.: US 2009/0305266 A1
(43) Pub. Date: Dec. 10, 2009

(54) TEST FOR OVARIAN CANCER BY DETECTING ABNORMALITY IN FANCD2 PATHWAY

(76) Inventors: Grover C. Bagby, Portland, OR (US); Tanja Pejovic, Portland, OR (US); Laura Hays, Portland, OR (US); Susan Olson, Portland, OR (US)

Correspondence Address: KLARQUIST SPARKMAN, LLP, 121 SW SALMON STREET, SUITE 1600, PORTLAND, OR, 97204, US

(21) Appl. No.: 12/299,233

(22) PCT Filed: May 2, 2007
(86) PCT No.: PCT/US07/10762

§ 371 Date: Jan. 14, 2009

Related U.S. Application Data
(60) Provisional application No. 60/797,755, filed on May 3, 2006.

(51) Int. Cl. C12Q 1/68; C12Q 1/02; C40B 30/04; G01N 33/574

(52) U.S. Cl. 435/6; 435/29; 506/9; 435/7.23

(57) ABSTRACT

Methods are provided for determining diagnosing ovarian and breast cancer in a subject, including diagnosing the predisposition of a subject's risk of developing breast or ovarian cancer. The methods include selecting a subject, for example a subject with one or more risk factors for developing ovarian cancer or breast cancer, and detecting a decrease in the activity of the Fanconi anemia (FA) non-nuclear core (NNC) component in the subject. Such a decrease is indicative of a predisposition to ovarian cancer and/or breast cancer in the subject. These methods can be used to monitor the response of a subject to agents designed to prevent breast and ovarian cancer, for example an anti-neoplastic agent. Methods also are provided for identifying agents of use in preventing breast and ovarian cancer.

### CROSS REFERENCE TO RELATED APPLICATION

This application claims the benefit of U.S. Provisional Application No. 60/797,755 filed May 3, 2006, which is incorporated by reference herein in its entirety.

### ACKNOWLEDGMENT OF GOVERNMENT SUPPORT

This work was supported in part by funds from the National Institutes of Health pursuant to grant nos. HL48546 and K512HD043488 and from the Department of Veteran's Affairs Merit Review Award. The Government has certain rights in this invention.

### FIELD

This disclosure relates to the field of diagnostic and prognostic testing for cancer. More specifically this disclosure relates to methods for diagnosing ovarian cancer and breast cancer and predicting the risk of ovarian cancer and breast cancer.

### BACKGROUND

Cancer is the second leading cause of death in the United States, being exceeded by only heart disease. Among women, breast cancer and ovarian cancer are the third and fourth most prevalent cancers, accounting for approximately 34% of newly diagnosed cancers in females.

Although reproductive, demographic, and lifestyle factors affect risk of developing ovarian cancer, the single greatest ovarian cancer risk factor is a family history of the disease. In the United States, 10 to 20 percent of subjects with breast cancer have a first- or second-degree relative with one of these diseases (Madigan et al., J. Natl. Cancer Inst. 87:1681-168, 1995).

The risk of developing ovarian cancer also was found to vary according to the age of diagnosis of the affected relative. In general, the younger the affected relative, the greater the risk to other relatives (Yang et al., Am. J. Epidemiol. 147 (7):652-9, 1998; Colditz et al., JAMA 270 (3):33843, 1993; Slattery and Kerber, JAMA 270 (13): 1563-8, 1993; Pharoah et al., Int. J. Cancer 71 (5):800-9, 1997; Negri et al., Int. J. Cancer 72 (5):735-8, 1997; Hemminki and Vaittinen, Int. J. Cancer 77 (3):386-91, 1998). Other factors contributing to the risk of developing ovarian cancer are the number of affected relatives and the closeness of their biologic relationship (Colditz et al., JAMA 270 (3):338-43, 1993; Slattery and Kerber, JAMA 270 (13):1563-8, 1993; Pharoah et al., Int. J. Cancer 71 (5):800-9, 1997).

Two major genes associated with breast and ovarian cancer have been designated breast cancer susceptibility gene 1 (BRCA1) and breast cancer susceptibility gene 2 (BRCA2) (Miki et al., Science 266:66-71, 1994; Wooster et al., Nature 378:789-792, 1995). While specific mutations in either of these genes confers a lifetime risk of breast cancer of between 60 and 85 percent, and a lifetime risk of ovarian cancer of between 15 and 40 percent (Brose et al., J. Natl. Cancer Inst. 94:1365-1372, 2002, Thompson and Easton, J. Natl. Cancer Inst. 94:1358-136, 52002), mutations in these genes account for a small proportion of all breast cancers (3%) and ovarian cancers (9%) (Newman et al., Epidemiol. Rev. 19:69-79, 1997; Ford etal., Am. J. Hum. Genet. 57:1457-1462, 1995; Offit, Clinical cancer genetics: risk counseling and management. New York: Wiley-Liss, Inc. 1998. 115-124). The majority of breast cancers (97%) and ovarian cancers (91%) are not believed to correlate with BRCA1 and BRCA2 mutations. Therefore the need exist for methods of diagnosing cancer and/or predicting breast cancer risk in subjects that are not believed to harbor BRCA1 and/or BRCA2 mutations.

### SUMMARY

The present disclosure relates to the discovery that the Fanconi anemia (FA) non-nuclear core complex (NNC) component is involved in ovarian carcinogenesis. Accordingly, methods are disclosed for diagnosing ovarian and/or breast cancer in a subject, for example diagnosing existing ovarian and/or breast cancer in a subject or diagnosing a predisposition to developing ovarian and/or breast cancer. In some embodiments, the methods for diagnosing ovarian and/or breast cancer in a subject include detecting a decrease in the activity of the FA NNC component in tissue obtained from a subject. In certain examples, tissue-specific suppression of the activity of the FA NNC component indicates that a subject has ovarian and/or breast cancer or is at substantial risk for developing breast and/or ovarian cancer. In particular examples, determining a decrease in the activity of the FA NNC component can be determined by detecting decreased expression of one or more of the FA NNC component members such as FANCD2, FANCD1, or FANCJ, for example decreased expression of FANCD2, FANCD1, and FANCJ, decreased expression of FANCD2 and FANCJ, or even decreased expression of FANCD2.

In some embodiments, the decrease in activity of the FA NNC component is determined by detecting an increase in chromosomal breakage and/or radial formation in response to a DNA damaging agent. In some examples, at least one cell (for example one or more isolated cells, such as cells of female reproductive tissue from a subject) is provided. The cells of the female reproductive tissue are contacted with at least one DNA damaging agent (for example a DNA crosslinking agent) and chromosomal breakage and radial formation is detected in the cell(s). An increase in one or more of chromosomal breakage and radial formation (for example relative to a control) indicates a subject has ovarian and/or breast cancer or is predisposed to developing ovarian and/or breast cancer. Examples of suitable crosslinking agents for use in the disclosed methods include alkylating agents, for example mitomycin C (MMC) and diepoxybutane (DEB), although any agent that produces crosslinks in sufficient quantity can be used.

In some embodiments, detecting a decrease in the activity of the FA NNC component is made in comparison to a control. Examples of controls of use in the disclosed methods include immortalized ovarian epithelial cells, ovarian cells obtained from subjects that do not have ovarian cancer, cells from subjects that do not have any known risk factors for ovarian and/or breast cancer, cells from ovarian tissue from the subject at an earlier time point (for example, prior to onset of ovarian cancer) non-reproductive tissue obtained from the subject, for example blood cells, such as leukocytes, for example lymphocytes, or statistical controls. In some examples, the control is a standard level of chromosomal breakage established from the type of cells. In some examples, the control is a standard level of expression members of the FA NNC component established from such cells.

In certain embodiments of the methods disclosed herein, a decrease in activity of one or more members of the FA NNC component is identified in a subject's tissue, for example in tissue obtained from the subject, such as tissue isolated from the subject. Examples of tissue that can be used with the disclosed methods include female reproductive tissue, such as breast tissue, ovarian tissue, ovarian epithelial tissue, cervical tissue, and cervical epithelial tissue.

In some embodiments, a subject is selected based on the presence of ovarian or breast cancer risk factors. Examples of ovarian and/or breast cancer risk factors include without limitation a prior diagnosis of existing ovarian and/or breast cancer in the subject, and/or a family history of the prior occurrence of one or more of ovarian and/or breast cancer. In some examples, a family history of breast cancer includes a prior diagnosis of existing ovarian and/or breast cancer in one or more 1st degree relatives, prior diagnosis of existing ovarian and/or breast cancer in one or more 1st degree relatives before age 50, prior diagnosis of existing ovarian and/or breast cancer in one or more 1st degree relatives and prior diagnosis of existing breast or ovarian cancer in one or more 1st or 2nd degree relatives, and prior diagnosis of existing breast or ovarian cancer in the subject and prior diagnosis of existing breast or ovarian cancer in one or more 1st or 2nd degree relatives. It will be appreciated that other risk factors can also be used to select a subject. In some examples of the disclosed methods, subjects are selected that do not have any known risk factor for breast or ovarian cancer, for example as preventative screening, or where the breast and or ovarian cancer status of relatives is unknown.

This disclosure further relates to the use of the methods disclosed herein to monitor a response to a treatment for ovarian and/or breast cancer. For example, a drug (such as an anti-neoplastic agent or other treatment) is administered to a subject, a sample of female reproductive tissue is obtained from the subject, and the tissue sample analyzed for activity of the FA NNC component. Increased activity of the FA NNC component indicates that the treatment is effective in treating breast and/or ovarian cancer in the subject.

This disclosure further relates to methods for identifying an agent that inhibits ovarian and/or breast cancer. For example, a cell, such as an ovarian cancer cell, is contacted with a test agent and the activity of the FA NNC component determined. An increase in activity of the FA NNC component in the cell indicates an agent that can be selected for further study and characterization, for example as a potential treatment for breast and/or ovarian cancer. It will be understood that any technique or method can be used to measure the activity of the FA NNC component.

The foregoing and other objects, features, and advantages of the invention will become apparent from the following detailed description, which proceeds with reference to the accompanying figures.

### BRIEF DESCRIPTION OF THE DRAWINGS

FIG. 1A-1D is a set of digital images of western blots showing FANCD2 expression but not FANCA or FANCC is reduced in ovarian high-risk epithelium and ovarian cancer. Primary ovarian epithelial cells were treated with 50 nM MMC for 48 hours before harvest. FIG. 1A is a digital image of an immunoblot demonstrating that immunoblotting of cell extracts with anti-FANCD2 mouse monoclonal antibody shows differentially expressed FANCD2 protein bands in normal ovarian epithelium (OV-NL9), versus high-risk OSE (OV-HR2) and ovarian cancer (OV-CA4). FANCD2-S and FANCD2-L bands are indicated. In high-risk ovarian epithelium and ovarian cancer cells, the overall level of FANCD2 was reduced. FIG. 1B and FIG. 1C are digital images of immunoblots with anti-FANCA antibody and anti-FANCC antibody, respectively. In high-risk ovarian epithelium and ovarian cancer cells there was no down regulation of expression of either FANCA or FANCC. Alpha-tubulin loading controls are shown beneath each panel. FIG. 1D is a digital image of and immunoblot with anti-p53 antibody. In high-risk ovarian epithelium and ovarian cancer cells there was a slight down regulation of expression of the tumor suppressor p53. Alpha-tubulin loading controls are shown.

FIGS. 2A and 2B are a bar graph and digital images of a set of immunoblots showing relative amounts of FANCD2 mRNA and protein in peripheral blood mononuclear lymphocytes (PBML) and ovarian surface epithelial cells (OSE) obtained from a high-risk (OV-HR2) and ovarian cancer subject (OV-CA4). FIG. 2A is a bar graph illustrating relative amounts of FANCD2 RNA, reverse transcribed, and FANCD2 cDNA, amplified by TAQMAN® real-time PCR. “Normal” bars represent mean expression levels in two normal individuals. Triplicate measurements were taken for each sample shown. FIG. 2B is a digital image of an immunoblot illustrates FANCD2 protein expression, where the lower panel shows PBML from two normal controls (Nl9, Nl10), a high-risk subject (HR2), and ovarian cancer subject (CA4). Cells were PHA-stimulated for 96 hours. Nl10 is a healthy volunteer, with no associated ovarian or other pathology. PBML protein lysates were immunoblotted for FANCD2 protein. Ponceau S stain for total protein is shown below each lane. The top panel shows FANCD2 expression in normal ovarian epithelial cells (OV-Nl9 and OV-Nl3), high-risk OSE (OV-HR2), and ovarian cancer (OV-CA4). Cells were treated with 50 nM MMC for 48 hours before harvest. In both OV-H2 and OV-CA4, FANCD2-L and FANCD2-S forms are apparent with longer exposures.

FIG. 3 is a digital image of a western blot demonstrating the restoration of FANCD2 expression after retroviral transduction with normal FANCD2 cDNA. SV40-transformed ovarian epithelial high-risk cells (OV-HR2) and ovarian cancer cells (OV-CA4) were transduced with pMMP retroviral vectors containing full length FANCD2 cDNA. Ovarian epithelial cells were treated with 50 nM MMC for 48 hours before harvest. Immunoblotting of cell extracts with anti-FANCD2 mouse monoclonal antibody shows FANCD2 protein bands in normal ovarian epithelium (OV-Nl9), high-risk OSE (OV-HR2), and ovarian cancer (OV-CA4). FANCD2-S and FANCD2-L bands are indicated. Alpha-tubulin was used as a protein loading control.

FIGS. 4A and 4B are a set of graphs demonstrating that FANCD2 complementation 30 increases survival of high-risk and malignant ovarian epithelial cells, as well as partially correcting MMC-induced radial formation. FIG. 4A is a line graph showing cell survival, measured by the trypan blue dye exclusion method (closed symbols), of OV-HR2 and OV-CA4. Cells were SV40-transformed and treated for 5 days with MMC (0-250 nM). Cell viability was expressed as percent of trypan blue-excluding cells in the MMC-treated sample relative to a corresponding untreated control sample. The same cell survival assay was performed on SV40-transformed OV-HR2 and OV-CA4 after retroviral transduction with normal FANCD2 cDNA (open symbols). Data points represent the mean ±S.D. of three biological replicates. FIG. 4B is a bar graph showing that FANCD2 complementation partially corrects MMC-induced radial formation in high-risk (OV-HR2) and malignant (OV-CA4) ovarian epithelium. FANCD2-deficient (“WT”) and FANCD2-complemented (“WT/D2”) SV40-transformed OV-HR2 and OV-CA4 ovarian epithelial cells were incubated with 40 ng/ml MMC or 200 ng/mL of DEB for 48 hours. The cultures were harvested after a 2 hour treatment with 0.25 μg/ml colcemid and stained with Wright's stain. The percent of radial formations out of 50 metaphases examined per case was determined. A cut-off value of 20% was used to distinguish normal versus increased radial formation.

FIG. 5 is a representative photomicrograph of metaphase chromosomes from a high-risk subject showing chromosome radials and breaks in response to MMC.

FIG. 6 is a schematic representation of the Fanconi anemia pathway. The Fanconi anemia pathway includes two distinct components. The first is the FA nuclear core complex and the second is the FA non-nuclear core complex (NNC). The nuclear core complex detects DNA damage, for example induced by MMC and DEB, and signals the activation of the FA NNC via monoubiquitination of the FANCD2 protein. Abbreviations: A, FANCA; B, FANCB; C, FANCC; D1, FANCD1; D2, FANCD2; E, FANCE; F, FANCF; G, FANCG; J, FANCJ; L, FANCL; M, FANCM.

FIG. 7A-7E is a series of capillary electrophoresis (CE) traces showing the methylation status analysis of the FA gene promoters. Only part of the CE pattern is displayed. The two sets of signals correspond with the amplified probes of HhaI-undigested and HhaI-digested samples, respectively. The probe names representing the gene promoters are shown on top of the peak signals. Control probes that do not contain an HhaI recognition sequence are marked with a “c”. Probes designed to detect CpG methylation are marked with an “m”. FIG. 7A shows the CE pattern of a wild-type DNA sample with no methylation of any of the FA gene promoters. Note that only the control probes display an amplification product after HhaI restriction enzyme treatment. FIG. 7B shows the CE pattern of an SssI methyltransferase-treated wild-type DNA sample. CpG methylated sequences result in the amplification of all the “m” probes. FIG. 7C, FIG. 7D, and FIG. 7E show the CE patterns of the OV-CA4, OV-HR2, and OV-NL9 samples, respectively. No methylation of any of the FA gene promoters was observed.

FIG. 8A-8D are a set of bar graphs showing the proliferation state of the indicated ovarian cell cultures. Real-time PCR was performed on triplicate 50 ng cDNA samples. Probes for proliferating cell nuclear antigen (PCNA, assay ID Hs00427214_gl) and cyclin D1 (assay ID Hs00277039_ml) were purchased as TAQMAN® Gene Expression assays from Applied Biosystems. All reactions were performed in multiplex format with a VIC-labeled 18S rRNA probe. FIG. 8A and FIG. 8B show the relative mRNA levels of cyclin D1 and PCNA respectively in primary ovarian epithelial cultures. FIG. 8C and FIG. 8D show the relative mRNA levels of cyclin D1 and PCNA respectively in SV40-transformed ovarian epithelial cultures, and the same cultures transduced with FANCD2. No pattern distinguished normal primary OSE from cancer/high-risk OSE, or SV40-transformed OSE from their FANCD2-expressing counterparts.

FIG. 9A-9C FANCD2ex16-18del cDNA is nonfunctional. FIG. 9A, gel electrophoresis of RT-PCR products from normal PBML (lane 1) or normal ovarian epithelium (lane 2). Amplification controls were a plasmid template containing the wild-type FANCD2 cDNA (lane 3) or a plasmid template containing the cloned FANCD2ex16-18del splice form (lane 4). Right, exon structure of highlighted PCR products. Arrows, PCR primer binding sites. Numbered boxes, exons; number above boxes, predicted amplimer size. FIG. 9B, PD20 human fibroblasts were transduced with a pLXSN retrovirus expressing either wild-type FANCD2 or the FANCD2ex16-18del form. Cell survival was measured by trypan blue exclusion. FIG. 9C, JY normal human lymphoblasts were transduced with the FANCD2ex16-18del retrovirus, and cell survival

### SEQUENCE LISTING

The nucleic and amino acid sequences listed in the accompanying sequence listing are shown using standard letter abbreviations for nucleotide bases, and one letter code for amino acids, as defined in 37 C.F.R. 1.822. Only one strand of each nucleic acid sequence is shown, but the complementary strand is understood as included by any reference to the displayed strand.
SEQ ID NO: 1 is an exemplary nucleotide sequence of splice variant 1 of human FANCD2.
SEQ ID NO: 2 is an exemplary amino acid sequence of splice variant 1 of human FANCD2.
SEQ ID NO: 3 is an exemplary nucleotide sequence of splice variant 2 of human FANCD2.
SEQ ID NO: 4 is an exemplary amino acid sequence of splice variant 2 of human FANCD2.
SEQ ID NO: 5 is an exemplary nucleotide sequence of human FANCD1.
SEQ ID NO: 6 is an exemplary amino acid sequence of human FANCD1.
SEQ ID NO: 7 is an exemplary nucleotide sequence of human FANCJ.
SEQ ID NO: 8 is an exemplary amino acid sequence of human FANCJ.
SEQ ID NO: 9 is the nucleotide sequence of primer Xho-D2-1.
SEQ ID NO: 10 is the nucleotide sequence of primer Not-D2-441.
SEQ ID NO: 11 is the nucleotide sequence of a FANCD2 upstream sequencing primer.
SEQ ID NO: 12 is the nucleotide sequence of a FANCD2 downstream sequencing primer.
SEQ ID NO: 13 is the nucleotide sequence of a FANCA forward real time RT-PCR primer.
SEQ ID NO: 14 is the nucleotide sequence of a FANCA reverse real time RT-PCR primer.
SEQ ID NO: 15 is the nucleotide sequence of a FANCA real time RT-PCR probe.
SEQ ID NO: 16 is the nucleotide sequence of a FANCC forward real time RT-PCR primer.
SEQ ID NO: 17 is the nucleotide sequence of a FANCC reverse real time RT-PCR primer.
SEQ ID NO: 18 is the nucleotide sequence of a FANCC real time RT-PCR probe.
SEQ ID NO: 19 is the nucleotide sequence of a FANCD2 forward real time RT-PCR primer.
SEQ ID NO: 20 is the nucleotide sequence of a FANCD2 reverse real time RT-PCR primer.
SEQ ID NO: 21 is the nucleotide sequence of a FANCD2 real time RT-PCR probe.
SEQ ID NO: 22 is the nucleotide sequence of a FANCE forward real time RT-PCR primer.
SEQ ID NO: 23 is the nucleotide sequence of a FANCE reverse real time RT-PCR primer.
SEQ ID NO: 24 is the nucleotide sequence of a FANCE real time RT-PCR probe.
SEQ ID NO: 25 is the nucleotide sequence of a FANCF forward real time RT-PCR primer.
SEQ ID NO: 26 is the nucleotide sequence of a FANCF reverse real time RT-PCR primer.
SEQ ID NO: 27 is the nucleotide sequence of a FANCF real time RT-PCR probe.
SEQ ID NO: 28 is the nucleotide sequence of a FANCG forward real time RT-PCR primer.
SEQ ID NO: 29 is the nucleotide sequence of a FANCG reverse real time RT-PCR primer.
SEQ ID NO: 30 is the nucleotide sequence of a FANCG real time RT-PCR probe.
SEQ ID NO: 31 is the nucleotide sequence of a FANCM forward real time RT-PCR primer.
SEQ ID NO: 32 is the nucleotide sequence of a FANCM reverse real time RT-PCR primer.
SEQ ID NO: 33 is the nucleotide sequence of a FANCM real time RT-PCR probe.

### DETAILED DESCRIPTION

### I. Abbreviations

cDNA: Complementary DNA

Cₜ: Cycle threshold

DEB: Diepoxybutane

DMEM: Dulbecco's modified eagle medium

EDTA: Ethylenediaminetetraacetic acid

ELISA: enzyme-linked immunosorbent assay

FACS: Fluorescence activated cell sorting

FCS: Fetal calf serum

HEPES: 4-(2-hydroxyethyl)-1-piperazineethanesulfonic acid

LOV: left ovary

MMC: Mitomycin C

MS-MLPA: Methylation-Specific MLPA

OSE: Ovarian surface epithelium

PBML: Peripheral blood mono-lymphocytes

PBS: Phospho buffered saline

PCR: Polymerase chain reaction

RT-PCR: Reverse transcriptase PCR

SDS: Sodium dodecyl (lauryl) sulfate

SDS-PAGE: Sodium dodecyl (lauryl) sulfate-polyacrylamide gel electrophoreses

SV 40: Simian Virus 40

TBS-T: Tris-Buffered Saline Tween-20

TRIS: Tris-hydroxymethylaminoethane

RIA: Radioimmunoassays

ROV: Right ovary

To facilitate review of the various embodiments of this disclosure, the following explanations of specific terms are provided:

### II. Terms

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Activity of the FA NNC component: describes the ability of cells to protect cells against DNA damage via the FA NNC component. A decrease in the activity of the FA NNC component leads to an inability of cells to repair DNA damage, such as that induced by a DNA crosslinking agent. Decreased expression of one or more of the members of FA NNC component (for example FANCD1, FANCD2, or FANCJ) decreases activity of the FA NNC component.

Antibody: A polypeptide ligand comprising at least a light chain or heavy chain immunoglobulin variable region, which specifically recognizes and binds an epitope of an antigen, such as a FANCD2, FANCD1, or FANCJ protein or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Cancer: A malignant disease characterized by the abnormal growth and differentiation of cells. “Metastatic disease” refers to cancer cells that have left the original tumor site and migrate to other parts of the body for example via the bloodstream or lymph system. “Gynecological cancers” include cancers of the uterus (for example endometrial carcinoma), cervix (for example cervical carcinoma, pre-tumor cervical dysplasia), ovaries (for example ovarian carcinoma, serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, malignant teratoma), vulva (for example squamous cell carcinoma, intraepithelial carcinoma, adenocarcinoma, fibrosarcoma, melanoma), vagina (for example clear cell carcinoma, squamous cell carcinoma, botryoid sarcoma), embryonal rhabdomyosarcoma, and fallopian tubes (for example carcinoma). “Breast cancer” includes cancers of the breast tissue, such as adenocarcinoma. The most common type of breast cancer is ductal carcinoma. Ductal carcinoma in situ is a non-invasive neoplastic condition of the ducts. Lobular carcinoma is not an invasive disease, but it is an indicator that a carcinoma may develop.

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating a breast cancer, an ovarian cancer, or another tumor, such as an anti-neoplastic agent. In one embodiment, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995, Fischer Knobf, and Durivage (eds): The Cancer Chemotherapy Handbook, 4th ed. St. Louis, Mosby-Year Book, 1993). Chemotherapeutic agents used for treating breast and ovarian cancer include cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone and vinorelbine. Combination chemotherapy is the administration of more than one agent to treat cancer.

Chromosome: A chromosome is a very long continuous piece of DNA, containing many genes, regulatory elements, and other intervening nucleotide sequences. During cell division, chromosomes become highly condensed distinct bodies within the nuclei of cells. During the metaphase stage of cell division chromosomes are most easily visualized by techniques such as by staining metaphase spreads and the use of light microscopy. A chromosome has exactly one centromere. During metaphase, following replication, the chromosome appears as two sister chromatids joined at the centromere. “Chromosomal breakage” describes a phenomenon in which the chromosomes of a subject have broken into smaller fragments. Typically, breaks are achromatic areas greater than one chromatid in width. “Radial formation” is the joining of two or more chromosomes or chromosomal fragments to form a super chromosomal structure. Radial formations are so named for their spoke like appearance. Typical radials are designated as tri-radials, quadra-radials, etc depending on the number of arms.

Contacting: Placement in direct physical association. Includes both in solid and liquid form. Contacting can occur in vitro with isolated cells or in vivo by administering to a subject.

Control: A reference standard. A control can be a known value indicative basal expression of FANCD2, FANCD1, and/or FANCJ, for example in a normal cell or a cell not contacted with an agent. In other examples, the control is a standard level of chromosomal breakage established from such cells.

A difference between a test sample and a control can be an increase or conversely a decrease. The difference can be a qualitative difference or a quantitative difference, for example a statistically significant difference. In some examples, a difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

Corresponding: The term “corresponding” is a relative term indicating similarity in position, purpose, or structure.

Detect: To determine if an agent (such as a signal or particular nucleotide nucleic acid probe, amino acid, or protein, for example a FANCD2, FANCD1, or FANCJ protein or nucleic acid) is present or absent. In some examples, this can further include quantification.

Determining expression of a gene product: Detection of a level of expression (for example protein or nucleic acid) in either a qualitative or a quantitative manner. In one example, it is the detection of a FANCD2 gene product. In another example, it is the detection of a FANCD1 gene product. In yet another example, it is the detection of a FANCJ gene product.

Diagnosis: The process of identifying a disease or a predisposition to developing a disease, such as breast or ovarian cancer, by its signs, symptoms, and results of various tests and methods, for example the methods disclosed herein. The conclusion reached through that process is also called “a diagnosis.” Forms of testing commonly performed include blood tests, medical imaging, urinalysis, PAP smear, and biopsy. The term “predisposition” refers to an effect of a factor or factors that render a subject susceptible to a condition, disease, or disorder, such as cancer, for example by a reduction in the activity of the FA NNC component. In some examples, of the disclosed methods, testing is able to identify a subject predisposed to developing a condition, disease, or disorder, such as breast and/or ovarian cancer.

Downregulated or inactivated: When used in reference to the expression of a gene product such as a nucleic acid molecule, for example a gene, or a protein it refers to any process which results in a decrease in production of the gene product. A gene product can be a DNA, an RNA (such as mRNA, rRNA, tRNA, and structural RNA), or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription, and those that increase transcriptional repression. Gene downregulation can include reduction of expression above an existing level. Examples of processes that decrease translation include those that decrease translational initiation, those that decrease translational elongation, and those that decrease mRNA stability.

Gene downregulation includes any detectable decrease in the production of a gene product. In certain examples, production of a gene product, for example a FANCD2, FANCD1, FANCJ gene product, decreases by at least 2-fold, for example at least 3-fold, at least 4-fold, at least 5-fold, at least 6-fold, or more as compared to a control (such an amount of gene expression in a normal cell).

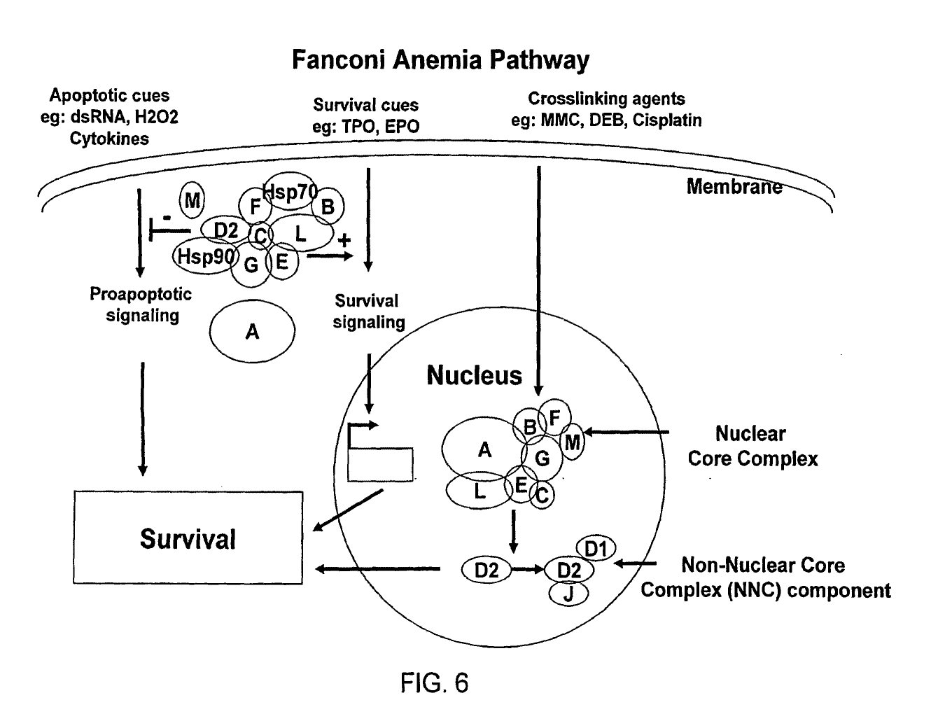

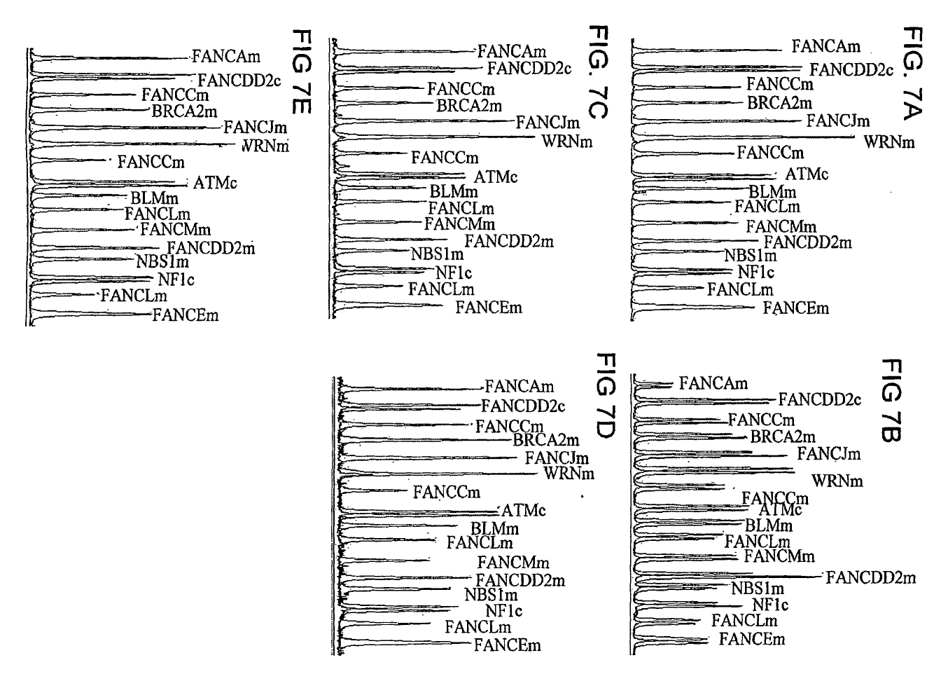

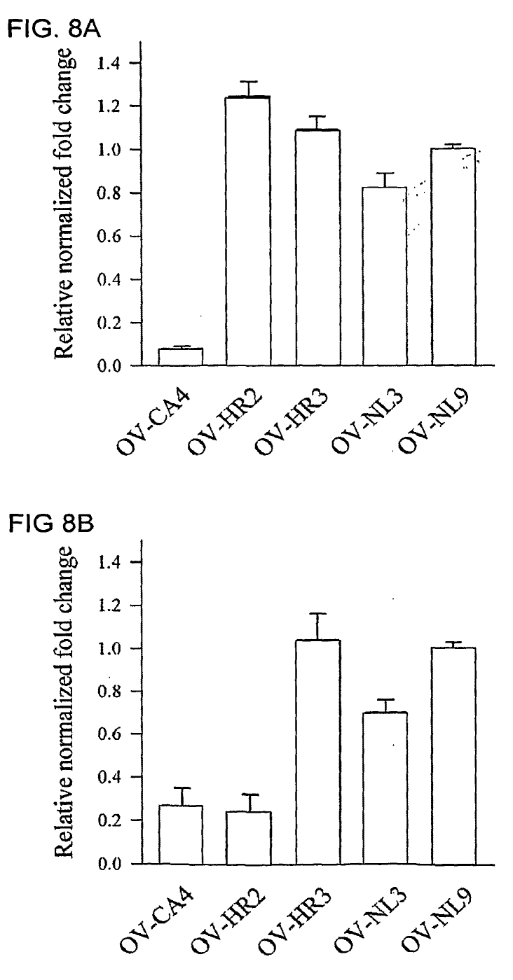

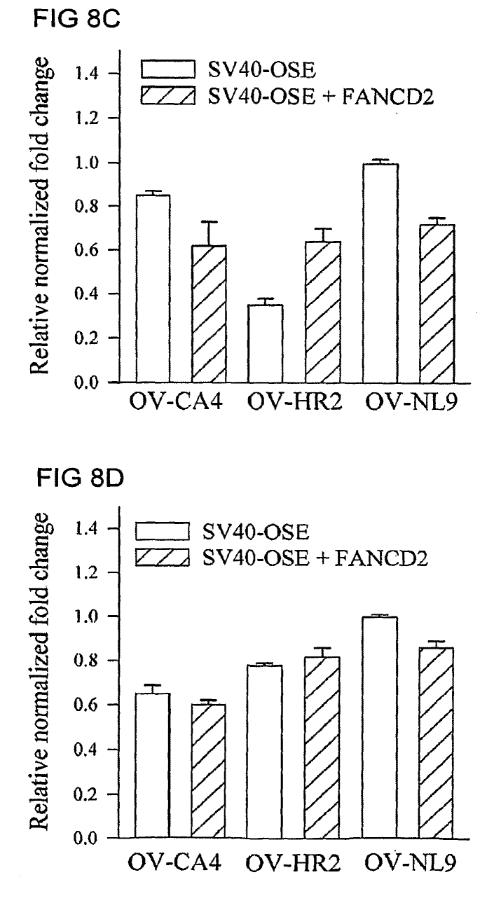

Fanconi anemia pathway: The “Fanconi Anemia Pathway” refers to the functional relationship that exists between nine of the eleven FA proteins (FANCA, -B, -C, -D2, -E, -F, -G, -L, and -M) in nuclear responses to DNA cross-links. With reference to FIG. 6, eight of these proteins (FANCA, FANCB, FANCC, FANCE, FANCF, FANCG, FANCL, and FANCM) form a complex that localizes to the nucleus and is termed the FA nuclear core complex. The remaining three proteins (FANCD2, FANCD1, and FANCJ) are collectively referred to as the FA non-nuclear core (NNC) component. Disruption of activity of the FA NNC component leads to an inability of cells to repair DNA damage, such as that induced by a DNA crosslinking agent.

FANCD2: A member of the FA NNC component identified from the Fanconi anemia complementation group D. In normal cells, FANCD2 protein is activated by monoubiquitination and/or phosphorylation in response to DNA damage. Exemplary nucleotide sequences of FANCD2 as found at GENBANK® accession number NM_{—}033084 (as available Nov. 18, 2006) and NM_{—}001018115 (as available Mar. 1, 2007) are shown below. Also shown are the amino acid sequences encoded by these nucleotide sequences.

FANCD2 SPLICE VARIANT 1 (NM_033084)

(SEQ ID NO: 1)

atggtttccaaaagaagactgtcaaaatctgaggataaagagagcctgac
agaagatgcctccaaaaccaggaagcaaccactttccaaaaagacaaaga
aatctcatattgctaatgaagttgaagaaaatgacagcatctttgtaaag
cttcttaagatatcaggaattattcttaaaacgggagagagtcagaatca
actagctgtggatcaaatagctttccaaaagaagctctttcagaccctga
ggagacacccttcctatcccaaaataatagaagaatttgttagtggcctg
gagtcttacattgaggatgaagacagtttcaggaactgccttttgtcttg
tgagcgtctgcaggatgaggaagccagtatgggtgcatcttattctaaga
gtctcatcaaactgcttctggggattgacatactgcagcctgccattatc
aaaaccttatttgagaagttgccagaatatttttttgaaaacaagaacag
tgatgaaatcaacatacctcgactcattgtcagtcaactaaaatggcttg
acagagttgtggatggcaaggacctcaccaccaagatcatgcagctgatc
agtattgctccagagaacctgcagcatgacatcatcaccagcctacctga
gatcctaggggattcccagcacgctgatgtggggaaagaactcagtgacc
tactgatagagaatacttcactcactgtcccaatcctggatgtcctttca
agcctccgacttgacccaaacttcctattgaaggttcgccagttggtgat
ggataagttgtcgtctattagattggaggatttacctgtgataataaagt
tcattcttcattccgtaacagccatggatacacttgaggtaatttctgag
cttcgggagaagttggatctgcagcattgtgttttgccatcacggttaca
ggcttcccaagtaaagttgaaaagtaaaggacgagcaagttcctcaggaa
atcaagaaagcagcggtcagagctgtattattctcctctttgatgtaata
aagtcagctattagatatgagaaaaccatttcagaagcctggattaaggc
aattgaaaacactgcctcagtatctgaacacaaggtgtttgacctggtga
tgcttttcatcatctatagcaccaatactcagacaaagaagtacattgac
agggtgctaagaaataagattcgatcaggctgcattcaagaacagctgct
ccagagtacattctctgttcattacttagttcttaaggatatgtgttcat
ccattctgtcgctggctcagagtttgcttcactctctagaccagagtata
atttcatttggcagtctcctatacaaatatgcatttaagttttttgacac
gtactgccagcaggaagtggttggtgccttagtgacccatatctgcagtg
ggaatgaagctgaagttgatactgccttagatgtccttctagagttggta
gtgttaaacccatctgctatgatgatgaatgctgtctttgtaaagggcat
tttagattatctggataacatatcccctcagcaaatacgaaaactcttct
atgttctcagcacactggcatttagcaaacagaatgaagccagcagccac
atccaggatgacatgcacttggtgataagaaagcagctctctagcaccgt
attcaagtacaagctcattgggattattggtgctgtgaccatggctggca
tcatggcggcagacagaagtgaatcacctagtttgacccaagagagagcc
aacctgagcgatgagcagtgcacacaggtgacctccttgttgcagttggt
tcattcctgcagtgagcagtctcctcaggcctctgcactttactatgatg
aatttgccaacctgatccaacatgaaaagctggatccaaaagccctggaa
tgggttgggcataccatctgtaatgatttccaggatgccttcgtagtgga
ctcctgtgttgttccggaaggtgactttccatttcctgtgaaagcactgt
acggactggaagaatacgacactcaggatgggattgccataaacctcctg
ccgctgctgttttctcaggactttgcaaaagatgggggtccggtgacctc
acaggaatcaggccaaaaattggtgtctccgctgtgcctggctccgtatt
tccggttactgagactttgtgtggagagacagcataacggaaacttggag
gagattgatggtctactagattgtcctatattcctaactgacctggagcc
tggagagaagttggagtccatgtctgctaaagagcgttcattcatgtgtt
ctctcatatttcttactctcaactggttccgagagattgtaaatgccttc
tgccaggaaacatcacctgagatgaaggggaaggtgctcactcggttaaa
gcacattgtagaattgcaaataatcctggaaaagtacttggcagtcaccc
cagactatgtccctcctcttggaaactttgatgtggaaactttagatata
acacctcatactgttactgctatttcagcaaaaatcagaaagaaaggaaa
aatagaaaggaaacaaaaaacagatggcagcaagacatcctcctctgaca
cactttcagaagagaaaaattcagaatgtgaccctacgccatctcataga
ggccagctaaacaaggagttcacagggaaggaagaaaagacatcattgtt
actacataattcccatgcttttttccgagagctggacattgaggtcttct
ctattctacattgtggacttgtgacgaagttcatcttagatactgaaatg
cacactgaagctacagaagttgtgcaacttgggccccctgagctgctttt
cttgctggaagatctctcccagaagctggagagtatgctgacacctccta
ttgccaggagagtcccctttctcaagaacaaaggaagccggaatattgga
ttctcacatctccaacagagatctgcccaagaaattgttcattgtgtttt
tcaactgctgaccccaatgtgtaaccacctggagaacattcacaactatt
ttcagtgtttagctgctgagaatcacggtgtagttgatggaccaggagtg
aaagttcaggagtaccacataatgtcttcctgctatcagaggctgctgca
gatttttcatgggctttttgcttggagtggattttctcaacctgaaaatc
agaatttactgtattcagccctccatgtccttagtagccgactgaaacag
ggagaacacagccagcctttggaggaactactcagccagagcgtccatta
cttgcagaatttccatcaaagcattcccagtttccagtgtgctctttatc
tcatcagacttttgatggttattttggagaaatcaacagcttctgctcag
aacaaagaaaaaattgcttcccttgccagacaattcctctgtcgggtgtg
gccaagtggggataaagagaagagcaacatctctaatgaccagctccatg
ctctgctctgtatctacctggagcacacagagagcattctgaaggccata
gaggagattgctggtgttggtgtcccagaactgatcaactctcctaaaga
tgcatcttcctccacattccctacactgaccaggcatacttttgttgttt
tcttccgtgtgatgatggctgaactagagaagacggtgaaaaaaattgag
cctggcacagcagcagactcgcagcagattcatgaagagaaactcctcta
ctggaacatggctgttcgagacttcagtatcctcatcaacttgataaagg
tatttgatagtcatcctgttctgcatgtatgtttgaagtatgggcgtctc
tttgtggaagcatttctgaagcaatgtatgccgctcctagacttcagttt
tagaaaacaccgggaagatgttctgagcttactggaaaccttccagttgg
acacaaggctgcttcatcacctgtgtgggcattccaagattcaccaggac
acgagactcacccaacatgtgcctctgctcaaaaagaccctggaactttt
agtttgcagagtcaaagctatgctcactctcaacaattgtagagaggctt
tctggctgggcaatctaaaaaaccgggacttgcagggtgaagagattaag
tcccaaaattcccaggagagcacagcagatgagagtgaggatgacatgtc
atcccaggcctccaagagcaaagccactgaggtatctctacaaaacccac
cagagtctggcactgatggttgcattttgttaattgttctaagttggtgg
agcagaactttgcctacttatgtttattgtcaaatgcttctatgcccatt
tccattccctccataa
FANCD2 SPLICE VARIANT 1

(SEQ ID NO: 2)

MVSKRRLSKSEDKESLTEDASKTRKQPLSKKTKKSHIANEVEENDSIFVK
LLKISGIILKTGESQNQLAVDQIAFQKKLFQTLRRHPSYPKIIEEFVSGL
ESYIEDEDSFRNCLLSCERLQDEEASMGASYSKSLIKLLLGIDILQPAII
KTLFEKLPEYFFENKNSDEINIPRLIVSQLKWLDRVVDGKDLTTKIMQLI
SIAPENLQHDIITSLPEILGDSQHADVGKELSDLLIENTSLTVPILDVLS
SLRLDPNFLLKVRQLVMDKLSSIRLEDLPVIIKFILHSVTAMDTLEVISE
LREKLDLQHCVLPSRLQASQVKLKSKGRASSSGNQESSGQSCIILLFDVI
KSAIRYEKTISEAWIKAIENTASVSEHKVFDLVMLFIIYSTNTQTKKYID
RVLRNKIRSGCIQEQLLQSTFSVHYLVLKDMCSSILSLAQSLLHSLDQSI
ISFGSLLYKYAFKFFDTYCQQEVVGALVTHICSGNEAEVDTALDVLLELV
VLNPSAMMMNAVFVKGILDYLDNISPQQIRKLFYVLSTLAFSKQNEASSH
IQDDMHLVIRKQLSSTVFKYKLIGIIGAVTMAGIMAADRSESPSLTQERA
NLSDEQCTQVTSLLQLVHSCSEQSPQASALYYDEFANLIQHEKLDPKALE
WVGHTICNDFQDAFVVDSCVVPEGDFPFPVKALYGLEEYDTQDGIAINLL
PLLFSQDFAKDGGPVTSQESGQKLVSPLCLAPYFRLLRLCVERQHNGNLE
EIDGLLDCPIFLTDLEPGEKLESMSAKERSFMCSLIFLTLNWFREIVNAF
CQETSPEMKGKVLTRLKHIVELQIILEKYLAVTPDYVPPLGNFDVETLDI
TPHTVTAISAKIRKKGKIERKQKTDGSKTSSSDTLSEEKNSECDPTPSHR
GQLNKEFTGKEEKTSLLLHNSHAFFRELDIEVFSILHCGLVTKFILDTEM
HTEATEVVQLGPPELLFLLEDLSQKLESMLTPPIARRVPFLKNKGSRNIG
FSHLQQRSAQEIVHCVFQLLTPMCNHLENIHNYFQCLAAENHGVVDGPGV
KVQEYHIMSSCYQRLLQIFHGLFAWSGFSQPENQNLLYSALHVLSSRLKQ
GEHSQPLEELLSQSVHYLQNFHQSIPSFQCALYLIRLLMVILEKSTASAQ
NKEKIASLARQFLCRVWPSGDKEKSNISNDQLHALLCIYLEHTESILKAI
EEIAGVGVPELINSPKDASSSTFPTLTRHTFVVFFRVMMAELEKTVKKIE
PGTAADSQQIHEEKLLYWNMAVRDFSILINLIKVFDSHPVLHVCLKYGRL
FVEAFLKQCMPLLDFSFRKHREDVLSLLETFQLDTRLLHHLCGHSKIHQD
TRLTQHVPLLKKTLELLVCRVKAMLTLNNCREAFWLGNLKNRDLQGEEIK
SQNSQESTADESEDDMSSQASKSKATEVSLQNPPESGTDGCILLIVLSWW
SRTLPTYVYCQMLLCPFPFPP
FANCD2 SPLICE VARIANT 2 (NM_001018115)

(SEQ ID NO: 3)

atggtttccaaaagaagactgtcaaaatctgaggataaagagagcctgac
agaagatgcctccaaaaccaggaagcaaccactttccaaaaagacaaaga
aatctcatattgctaatgaagttgaagaaaatgacagcatctttgtaaag
cttcttaagatatcaggaattattcttaaaacgggagagagtcagaatca
actagctgtggatcaaatagctttccaaaagaagctctttcagaccctga
ggagacacccttcctatcccaaaataatagaagaatttgttagtggcctg
gagtcttacattgaggatgaagacagtttcaggaactgccttttgtcttg
tgagcgtctgcaggatgaggaagccagtatgggtgcatcttattctaaga
gtctcatcaaactgcttctggggattgacatactgcagcctgccattatc
aaaaccttatttgagaagttgccagaatatttttttgaaaacaagaacag
tgatgaaatcaacatacctcgactcattgtcagtcaactaaaatggcttg
acagagttgtggatggcaaggacctcaccaccaagatcatgcagctgatc
agtattgctccagagaacctgcagcatgacatcatcaccagcctacctga
gatcctaggggattcccagcacgctgatgtggggaaagaactcagtgacc
tactgatagagaatacttcactcactgtcccaatcctggatgtcctttca
agcctccgacttgacccaaacttcctattgaaggttcgccagttggtgat
ggataagttgtcgtctattagattggaggatttacctgtgataataaagt
tcattcttcattccgtaacagccatggatacacttgaggtaatttctgag
cttcgggagaagttggatctgcagcattgtgttttgccatcacggttaca
ggcttcccaagtaaagttgaaaagtaaaggacgagcaagttcctcaggaa
atcaagaaagcagcggtcagagctgtattattctcctctttgatgtaata
aagtcagctattagatatgagaaaaccatttcagaagcctggattaaggc
aattgaaaacactgcctcagtatctgaacacaaggtgtttgacctggtga
tgcttttcatcatctatagcaccaatactcagacaaagaagtacattgac
agggtgctaagaaataagattcgatcaggctgcattcaagaacagctgct
ccagagtacattctctgttcattacttagttcttaaggatatgtgttcat
ccattctgtcgctggctcagagtttgcttcactctctagaccagagtata
atttcatttggcagtctcctatacaaatatgcatttaagttttttgacac
gtactgccagcaggaagtggttggtgccttagtgacccatatctgcagtg
ggaatgaagctgaagttgatactgccttagatgtccttctagagttggta
gtgttaaacccatctgctatgatgatgaatgctgtctttgtaaagggcat
tttagattatctggataacatatcccctcagcaaatacgaaaactcttct
atgttctcagcacactggcatttagcaaacagaatgaagccagcagccac
atccaggatgacatgcacttggtgataagaaagcagctctctagcaccgt
attcaagtacaagctcattgggattattggtgctgtgaccatggctggca
tcatggcggcagacagaagtgaatcacctagtttgacccaagagagagcc
aacctgagcgatgagcagtgcacacaggtgacctccttgttgcagttggt
tcattcctgcagtgagcagtctcctcaggcctctgcactttactatgatg
aatttgccaacctgatccaacatgaaaagctggatccaaaagccctggaa
tgggttgggcataccatctgtaatgatttccaggatgccttcgtagtgga
ctcctgtgttgttccggaaggtgactttccatttcctgtgaaagcactgt
acggactggaagaatacgacactcaggatgggattgccataaacctcctg
ccgctgctgttttctcaggactttgcaaaagatgggggtccggtgacctc
acaggaatcaggccaaaaattggtgtctccgctgtgcctggctccgtatt
tccggttactgagactttgtgtggagagacagcataacggaaacttggag
gagattgatggtctactagattgtcctatattcctaactgacctggagcc
tggagagaagttggagtccatgtctgctaaagagcgttcattcatgtgtt
ctctcatatttcttactctcaactggttccgagagattgtaaatgccttc
tgccaggaaacatcacctgagatgaaggggaaggtgctcactcggttaaa
gcacattgtagaattgcaaataatcctggaaaagtacttggcagtcaccc
cagactatgtccctcctcttggaaactttgatgtggaaactttagatata
acacctcatactgttactgctatttcagcaaaaatcagaaagaaaggaaa
aatagaaaggaaacaaaaaacagatggcagcaagacatcctcctctgaca
cactttcagaagagaaaaattcagaatgtgaccctacgccatctcataga
ggccagctaaacaaggagttcacagggaaggaagaaaagacatcattgtt
actacataattcccatgcttttttccgagagctggacattgaggtcttct
ctattctacattgtggacttgtgacgaagttcatcttagatactgaaatg
cacactgaagctacagaagttgtgcaacttgggccccctgagctgctttt
cttgctggaagatctctcccagaagctggagagtatgctgacacctccta
ttgccaggagagtcccctttctcaagaacaaaggaagccggaatattgga
ttctcacatctccaacagagatctgcccaagaaattgttcattgtgtttt
tcaactgctgaccccaatgtgtaaccacctggagaacattcacaactatt
ttcagtgtttagctgctgagaatcacggtgtagttgatggaccaggagtg
aaagttcaggagtaccacataatgtcttcctgctatcagaggctgctgca
gatttttcatgggctttttgcttggagtggattttctcaacctgaaaatc
agaatttactgtattcagccctccatgtccttagtagccgactgaaacag
ggagaacacagccagcctttggaggaactactcagccagagcgtccatta
cttgcagaatttccatcaaagcattcccagtttccagtgtgctctttatc
tcatcagacttttgatggttattttggagaaatcaacagcttctgctcag
aacaaagaaaaaattgcttcccttgccagacaattcctctgtcgggtgtg
gccaagtggggataaagagaagagcaacatctctaatgaccagctccatg
ctctgctctgtatctacctggagcacacagagagcattctgaaggccata
gaggagattgctggtgttggtgtcccagaactgatcaactctcctaaaga
tgcatcttcctccacattccctacactgaccaggcatacttttgttgttt
tcttccgtgtgatgatggctgaactagagaagacggtgaaaaaaattgag
cctggcacagcagcagactcgcagcagattcatgaagagaaactcctcta
ctggaacatggctgttcgagacttcagtatcctcatcaacttgataaagg
tatttgatagtcatcctgttctgcatgtatgtttgaagtatgggcgtctc
tttgtggaagcatttctgaagcaatgtatgccgctcctagacttcagttt
tagaaaacaccgggaagatgttctgagcttactggaaaccttccagttgg
acacaaggctgcttcatcacctgtgtgggcattccaagattcaccaggac
acgagactcacccaacatgtgcctctgctcaaaaagaccctggaactttt
agtttgcagagtcaaagctatgctcactctcaacaattgtagagaggctt
tctggctgggcaatctaaaaaaccgggacttgcagggtgaagagattaag
tcccaaaattcccaggagagcacagcagatgagagtgaggatgacatgtc
atcccaggcctccaagagcaaagccactgaggatggtgaagaagacgaag
taagtgctggagaaaaggagcaagatagtgatgagagttatgatgactct
gattag
FANCD2 SPLICE VARIANT 2

(SEQ ID NO: 4)

MVSKRRLSKSEDKESLTEDASKTRKQPLSKKTKKSHIANEVEENDSIFVK
LLKISGIILKTGESQNQLAVDQIAFQKKLFQTLRRHPSYPKIIEEFVSGL
ESYIEDEDSFRNCLLSCERLQDEEASMGASYSKSLIKLLLGIDILQPAII
KTLFEKLPEYFFENKNSDEINIPRLIVSQLKWLDRVVDGKDLTTKIMQLI
SIAPENLQHDIITSLPEILGDSQHADVGKELSDLLIENTSLTVPILDVLS
SLRLDPNFLLKVRQLVMDKLSSIRLEDLPVIIKFILHSVTAMDTLEVISE
LREKLDLQHCVLPSRLQASQVKLKSKGRASSSGNQESSGQSCIILLFDVI
KSAIRYEKTISEAWIKAIENTASVSEHKVFDLVMLFIIYSTNTQTKKYID
RVLRNKIRSGCIQEQLLQSTFSVHYLVLKDMCSSILSLAQSLLHSLDQSI
ISFGSLLYKYAFKFFDTYCQQEVVGALVTHICSGNEAEVDTALDVLLELV
VLNPSAMMMNAVFVKGILDYLDNISPQQIRKLFYVLSTLAFSKQNEASSH
IQDDMHLVIRKQLSSTVFKYKLIGIIGAVTMAGIMAADRSESPSLTQERA
NLSDEQCTQVTSLLQLVHSCSEQSPQASALYYDEFANLIQHEKLDPKALE
WVGHTICNDFQDAFVVDSCVVPEGDFPFPVKALYGLEEYDTQDGIAINLL
PLLFSQDFAKDGGPVTSQESGQKLVSPLCLAPYFRLLRLCVERQHNGNLE
EIDGLLDCPIFLTDLEPGEKLESMSAKERSFMCSLIFLTLNWFREIVNAF
CQETSPEMKGKVLTRLKHIVELQIILEKYLAVTPDYVPPLGNFDVETLDI
TPHTVTAISAKIRKKGKIERKQKTDGSKTSSSDTLSEEKNSECDPTPSHR
GQLNKEFTGKEEKTSLLLHNSHAFFRELDIEVFSILHCGLVTKFILDTEM
HTEATEVVQLGPPELLFLLEDLSQKLESMLTPPIARRVPFLKNKGSRNIG
FSHLQQRSAQEIVHCVFQLLTPMCNHLENIHNYFQCLAAENHGVVDGPGV
KVQEYHIMSSCYQRLLQIFHGLFAWSGFSQPENQNLLYSALHVLSSRLKQ
GEHSQPLEELLSQSVHYLQNFHQSIPSFQCALYLIRLLMVILEKSTASAQ
NKEKIASLARQFLCRVWPSGDKEKSNISNDQLHALLCIYLEHTESILKAI
EEIAGVGVPELINSPKDASSSTFPTLTRHTFVVFFRVMMAELEKTVKKIE
PGTAADSQQIHEEKLLYWNMAVRDFSILINLIKVFDSMPVLHVCLKYGRL
FVEAFLKQCMPLLDFSFRKHREDVLSLLETFQLDTRLLHHLCGHSKIHQD
TRLTQHVPLLKKTLELLVCRVKAMLTLNNCREAFWLGNLKNRDLQGEEIK
SQNSQESTADESEDDMSSQASKSKATEDGEEDEVSAGEKEQDSDESYDDS
D

FANCD1 (BRCA2): A member of the FA NNC component identified from the Fanconi anemia complementation group D. An exemplary nucleotide sequence of FANCD1 as found at GENBANK® accession number NM_{—}000059 (as available Apr. 15, 2007) is shown below. Also shown is the amino acid sequence encoded by this nucleotide sequence.

FANCD1 (NM_000059)

(SEQ ID NO: 5)

atgcctattggatccaaagagaggccaacattttttgaaatttttaagac
acgctgcaacaaagcagatttaggaccaataagtcttaattggtttgaag
aactttcttcagaagctccaccctataattctgaacctgcagaagaatct
gaacataaaaacaacaattacgaaccaaacctatttaaaactccacaaag
gaaaccatcttataatcagctggcttcaactccaataatattcaaagagc
aagggctgactctgccgctgtaccaatctcctgtaaaagaattagataaa
ttcaaattagacttaggaaggaatgttcccaatagtagacataaaagtct
tcgcacagtgaaaactaaaatggatcaagcagatgatgtttcctgtccac
ttctaaattcttgtcttagtgaaagtcctgttgttctacaatgtacacat
gtaacaccacaaagagataagtcagtggtatgtgggagtttgtttcatac
accaaagtttgtgaagggtcgtcagacaccaaaacatatttctgaaagtc
taggagctgaggtggatcctgatatgtcttggtcaagttctttagctaca
ccacccacccttagttctactgtgctcatagtcagaaatgaagaagcatc
tgaaactgtatttcctcatgatactactgctaatgtgaaaagctattttt
ccaatcatgatgaaagtctgaagaaaaatgatagatttatcgcttctgtg
acagacagtgaaaacacaaatcaaagagaagctgcaagtcatggatttgg
aaaaacatcagggaattcatttaaagtaaatagctgcaaagaccacattg
gaaagtcaatgccaaatgtcctagaagatgaagtatatgaaacagttgta
gatacctctgaagaagatagtttttcattatgtttttctaaatgtagaac
aaaaaatctacaaaaagtaagaactagcaagactaggaaaaaaattttcc
atgaagcaaacgctgatgaatgtgaaaaatctaaaaaccaagtgaaagaa
aaatactcatttgtatctgaagtggaaccaaatgatactgatccattaga
ttcaaatgtagcacatcagaagccctttgagagtggaagtgacaaaatct
ccaaggaagttgtaccgtctttggcctgtgaatggtctcaactaaccctt
tcaggtctaaatggagcccagatggagaaaatacccctattgcatatttc
ttcatgtgaccaaaatatttcagaaaaagacctattagacacagagaaca
aaagaaagaaagattttcttacttcagagaattctttgccacgtatttct
agcctaccaaaatcagagaagccattaaatgaggaaacagtggtaaataa
gagagatgaagagcagcatcttgaatctcatacagactgcattcttgcag
taaagcaggcaatatctggaacttctccagtggcttcttcatttcagggt
atcaaaaagtctatattcagaataagagaatcacctaaagagactttcaa
tgcaagtttttcaggtcatatgactgatccaaactttaaaaaagaaactg
aagcctctgaaagtggactggaaatacatactgtttgctcacagaaggag
gactccttatgtccaaatttaattgataatggaagctggccagccaccac
cacacagaattctgtagctttgaagaatgcaggtttaatatccactttga
aaaagaaaacaaataagtttatttatgctatacatgatgaaacattttat
aaaggaaaaaaaataccgaaagaccaaaaatcagaactaattaactgttc
agcccagtttgaagcaaatgcttttgaagcaccacttacatttgcaaatg
ctgattcaggtttattgcattcttctgtgaaaagaagctgttcacagaat
gattctgaagaaccaactttgtccttaactagctcttttgggacaattct
gaggaaatgttctagaaatgaaacatgttctaataatacagtaatctctc
aggatcttgattataaagaagcaaaatgtaataaggaaaaactacagtta
tttattaccccagaagctgattctctgtcatgcctgcaggaaggacagtg
tgaaaatgatccaaaaagcaaaaaagtttcagatataaaagaagaggtct
tggctgcagcatgtcacccagtacaacattcaaaagtggaatacagtgat
actgactttcaatcccagaaaagtcttttatatgatcatgaaaatgccag
cactcttattttaactcctacttccaaggatgttctgtcaaacctagtca
tgatttctagaggcaaagaatcatacaaaatgtcagacaagctcaaaggt
aacaattatgaatctgatgttgaattaaccaaaaatattcccatggaaaa
gaatcaagatgtatgtgctttaaatgaaaattataaaaacgttgagctgt
tgccacctgaaaaatacatgagagtagcatcaccttcaagaaaggtacaa
ttcaaccaaaacacaaatctaagagtaatccaaaaaaatcaagaagaaac
tacttcaatttcaaaaataactgtcaatccagactctgaagaacttttct
cagacaatgagaataattttgtcttccaagtagctaatgaaaggaataat
cttgctttaggaaatactaaggaacttcatgaaacagacttgacttgtgt
aaacgaacccattttcaagaactctaccatggttttatatggagacacag
gtgataaacaagcaacccaagtgtcaattaaaaaagatttggtttatgtt
cttgcagaggagaacaaaaatagtgtaaagcagcatataaaaatgactct
aggtcaagatttaaaatcggacatctccttgaatatagataaaataccag
aaaaaaataatgattacatgaacaaatgggcaggactcttaggtccaatt
tcaaatcacagttttggaggtagcttcagaacagcttcaaataaggaaat
caagctctctgaacataacattaagaagagcaaaatgttcttcaaagata
ttgaagaacaatatcctactagtttagcttgtgttgaaattgtaaatacc
ttggcattagataatcaaaagaaactgagcaagcctcagtcaattaatac
tgtatctgcacatttacagagtagtgtagttgtttctgattgtaaaaata
gtcatataacccctcagatgttattttccaagcaggattttaattcaaac
cataatttaacacctagccaaaaggcagaaattacagaactttctactat
attagaagaatcaggaagtcagtttgaatttactcagtttagaaaaccaa
gctacatattgcagaagagtacatttgaagtgcctgaaaaccagatgact
atcttaaagaccacttctgaggaatgcagagatgctgatcttcatgtcat
aatgaatgccccatcgattggtcaggtagacagcagcaagcaatttgaag
gtacagttgaaattaaacggaagtttgctggcctgttgaaaaatgactgt
aacaaaagtgcttctggttatttaacagatgaaaatgaagtggggtttag
gggcttttattctgctcatggcacaaaactgaatgtttctactgaagctc
tgcaaaaagctgtgaaactgtttagtgatattgagaatattagtgaggaa
acttctgcagaggtacatccaataagtttatcttcaagtaaatgtcatga
ttctgttgtttcaatgtttaagatagaaaatcataatgataaaactgtaa
gtgaaaaaaataataaatgccaactgatattacaaaataatattgaaatg
actactggcacttttgttgaagaaattactgaaaattacaagagaaatac
tgaaaatgaagataacaaatatactgctgccagtagaaattctcataact
tagaatttgatggcagtgattcaagtaaaaatgatactgtttgtattcat
aaagatgaaacggacttgctatttactgatcagcacaacatatgtcttaa
attatctggccagtttatgaaggagggaaacactcagattaaagaagatt
tgtcagatttaacttttttggaagttgcgaaagctcaagaagcatgtcat
ggtaatacttcaaataaagaacagttaactgctactaaaacggagcaaaa
tataaaagattttgagacttctgatacattttttcagactgcaagtggga
aaaatattagtgtcgccaaagagtcatttaataaaattgtaaatttcttt
gatcagaaaccagaagaattgcataacttttccttaaattctgaattaca
ttctgacataagaaagaacaaaatggacattctaagttatgaggaaacag
acatagttaaacacaaaatactgaaagaaagtgtcccagttggtactgga
aatcaactagtgaccttccagggacaacccgaacgtgatgaaaagatcaa
agaacctactctgttgggttttcatacagctagcgggaaaaaagttaaaa
ttgcaaaggaatctttggacaaagtgaaaaacctttttgatgaaaaagag
caaggtactagtgaaatcaccagttttagccatcaatgggcaaagaccct
aaagtacagagaggcctgtaaagaccttgaattagcatgtgagaccattg
agatcacagctgccccaaagtgtaaagaaatgcagaattctctcaataat
gataaaaaccttgtttctattgagactgtggtgccacctaagctcttaag
tgataatttatgtagacaaactgaaaatctcaaaacatcaaaaagtatct
ttttgaaagttaaagtacatgaaaatgtagaaaaagaaacagcaaaaagt
cctgcaacttgttacacaaatcagtccccttattcagtcattgaaaattc
agccttagctttttacacaagttgtagtagaaaaacttctgtgagtcaga
cttcattacttgaagcaaaaaaatggcttagagaaggaatatttgatggt
caaccagaaagaataaatactgcagattatgtaggaaattatttgtatga
aaataattcaaacagtactatagctgaaaatgacaaaaatcatctctccg
aaaaacaagatacttatttaagtaacagtagcatgtctaacagctattcc
taccattctgatgaggtatataatgattcaggatatctctcaaaaaataa
acttgattctggtattgagccagtattgaagaatgttgcagatcaaaaaa
acactagtttttccaaagtaatatccaatgtaaaagatgcaaatgcatac
ccacaaactgtaaatgaagatatttgcgttgaggaacttgtgactagctc
ttcaccctgcaaaaataaaaatgcagccattaaattgtccatatctaata
gtaataattttgaggtagggccacctgcatttaggatagccagtggtaaa
atcgtttgtgtttcacatgaaacaattaaaaaagtgaaagacatatttac
agacagtttcagtaaagtaattaaggaaaacaacgagaataaatcaaaaa
tttgccaaacgaaaattatggcaggttgttacgaggcattggatgattca
gaggatattcttcataactctctagataatgatgaatgtagcacgcattc
acataaggtttttgctgacattcagagtgaagaaattttacaacataacc
aaaatatgtctggattggagaaagtttctaaaatatcaccttgtgatgtt
agtttggaaacttcagatatatgtaaatgtagtatagggaagcttcataa
gtcagtctcatctgcaaatacttgtgggatttttagcacagcaagtggaa
aatctgtccaggtatcagatgcttcattacaaaacgcaagacaagtgttt
tctgaaatagaagatagtaccaagcaagtcttttccaaagtattgtttaa
aagtaacgaacattcagaccagctcacaagagaagaaaatactgctatac
gtactccagaacatttaatatcccaaaaaggcttttcatataatgtggta
aattcatctgctttctctggatttagtacagcaagtggaaagcaagtttc
cattttagaaagttccttacacaaagttaagggagtgttagaggaatttg
atttaatcagaactgagcatagtcttcactattcacctacgtctagacaa
aatgtatcaaaaatacttcctcgtgttgataagagaaacccagagcactg
tgtaaactcagaaatggaaaaaacctgcagtaaagaatttaaattatcaa
ataacttaaatgttgaaggtggttcttcagaaaataatcactctattaaa
gtttctccatatctctctcaatttcaacaagacaaacaacagttggtatt
aggaaccaaagtctcacttgttgagaacattcatgttttgggaaaagaac
aggcttcacctaaaaacgtaaaaatggaaattggtaaaactgaaactttt
tctgatgttcctgtgaaaacaaatatagaagtttgttctacttactccaa
agattcagaaaactactttgaaacagaagcagtagaaattgctaaagctt
ttatggaagatgatgaactgacagattctaaactgccaagtcatgccaca
cattctctttttacatgtcccgaaaatgaggaaatggttttgtcaaattc
aagaattggaaaaagaagaggagagccccttatcttagtgggagaaccct
caatcaaaagaaacttattaaatgaatttgacaggataatagaaaatcaa
gaaaaatccttaaaggcttcaaaaagcactccagatggcacaataaaaga
tcgaagattgtttatgcatcatgtttctttagagccgattacctgtgtac
cctttcgcacaactaaggaacgtcaagagatacagaatccaaattttacc
gcacctggtcaagaatttctgtctaaatctcatttgtatgaacatctgac
tttggaaaaatcttcaagcaatttagcagtttcaggacatccattttatc
aagtttctgctacaagaaatgaaaaaatgagacacttgattactacaggc
agaccaaccaaagtctttgttccaccttttaaaactaaatcacattttca
cagagttgaacagtgtgttaggaatattaacttggaggaaaacagacaaa
agcaaaacattgatggacatggctctgatgatagtaaaaataagattaat
gacaatgagattcatcagtttaacaaaaacaactccaatcaagcagcagc
tgtaactttcacaaagtgtgaagaagaacctttagatttaattacaagtc
ttcagaatgccagagatatacaggatatgcgaattaagaagaaacaaagg
caacgcgtctttccacagccaggcagtctgtatcttgcaaaaacatccac
tctgcctcgaatctctctgaaagcagcagtaggaggccaagttccctctg
cgtgttctcataaacagctgtatacgtatggcgtttctaaacattgcata
aaaattaacagcaaaaatgcagagtcttttcagtttcacactgaagatta
ttttggtaaggaaagtttatggactggaaaaggaatacagttggctgatg
gtggatggctcataccctccaatgatggaaaggctggaaaagaagaattt
tatagggctctgtgtgacactccaggtgtggatccaaagcttatttctag
aatttgggtttataatcactatagatggatcatatggaaactggcagcta
tggaatgtgcctttcctaaggaatttgctaatagatgcctaagcccagaa
agggtgcttcttcaactaaaatacagatatgatacggaaattgatagaag
cagaagctcggctataaaaaagataatggaaagggatgacacagctgcaa
aaacacttgttctctgtgtttctgacataatttcattgagcgcaaatata
tctgaaacttctagcaataaaactagtagtgcagatacccaaaaagtggc
cattattgaacttacagatgggtggtatgctgttaaggcccagttagatc
ctcctctcttagctgtcttaaagaatggcagactgacagttggtcagaag
attattcttcatggagcagaactggtgggctctcctgatgcctgtacacc
tcttgaagccccagaatctcttatgttaaagatttctgctaacagtactc
ggcctgctcgctggtataccaaacttggattctttcctgaccctagacct
tttcctctgcccttatcatcgcttttcagtgatggaggaaatgttggttg
tgttgatgtaattattcaaagagcataccctatacagtggatggagaaga
catcatctggattatacatatttcgcaatgaaagagaggaagaaaaggaa
gcagcaaaatatgtggaggcccaacaaaagagactagaagccttattcac
taaaattcaggaggaatttgaagaacatgaagaaaacacaacaaaaccat
atttaccatcacgtgcactaacaagacagcaagttcgtgctttgcaagat
ggtgcagagctttatgaagcagtgaagaatgcagcagacccagcttacct
tgagggttatttcagtgaagagcagttaagagccttgaataatcacaggc
aaatgttgaatgataagaaacaagctcagatccagttggaaattaggaag
gccatggaatctgctgaacaaaaggaacaaggtttatcaagggatgtcac
aaccgtgtggaagttgcgtattgtaagctattcaaaaaaagaaaaagatt
cagttatactgagtatttggcgtccatcatcagatttatattctctgtta
acagaaggaaagagatacagaatttatcatcttgcaacttcaaaatctaa
aagtaaatctgaaagagctaacatacagttagcagcgacaaaaaaaactc
agtatcaacaactaccggtttcagatgaaattttatttcagatttaccag
ccacgggagccccttcacttcagcaaatttttagatccagactttcagcc
atcttgttctgaggtggacctaataggatttgtcgtttctgttgtgaaaa
aaacaggacttgcccctttcgtctatttgtcagacgaatgttacaattta
ctggcaataaagttttggatagaccttaatgaggacattattaagcctca
tatgttaattgctgcaagcaacctccagtggcgaccagaatccaaatcag
gccttcttactttatttgctggagatttttctgtgttttctgctagtcca
aaagagggccactttcaagagacattcaacaaaatgaaaaatactgttga
gaatattgacatactttgcaatgaagcagaaaacaagcttatgcatatac
tgcatgcaaatgatcccaagtggtccaccccaactaaagactgtacttca
gggccgtacactgctcaaatcattcctggtacaggaaacaagcttctgat
gtcttctcctaattgtgagatatattatcaaagtcctttatcactttgta
tggccaaaaggaagtctgtttccacacctgtctcagcccagatgacttca
aagtcttgtaaaggggagaaagagattgatgaccaaaagaactgcaaaaa
gagaagagccttggatttcttgagtagactgcctttacctccacctgtta
gtcccatttgtacatttgtttctccggctgcacagaaggcatttcagcca
ccaaggagttgtggcaccaaatacgaaacacccataaagaaaaaagaact
gaattctcctcagatgactccatttaaaaaattcaatgaaatttctcttt
tggaaagtaattcaatagctgacgaagaacttgcattgataaatacccaa
gctcttttgtctggttcaacaggagaaaaacaatttatatctgtcagtga
atccactaggactgctcccaccagttcagaagattatctcagactgaaac
gacgttgtactacatctctgatcaaagaacaggagagttcccaggccagt
acggaagaatgtgagaaaaataagcaggacacaattacaactaaaaaata
tatctaa
FANCD1

(SEQ ID NO: 6)

MPIGSKERPTFFEIFKTRCNKADLGPISLWFEELSSEAPPYNSEPAEESE
IIKNNNYEPNLFKTPQRKPSYNQLASTPIIFKEQGLTLPLYQSPVKELDK
FKLDLGRNVPNSRHKSLRTVKTKMDQADDVSCPLLNSCLSESPVVLQCTH
VTPQRDKSVVCGSLFHTPKFVKGRQTPKRISESLGAEVDPDMSWSSSLAT
PPTLSSTVLIVRNEEASETVFPHDTTANVKSYFSNHDESLKKNDRFIASV
TDSENTNQREAASHGFGKTSGNSFKVNSCKDHIGKSMPNVLEDEVYETVV
DTSEEDSFSLCFSKCRTKNLQKVRTSKTRKKIFHEANADECEKSKNQVKE
KYSFVSEVEPNDTDPLDSNVAHQKPFESGSDKISKEVVPSLACEWSQLTL
SGLNGAQMEKIPLLHISSCDQNISEKDLLDTENKRKKDFLTSENSLPRIS
SLPKSEKPLNEETVVNKRDEEQHLESHTDCILAVKQAISGTSPVASSFQG
IKKSIFRIRESPKETFNASFSGHMTDPNFKKETEASESGLEIHTVCSQKE
DSLCPNLIDNGSWPATTTQNSVALKWAGLISTLKKKTNKFIYAIHDETFY
KGKKIPKDQKSELINCSAQFEANAFEAPLTFAWADSGLLHSSVKRSCSQN
DSEEPTLSLTSSFGTILRKCSRNETCSNNTVISQDLDYKEAKCNKEKLQL
FITPEADSLSCLQEGQCENDPKSKKVSDIKEEVLAAACHPVQHSKVEYSD
TDFQSQKSLLYDHENASTLILTPTSKDVLSNLVMISRGKESYKMSDKLKG
NNYESDVELTKNIPMEKNQDVCALNENYKNVELLPPERYMRVASPSRKVQ
PNQNTNLRVIQKNQEETTSISKITVNPDSEELFSDNENNFVFQVANERNN
LALGNTKELHETDLTCVNEPIFKNSTMVLYGDTGDKQATQVSIKKDLVYV
LAEENKNSVKQHIKMTLGQDLKSDISLNIDKIPEKNNDYMNKWAGLLGPI
SNHSFGGSFRTASNKEIKLSEHNIKKSKMFFKDIEEQYPTSLACVEIVNT
LALDNQKKLSKPQSINTVSAHLQSSVVVSDCKNSHITPQMLFSKQDFNSN
HNLTPSQKAEITELSTILEESGSQFEFTQFRKPSYILQKSTFEVPENQMT
ILKTTSEECRDADLHVIMNAPSIGQVDSSKQFEGTVEIKRKFAGLLKNDC
NKSASGYLTDENEVGFRGFYSAHGTKLNVSTEALQKAVKLFSDIENISEE
TSAEVHPISLSSSKCHDSVVSMFKIENHNDKTVSEKNNKCQLILQNNIEM
TTGTFVEEITENYKRNTENEDNKYTAASRNSHNLEFDGSDSSKNDTVCIH
KDETDLLFTDQHNICLKLSGQFMKEGNTQIKEDLSDLTFLEVAKAQEACH
GNTSNKEQLTATKTEQNIKDFETSDTFFQTASGKNISVAKESFNKIVNFF
DQKPEELHNFSLNSELHSDIRKNKMDILSYEETDIVKHKILKESVPVGTG
NQLVTFQGQPERDEKIKEPTLLGFHTASGKKVKIAKESLDKVKNLFDEKE
QGTSEITSFSHQWAKTLKYREACKDLELACETIEITAAPKCKEMQNSLNN
DKNLVSIETVVPPKLLSDNLCRQTENLKTSKSIFLKVKVHENVEKETAKS
PATCYTNQSPYSVIENSALAFYTSCSRKTSVSQTSLLEAKKWLREGIFDG
QPERINTADYVGNYLYENNSNSTIAENDKNHLSEKQDTYLSNSSMSNSYS
YHSDEVYNDSGYLSKNKLDSGIEPVLKNVEDQKNTSFSKVISNVKDANAY
PQTVNEDICVEELVTSSSPCKNKNAAIKLSISNSNNFEVGPPAFRIASGK
IVCVSHETIKKVKDIFTDSFSKVIKENNENKSKICQTKIMAGCYEALDDS
EDILHNSLDNDECSTHSHKVFADIQSEEILQHNQNMSGLEKVSKISPCDV
SLETSDICKCSIGKLHKSVSSANTCGIFSTASGKSVQVSDASLQNARQVF
SEIEDSTKQVFSKVLFKSNEHSDQLTREENTAIRTPEHLISQKGFSYNVV
NSSAFSGFSTASGKQVSILESSLHKVKGVLEEFDLIRTEHSLHYSPTSRQ
NVSKILPRVDKRNPEHCVNSEMEKTCSKEFKLSNNLNVEGGSSENNHSIK
VSPYLSQFQQDKQQLVLGTKVSLVENIHVLGKEQASPKNVKMEIGKTETF
SDVPVKTNIEVCSTYSKDSENYFETEAVEIAKAFMEDDELTDSKLPSHAT
HSLFTCPENEEMVLSNSRIGKRRGEPLILVGEPSIKRNLLNEFDRIIENQ
EKSLKASKSTPDGTIKDRRLFMHHVSLEPITCVPFRTTKERQEIQNPNFT
APGQEFLSKSHLYEHLTLEKSSSNLAVSGHPFYQVSATRNEKMRHLITTG
RPTKVFVPPFKTKSHFHRVEQCVRNINLEENRQKQNIDGHGSDDSKNKIN
DNEINQFNKNNSNQAAAVTFTKCEEEPLDLITSLQNARDIQDMRIKKKQR
QRVFPQPGSLYLAKTSTLPRISLKAAVGGQVPSACSHKQLYTYGVSKHCI
KINSKNAESFQFHTEDYFGKESLWTGKGIQLADGGWLIPSNDGKAGKEEF
YRALCDTPGVDPKLISRIWVYNHYRWIIWKLAANECAFPKEFANRCLSPE
RVLLQLKYRYDTEIDRSRRSAIKKIMERDDTAAKTLVLCVSDIISLSANI
SETSSNKTSSADTQKVAIIELTDGWYAVKAQLDPPLLAVLKNGRLTVGQK
IILHGAELVGSPDACTPLEAPESLMLKISANSTRPARWYTKLGFFPDPRP
FPLPLSSLFSDGGNVGCVDVIIQRAYPIQWMEKTSSGLYIFRNEREEEKE
AAKYVEAQQKRLEALFTKIQEEFEEHEENTTKPYLPSRALTRQQVRALQD
GAELYEAVKNAADPAYLEGYFSEEQLRALNNHRQMLNDKKQAQIQLEIRK
AMESAEQKEQGLSRDVTTVWKLRIVSYSKKEKDSVILSIWRPSSDLYSLL
TEGKRYRIYHLATSKSKSKSERANIQLAATKKTQYQQLPVSDEILFQIYQ
PREPLHFSKPLDPDFQPSCSEVDLIGFVVSVVKKTGLAPFVYLSDECYNL
LAIKFWIDLNEDIIKPHMLIAASNLQWRPESKSGLLTLFAGDFSVFSASP
KEGHFQETFNKMKNTVENIDILCNEAENKLMHILHANDPKWSTPTKDCTS
GPYTAQIIPGTGNKLLMSSPNCEIYYQSPLSLCMAKRKSVSTPVSAQMTS
KSCKGEKEIDDQKMCKKRRALDFLSRLPLPPPVSPICTFVSPAAQKAFQP
PRSCGTKYETPIKKKELNSPQMTPFKKFNEISLLESNSIADEELALINTQ
ALLSGSTGEKQFISVSESTRTAPTSSEDYLRLKRRCTTSLIKEQESSQAS
TEECEKNKQDTITTKKYI

FANCJ: A member of the FA NNC component identified from the Fanconi anemia complementation group J, also referred to as BRIP1 or BACH1. The protein encoded by this gene is a member of the RecQ DEAH helicase family. An exemplary nucleotide sequence of FANCJ as found at GENBANK® accession number NM_{—}032043 (as available Mar. 25, 2007) is shown below. Also shown is the amino acid sequence encoded by this nucleotide sequence.

FANCJ (NM_032043)

(SEQ ID NO: 7)

atgtcttcaatgtggtctgaatatacaattggtggggtgaagatttactt
tccttataaagcttacccgtcacagcttgctatgatgaattctattctca
gaggattaaacagcaagcaacattgtttgttggagagtcccacaggaagt
ggaaaaagcttagccttactttgttctgctttagcatggcaacaatctct
tagtgggaaaccagcagatgagggcgtaagtgaaaaagctgaagtacaat
tgtcatgttgttgtgcatgccattcaaaggattttacaaacaatgacatg
aaccaaggaacttcacgtcatttcaactatccaagcacaccaccttctga
aagaaatggcacttcatcaacttgtcaagactcccctgaaaaaaccactc
tggctgcaaagttatctgctaagaaacaggcatccatatacagagatgaa
aatgatgattttcaagtagagaagaaaagaattcgacccttagaaactac
acagcagattagaaaacgtcattgctttggaacagaagtacacaatttgg
atgcaaaagttgattcaggaaagactgtaaaactcaactctccactggaa
aagataaactccttttcgccacagaaaccccctggccactgttctaggtg
ctgttgttctactaaacaaggaaacagtcaagagtcatcgaataccatta
agaaggatcatacagggaaatccaagatacccaaaatatattttgggaca
cgcacacacaagcagattgctcagattactagagagctccggaggacggc
atattcaggggttccaatgactattctttccagcagggatcatacttgtg
tccatcctgaggtagtcggtaacttcaacagaaatgagaagtgcatggaa
ttgctagatgggaaaaacggaaaatcctgctatttttatcatggagttca
taaaattagtgatcagcacacattacagactttccaagggatgtgcaaag
cctgggatatagaagaacttgtcagcctggggaagaaactaaaggcctgt
ccatattacacagcccgagaactaatacaagatgctgacatcatattttg
tccctacaactatcttctagatgcacaaataagggaaagtatggatttaa
atctgaaagaacaggttgtcattttagatgaagctcataacatcgaggac
tgtgctcgggaatcagcaagttacagtgtaacagaagttcagcttcggtt
tgctcgggatgaactagatagtatggtcaacaataatataaggaagaaag
atcatgaacccctacgagctgtgtgctgtagcctcattaattggttagaa
gcaaacgctgaatatcttgtagaaagagattatgaatcagcttgtaaaat
atggagtggaaatgaaatgctcttaactttacacaaaatgggtatcacca
ctgctacttttcccattttgcagggacatttttctgctgttcttcaaaaa
gaggaaaaaatctcaccaatttatggtaaagaggaggcaagagaagtacc
tgttattagtgcatcaactcaaataatgcttaaaggactttttatggtac
ttgactatctttttaggcaaaatagcagatttgcagatgattataaaatt
gcgattcaacagacttactcctggacaaatcagattgatatttcagacaa
aaatgggttgttggttctaccaaaaaataagaaacgttcacgacagaaaa
ctgcagttcatgtgctaaacttttggtgcttaaatccagctgtggccttt
tcagatattaatggcaaagttcagaccattgttttgacatctggtacatt
atcaccaatgaaatccttttcgtcagaacttggtgttacatttactatcc
agctggaggctaatcatatcattaaaaattcacaggtttgggttggtacc
attgggtcaggccccaagggtcggaatctctgtgctaccttccagaatac
tgaaacatttgagttccaagatgaagtgggagcacttttgttatctgtgt
gccagactgtgagccaaggaattttgtgtttcttgccatcttacaagtta
ttagaaaaattaaaagaacgttggctctctactggtttatggcataatct
ggagttggtgaagacagtcattgtagaaccacagggaggagaaaaaacaa
attttgatgaattactgcaggtgtactatgacgcaatcaaatacaaagga
gagaaagatggagctctcctggtagcagtttgtcgtggtaaagtgagtga
gggtctggatttctcagatgacaatgcccgtgctgtcataacaataggaa
ttccttttccaaatgtgaaagatctacaggttgaactaaaacgacaatac
aatgaccaccattcaaaattgagaggtcttctacctggccgtcagtggta
tgaaattcaagcatacagggccttaaaccaggcccttggtagatgtatta
gacacagaaatgattggggagctcttattctagtggatgatcgctttagg
aataacccaagtcgctatatatctggactttctaaatgggtacggcagca
gattcagcaccattcaacctttgaaagtgcactggagtccttggctgaat
tttccaaaaagcatcaaaaagttcttaatgtatccataaaggacagaacc
aatatacaggacaatgagtctacacttgaagtgacctctttaaagtacag
taccccaccttatttactggaagcagcaagtcatctatcaccagaaaatt
ttgtggaagatgaagcaaagatatgtgtccaggaactacagtgtcctaaa
attattaccaaaaattcacctctaccaagtagcattatctccagaaagga
gaaaaatgatccagtattcctggaagaagcagggaaagcagaaaaaattg
tgatttccagatccacaagcccaactttcaacaaacaaacaaagagagtt
agctggtcaagctttaattctttgggacagtattttactggtaaaatacc
gaaggcaacacctgagctcgggtcatcagagaatagtgcctctagtcctc
cccgtttcaaaacagagaagatggaaagtaaaactgttttgcccttcact
gataaatgtgaatcctcaaatctgacagtaaacacatcgtttggatcatg
ccctcaatcagaaaccattatttcatcattaaagattgatgccaccctta
ctagaaaaaatcattctgaacatccgctctgttctgaagaagccctggat
ccagacattgaattgtctctagtaagtgaagaagataaacagtccacttc
aaatagagattttgaaacagaagcagaagatgaatctatctattttacac
ctgaactttacgatcctgaagatacagatgaagaaaaaaatgacctagct
gaaactgatagaggaaatagattggctaacaattcagattgcattttagc
taaagacctttttgaaattagaactataaaagaagtagattcagccagag
aagtgaaagctgaggattgcatagatacaaagttgaatggaattctgcat
attgaagaaagtaaaattgatgacattgatggtaatgtaaaaacaacttg
gataaatgaactggaactgggaaaaactcatgaaatagaaataaagaact
ttaaaccatctccttccaaaaataaaggcatgtttcctggttttaagtaa
FANCJ

(SEQ ID NO: 8)

MSSMWSEYTIGGVKIYFPYKAYPSQLAMMNSILRGLNSKQHCLLESPTGS
GKSLALLCSALAWQQSLSGKPADEGVSEKAEVQLSCCCACHSKDFTNNDM
NQGTSRHFNYPSTPPSERNGTSSTCQDSPEKTTLAAKLSAKKQASIYRDE
NDDFQVEKKRIRPLETTQQIRKRHCFGTEVHNLDAKVDSGKTVKLNSPLE
KINSFSPQKPPGHCSRCCCSTKQGNSQESSNTIKKDHTGKSKIPKIYFGT
RTHKQIAQITRELRRTAYSGVPMTILSSRDHTCVHPEVVGNFNRNEKCME
LLDGKNGKSCYFYHGVHKISDQHTLQTFQGMCKAWDIEELVSLGKKLKAC
PYYTARELIQDADIIFCPYNYLLDAQIRESMDLNLKEQVVILDEAHNIED
CARESASYSVTEVQLRFARDELDSMVNNNIRKKDHEPLRAVCCSLINWLE
ANAEYLVERDYESACKIWSGNEMLLTLHKMGITTATFPILQGHFSAVLQK
EEKISPIYGKEEAREVPVISASTQIMLKGLFMVLDYLFRQNSRFADDYKI
AIQQTYSWTNQIDISDKNGLLVLPKNKKRSRQKTAVHVLNFWCLNPAVAF
SDINGKVQTIVLTSGTLSPMKSFSSELGVTFTIQLEANHIIKNSQVWVGT
IGSGPKGRNLCATFQNTETFEFQDEVGALLLSVCQTVSQGILCFLPSYKL
LEKLKERWLSTGLWHNLELVKTVIVEPQGGEKTNFDELLQVYYDAIKYKG
EKDGALLVAVCRGKVSEGLDFSDDNARAVITIGIPFPNVKDLQVELKRQY
NDHHSKLRGLLPGRQWYEIQAYRALNQALGRCIRHRNDWGALILVDDRFR
NNPSRYISGLSKWVRQQIQHHSTFESALESLAEFSKKHQKVLNVSIKDRT
NIQDNESTLEVTSLKYSTPPYLLEAASHLSPENFVEDEAKICVQELQCPK
IITKNSPLPSSIISRKEKNDPVFLEEAGKAEKIVISRSTSPTFNKQTKRV
SWSSFNSLGQYFTGKIPKATPELGSSENSASSPPRFKTEKMESKTVLPFT
DKCESSNLTVTNTSFGSCPQSETIISSLKIDATLTRKNHSEHPLCSEEAL
DPDIELSLVSEEDKQSTSNRDFETEAEDESIYFTPELYDPEDTDEEKNDL
AETDRGNRLANNSDCILAKDLFEIRTIKEVDSAREVKAEDCIDTKLNGIL
HIEESKIDDIDGNVKTTWTNELELGKTHEIEIKNFKPSPSKNKGMFPGFK

Female reproductive tissue: Tissue expressed in female reproductive organs, for example breast tissue and gynecological tissue, such as uterine tissue, cervical tissue, ovarian tissue, and vaginal tissue.

High throughput technique: Through a combination of modern robotics, data processing and control software, liquid handling devices, and sensitive detectors, high throughput techniques allows the rapid screening of potential pharmaceutical agents in a short period of time. Through this process, one can rapidly identify active compounds, antibodies, or genes, which affect the FA NNC component.

Increased risk: As used herein “increased risk” of cancer refers to an increase in the statistical probability of developing cancer relative to the general population. For example, risk factor such as a family history of breast and/or ovarian cancer can increase the risk of a subject developing breast and/or ovarian cancer. In another example, a reduction in the activity of the FA NNC component can increase the risk of a subject developing breast and/or ovarian cancer.

Inhibiting or treating a disease: Inhibiting the development of a disease or condition, for example, in a subject who is at risk for a disease or has been diagnosed with such as a tumor (for example, a breast cancer tumor or an ovarian cancer tumor). “Treatment” includes a therapeutic intervention that ameliorates a sign or symptom of a disease or pathological condition after it has begun to develop. A “treatment” also may be used to reduce risk or incidence of metastasis. The beneficial effects or treatment can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other parameters well known in the art that are specific to the particular disease. A “prophylactic” treatment is a treatment for the purpose of decreasing the risk of developing pathology and is typically administered to a subject who does not exhibit signs of a disease or exhibits only early signs of the disease.

Isolated: An “isolated” biological component (such as a nucleic acid, protein, cell (or plurality of cells), tissue, or organelle) has been substantially separated or purified away from other biological components of the organism in which the component naturally occurs for example other tissues, cells, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids

Label: An agent capable of detection, for example by ELISA, spectrophotometry, flow cytometry, or microscopy. For example, a label can be attached to a nucleic acid molecule such as the probes disclosed herein or protein, such as an antibody, thereby permitting detection of the nucleic acid molecule or protein (for example for the detection of a gene product from one or more members of the FA NNC component, such as FANCD1, FANCD2, and/or FANCJ. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Mass spectrometry: Mass spectrometry (mass spec) is a method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single- and double-stranded forms of DNA.

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single- and double-stranded forms of DNA. In one example, a FANCD2 polynucleotide is a nucleic acid encoding a FANCD2 polypeptide.

Polypeptide: Any chain of amino acids, regardless of length or post-translational modification (such as glycosylation, methylation, ubiquitination, phosphorylation, or the like). “Post-translational modification” is the chemical modification of a polypeptide after its translation, for example by monoubiquitination, glycosylation, methylation, phosphorylation, or the like. In one example, FANCD2 is post-translationally modified by ubiquitination, and/or phosphorylation. Post-translational modification can lead to an apparent difference in molecular weight, for example, a difference in molecular weight between post-translationally modified protein, such as a Fanconi anemia protein and the same Fanconi anemia protein, which is not post-translationally modified. This difference can be measured on the basis of a post-translationally modification dependent protein mobility shift, for example on a SDS-PAGE gel or by other methods such as mass spec. In one example, the protein is FANCD2. Thus, post-translationally modified FANCD2 and non-post-translationally modified FANCD2 can be separated by apparent molecular weight.

“Ubiquitin” is a small protein that is ubiquitous in eukaryotes. “Ubiquitination” (or “Ubiquitylation”) refers to the post-translational modification of a protein by the covalent attachment (via an isopeptide bond) of one or more ubiquitin monomers. Monoubiquitination is the process in which a single ubiquitin peptide is bound to a substrate. Poly-ubiquitination is the process in which a chain of ubiquitin peptides are attached to a lysine on a substrate protein. Poly-ubiquitination most commonly results in the degradation of the substrate protein via the proteasome.

“Phosphorylation” is the addition of a phosphate to a protein, typically by a kinase. Measurable phosphorylation of a polypeptide, such as a protein can be quantified using well known assays. This can be done by measuring the incorporation of a radioactive isotope of phosphorous into a test protein, for example the incorporation of [³²P] from the γ phosphate of [γ-³²P]ATP, into a Fanconi anemia protein, such as FANCD2. Phosphorylation also can be measured on the basis of a phosphorylation-dependent protein mobility shift, for example a phosphorylation dependent mobility shift of phosphorylated FANCD2.

Probes and primers: A probe comprises an isolated nucleic acid usually attached to a detectable label or reporter molecule. Primers are short nucleic acids, and can be DNA oligonucleotides 15 nucleotides or more in length. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example, by the polymerase chain reaction (PCR) or other nucleic-acid amplification methods known in the art. One of skill in the art will appreciate that the specificity of a particular probe or primer typically increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation.

Risk factor: A factor that can increase the statistical likelihood of developing a disease, such as cancer. Examples of risk factors for cancer include age and a family history of certain cancers, such as breast or ovarian cancer.

Subject: Living multi-cellular vertebrate organisms, a category that includes both human and veterinary subjects, including human and non-human mammals.

Tissue: A plurality of functionally related cells. A tissue can be a suspension, a semi-solid, or solid. Tissue includes cells collected from a subject such as blood, cervix, uterus, lymph nodes breast, skin, and other organs.

### III. Description of Several Embodiments

The American Cancer Society has estimated that the number of women newly diagnosed with an existing breast and/or ovarian cancer in 2006 will reach approximately 233,000 in the United States, and deaths from these cancers will exceed 55,000. Many of these deaths could be prevented with early detection and proper intervention. The presence of a mutation in either the BRCA1 or BRCA2 gene is an established marker for the predisposition for developing breast or ovarian cancer. However, because of the relatively low incidence of BRCA1 and BRCA2 mutations in breast or ovarian cancer, screening of BRCA1 and BRCA2 cannot accurately identify all subjects with breast and or ovarian cancer or a predisposition to developing ovarian and breast cancer.

Ovarian carcinomas arise from the ovarian surface epithelium (OSE), a continuous single layer of mesothelial cells covering the ovary. Neoplastic ovarian epithelial cells often show signs of genetic instability, both numerical and structural. As disclosed herein, such neoplastic ovarian epithelial cells show hypersensitivity to DNA cross-linking agents, such as MMC and DEB, a response typical of Fanconi anemia (FA). The present disclosure identifies a previously un-described correspondence between tissue specific disruption of expression of particular members of the FA pathway in ovarian and breast cancer. As disclosed herein, the decreased ability of cells to repair DNA damage is correlated with decreases in the expression of FA NNC component gene products. Women with decreased expression of one or more of FANCD2, FANCD1, and FANCJ (designated collectively herein the FA NNC component) are predisposed to developing breast and/or ovarian cancer. The FA NNC component gene products include without limitation the gene products of FANCD2, FANCJ, and FANCD1. It will be appreciated by one of ordinary skill in the art that the gene products of FANCD2, FANCJ, and FANCD1 can be nucleic acids, proteins, or both.

Based on this discovery, the present disclosure provides methods for diagnosing subjects as having breast and/or ovarian cancer or predisposed to developing such cancers. The disclosed methods involve detecting a decrease in activity of the FA NNC component. Detection of a decrease in activity of the FA NNC component can be determined by detecting a decrease in expression of one or more members of the FA NNC component, for example by detecting a decrease in expression of FANCD1, FANCD2, and/or FANCJ. Furthermore, the disclosed methods include detecting a decrease in expression of any or all of FANCD2, FANCD1, and FANCJ, including any one of them alone or any combination of the three, such as

FANCD1 alone;
FANCD2 alone;
FANCJ alone;
FANCD1 and FANCD2;
FANCD1 and FANCJ;
FANCD2 and FANCJ; or
FANCD1, FANCD2, and FANCJ.

Alternatively, the decrease in the activity of the FA NNC component can be determined by biological function, for example by detecting an increase in one or more of chromosomal breakages and radial formations in response to a DNA damaging agent.

Aspect of the disclosed methods are directed to identifying decreases in the activity of the FA NNC component in female reproductive tissue, such as ovarian tissue and/or breast tissue. As disclosed herein, decreases in the activity of the FA NNC component can be characterized by a decrease in the ability of cells to repair induced DNA damage. Accordingly, aspects of the disclosed methods provide for monitoring the ability of cells to repair DNA damage after treatment with a DNA damaging agent. In some embodiments, these methods are used to determine if a subject has ovarian and/or breast cancer or a predisposition for the development of breast and/or ovarian cancer. In some embodiments, the disclosed methods are used to monitor the progression of ovarian and/or breast cancer, for example monitoring the response to a treatment for ovarian and/or breast cancer. In other embodiments, the disclosed methods are used to screen and/or select compounds useful in treating breast and/or ovarian cancer.

### Methods of Diagnosis

The high death rate of subjects with ovarian and/or breast cancer could be improved if methods were available to identify such cancers in subjects prior to or early in their development. Methods for diagnosing these cancers, for example by identifying early malignant tissues or even identifying a predisposition to developing these cancers prior to the occurrence of malignant cell changes involved in the metastasis of these tumor types, is especially important in high risk subjects, such as those with a family history of breast and/or ovarian cancer. This disclosure provides for diagnosing ovarian and/or breast cancer including the predisposition for developing breast and/or ovarian cancer, for example prior to the onset of symptoms, and/or prior to the occurrence of morphological and physiological changes associated with malignancy. Although these methods are applicable to the general population, these methods are particularly useful for diagnosing those individuals with significant risk factors for developing disease.

The activity of the FA NNC component in ovarian epithelial cells from normal, high-risk women, and from women with ovarian cancer can be determined by evaluating chromosome damage in response to DNA alkylating agents (see Table 1). Histologically normal ovarian epithelial cells from a high proportion of women with a predisposition to breast and/or ovarian cancer exhibit increased chromosome breakage in response to the DNA alkylating agents MMC and DEB (Table 1, fourth column). However, lymphocytes from the same subjects taken at the same time point reveal no such chromosomal instability (Table 1, fifth column). This genetic instability is regardless of whether the subject has a genomic mutation in BRCA1 or BRCA2. Thus, whereas genomic mutations in BRCA1 and BRCA2 are infrequent even in women with highly suggestive family histories; reduced activity of the FA NNC component is both frequent and predictive of ovarian and breast cancer.

### Chromosome Breakage and Radial Formation

Aspect of the disclosed methods concern detecting decreases in the activity of the FA NNC component associated with breast and/or ovarian cancer and a predisposition to developing breast and/or ovarian cancer. In some embodiments, this involves detecting the biological function of the FA NNC component, for example by determining the response of cells of female reproductive tissue to DNA damaging agents, for example a DNA damaging agent such as a chemical crosslinking agents for example mitomycin C (MMC), diepoxybutane (DEB), cis diamminedichloroplatinum (cisplatin), cyclophosphamide, psoralen, or radiation such as UVA irradiation.

In some embodiments, the decrease in activity of the FA NNC component is determined by detecting an increase in chromosomal breakage and/or radial formation in response to a DNA damaging agent. In some examples, at least one cell (for example one or more isolated cells, such as cells of female reproductive tissue from a subject) is provided. The cells of the female reproductive tissue are contacted with at least one DNA damaging agent (for example a DNA crosslinking agent) and chromosomal breakage and radial formation is detected in the cell(s). An increase in one or more of chromosomal breakage and radial formation (for example relative to a control) indicates a subject has ovarian and/or breast cancer or is predisposed to developing ovarian and/or breast cancer. Examples, of suitable crosslinking agents for use in the disclosed methods include alkylating agents, for example mitomycin C (MMC) and diepoxybutane (DEB), although any agent that produces crosslinks in sufficient quantity can be used.

In some embodiments, detecting an increase in chromosomal breakage and/or radial formation is made in comparison to a control. Examples of controls of use in the disclosed methods include immortalized ovarian epithelial cells, ovarian cells obtained from subjects that do not have ovarian cancer, cells from subjects that do not have any known risk factors for ovarian and/or breast cancer, cells from ovarian tissue from the subject at an earlier time point (for example, prior to onset of ovarian cancer) non-reproductive tissue obtained from the subject, for example blood cells, such as leukocytes, for example lymphocytes, or statistical controls. In some examples, the control is a standard level of chromosomal breakage established from such cells.

By way of example, cells obtained from the breast and/or reproductive tissue of a subject are exposed to a DNA alkylating agent, such as MMC. The cells are visually assessed for radial formation and/or chromosomal breakage. Increases in the number of radial formations and/or chromosomal breakages, relative to a control, for example a threshold value indicative of a normal tissue, indicate a decrease in the activity of the FA NNC component, and ovarian and or breast cancer in the subject or a predisposition for developing breast and/or ovarian cancer. In a typical MMC assay, cells are exposed in vitro to 0, 20, and 40 micro-molar MMC for three days. Cells are then exposed to colcemid to trigger metaphase arrest. Cells in metaphase are placed on microscopic slides, stained with Wright's stain, and examined microscopically for chromosomal breaks and radial forms. One of ordinary skill in the art will appreciate that any method for determining the number of chromosomal breakages and/or radial formations can be employed.

### Expression of FA Pathway Gene Products

Certain embodiments of the methods disclosed herein involve determining whether there is a decrease in expression of one or more of a FANCJ, FANCD2, and FANCD1 gene product in a sample (such as a tissue sample, for example an ovarian tissue sample or a breast tissue sample) obtained from a subject, such as a human subject. Decreased expression of a FANCJ, FANCD2, or FANCD1 gene product indicates ovarian and/or breast cancer or a predisposition to developing ovarian and/or breast cancer.

In some embodiments, detecting the decrease in activity of the FA NNC component is performed by detecting a decrease in expression of one or more of a FANCD2, FANCD1, and FANCJ gene product in a cell of a subject, such as a cell obtained from a subject's tissue, relative to a control (for example a immortalized ovarian epithelial cells, ovarian cells obtained from subjects that do not have ovarian cancer, cells from subjects that do not have any known risk factors for ovarian and/or breast cancer, cells from ovarian tissue from the subject at an earlier time point (for example, prior to onset of ovarian cancer) non-reproductive tissue obtained from the subject, for example blood cells, such as leukocytes, for example lymphocytes, or statistical controls). A decrease in expression of one or more of the gene products indicates a subject has ovarian and/or breast cancer or a predisposition for the development of ovarian and/or breast cancer. It is understood that a gene product can be either a nucleic acid or a protein. For example, a FANCD2 gene product can be a FANCD2 nucleic acid or a FANCD2 protein, a FANCD1 gene product can be a FANCD1 nucleic acid or a FANCD1 protein, and a FANCJ gene product can be a FANCJ nucleic acid or a FANCJ protein.

In certain embodiments, the detection of a decrease in the activity of the FA NNC component involves detecting a decrease in expression of a FA NNC component nucleic acid associated with breast and/or ovarian cancer. Typically these methods include detecting the expression of at least one nucleic acid, such as a FANCD2 nucleic acid according to SEQ ID NO: 1 or SEQ ID NO: 3, a FANCD1 nucleic acid according to SEQ ID NO: 5, or a FANCJ nucleic acid according to SEQ ID NO: 7.

The alteration of expression of these nucleic acids can be determined simultaneously, for example, the altered expression of FANCD2 and FANCJ, FANCD2 and FANCD1, FANCJ and FANCD1, or FANCJ, FANCD2, and FANCD1 can be determined simultaneously. The expression of these nucleic acids can involve the detection of altered levels of expression of RNA such as mRNA, DNA, such as cDNA, other polynucleotide molecules comprising FANCD1, FANCJ, and FANCD2, or a fragment thereof. In certain embodiments, decreases in expression are detected in more than one molecule, for instance in at least 2 or at least 3 of FANCJ, FANCD2, and FANCD1 nucleic acid molecules.

In some embodiments, decreased expression of FANCJ, FANCD2, and FANCD1 nucleic acid molecules are determined using in vitro nucleic acid amplification and/or nucleic acid hybridization. The results of such detection methods can be quantified, for instance by determining the amount of hybridization or the amount of amplification.

In some embodiments, detecting a decrease in expression of the FANCD2, FANCD1, or FANCJ nucleic acid involves providing a sample of nucleic acids from at least one cell of a subject and detecting the decrease in expression of the FANCD2, FANCD1, or FANCJ nucleic acid in a nucleic acid hybridization assay. A typical hybridization assay proceeds by contacting a sample of nucleic acids from cells of a subject with a target nucleic acid that hybridizes to a FANCD2, FANCD1, or FANCJ nucleic acid. Nucleic acids that can hybridize with a FANCD2, FANCD1, or FANCJ nucleic acid include subsequences of FANCD2, FANCD1, or FANCJ, polynucleotide sequences with at least 95% sequence identity to FANCD2, FANCD1, FANCJ, or subsequences thereof or polynucleotide sequences that hybridize FANCD2, FANCD1, or FANCJ under high stringency conditions. Examples of hybridization assays include Southern blots, Northern blots, and microarrays. FANCD2, FANCD1, or FANCJ nucleic acids can include RNA, DNA, and combinations thereof.

In some embodiments, detecting a decrease in expression of the FANCD2, FANCD1, or FANCJ nucleic acid involves detecting a decrease in expression of the FANCD2, FANCD1, or FANCJ nucleic acid by amplifying at least a portion of the FANCD2, FANCD1, or FANCJ nucleic acid in a quantitative or semi-quantitative amplification assay, for example in an RT-PCR assay. Typical amplification assays are performed with at least one primer that can hybridize with and specifically prime a FANCD2, FANCD1, or FANCJ nucleic acid.

In some embodiments, detecting a decrease in expression of the FANCD2, FANCD1, or FANCJ gene product involves determining the expression of a FANCD2, FANCD1, or FANCJ protein and assessing whether it is reduced, for example compared to a control. Examples of assays for determining the expression of a FANCD2, FANCD1, or FANCJ protein include immunohistochemical assays, radioimmunoassays, Western blot assays, immunofluorescent assays, enzyme immunoassasys, and chemiluminescent assays. Expression of a FANCD2, FANCD1, or FANCJ protein can also be determined by mass-spec analysis.

### Hybridization Assays

In some embodiments of the disclosed methods, detecting a decrease in expression of a FANCD2, FANCD1, or FANCJ nucleic acid involves detecting the hybridization of a target nucleic acid with nucleic acids obtained from a subject, such as nucleic acid obtained from a cell of a subject. Examples of hybridization assays include Southern blots, Northern blots, and microarrays and can include the detection of RNA, DNA, amplification products of nucleic acids, and combinations thereof. Typically, a target nucleic acid is contacted with nucleic acids obtained from a subject, or amplification products of such nucleic acids. The amount of hybridization between the target nucleic acid and nucleic acids obtained from a subject is determined. In certain examples, target nucleic acid sequences are selected such that they specifically hybridize to one or more of FANCD1, FANCD2, and FANCJ nucleic acids. Thus, the sequence of such target nucleic acids can be selected to hybridize specifically to a FANCD2 nucleic acid according to SEQ ID NOs: 1 or 3, a FANCD1 nucleic acid according to SEQ ID NO: 5, or a FANCJ nucleic acid according to SEQ ID NO: 7, for example to hybridize under conditions of high stringency. In specific non-limiting examples, target nucleic acids are selected that hybridize to FANCD2, FANCD1, or FANCJ nucleic acids under high stringency conditions. It will be appreciated that the degree of hybridization stringency required will be dependent the type of hybridization assay used. Methods are provided herein for the selection of hybridization stringency.

Hybridization under moderately or highly stringent conditions excludes non-related nucleotide sequences. In nucleic acid hybridization reactions, the conditions used to achieve a particular level of stringency will vary, depending on the nature of the nucleic acids being hybridized. For example, the length, degree of complementarity, nucleotide sequence composition (such as GC versus AT content), and nucleic acid type (such as RNA versus DNA) of the hybridizing regions of the nucleic acids can be considered in selecting hybridization conditions. An additional consideration is whether one of the nucleic acids is immobilized, for example, on a filter or an array substrate.

Specific hybridization can occur under conditions of varying stringency. Hybridization conditions resulting in particular degrees of stringency will vary depending upon the nature of the hybridization method of choice and the composition and length of the hybridizing DNA used. Generally, the temperature of hybridization and the ionic strength (in particular the Na⁺ concentration) of the hybridization buffer will determine the stringency of hybridization. Calculations regarding hybridization conditions required for attaining particular degrees of stringency are discussed by Sambrook et al. (In: Molecular Cloning: A Laboratory Manual, Cold Spring Harbor, N.Y., 1989 ch. 9 and 11). By way of illustration only, hybridization can be performed by hybridization of a DNA molecule to a target DNA molecule which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989).

A specific, non-limiting example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (see Molecular Cloning: A Laboratory Manual, 2nd ed., Vol. 1-3, ed. Sambrook et al., Cold Spring Harbor Laboratory Press, Cold Spring Harbor, N.Y., 1989). Washing can be carried out using only one of these conditions, for example, high stringency conditions, or each of the conditions can be used, for example, for 10-15 minutes each, in the order listed above, repeating any or all of the steps listed. However, as mentioned above, optimal conditions will vary, depending on the particular hybridization reaction involved, and can be determined empirically.

Traditional hybridization with a target nucleic acid molecule labeled with [³²P]-dCTP is generally carried out in a solution of high ionic strength such as 6×SSC at a temperature that is 20-25° C. below the melting temperature, Tₘ, described below. For Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal.

The term Tₘ represents the temperature (under defined ionic strength, pH and nucleic acid concentration) at which 50% of the probes complementary to the target sequence hybridize to the target sequence at equilibrium. Because the target sequences are generally present in excess, at Tₘ 50% of the probes are occupied at equilibrium. The Tₘ of such a hybrid molecule can be estimated from the following equation (Bolton and McCarthy, Proc. Natl. Acad. Sci. U.S.A. 48:1390, 1962):

Tₘ=81.5° C.−16.6(log₁₀[Na⁺])+0.41(% G+C)−0.63(% forrnamide)−(600/1)

where 1=the length of the hybrid in base pairs.

This equation is valid for concentrations of Na⁺ in the range of 0.01 M to 0.4 M, and it is less accurate for calculations of Tm in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al. (Molecular Cloning: A Laboratory Manual, Cold Spring Harbor, N.Y., 1989).

Thus, by way of example, for a 150 base pair DNA probe derived from a nucleic acid that encodes FANCD2 (e.g., with a hypothetical % GC of 45%), a calculation of hybridization conditions required to give particular stringencies can be made as follows: For this example, it is assumed that the filter will be washed in 0.3×SSC solution following hybridization, thereby: [Na⁺]=0.045 M; % GC=45%; Formamide concentration=0; l=150 base pairs; Tₘ=81.5−16.6(log₁₀[Na+])+(0.41×45)−(600/150); and so Tₘ=74.4° C.

The Tₘ of double-stranded DNA decreases by 1-1.5° C. with every 1% decrease in homology (Bonner et al., J. Mol. Biol. 81:123, 1973). Therefore, for this given example, washing the filter in 0.3×SSC at 59.4-64.4° C. will produce a stringency of hybridization equivalent to 90%; that is, DNA molecules with more than 10% sequence variation relative to the target cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target cDNA molecule will not hybridize. The above example is given entirely by way of theoretical illustration. It will be appreciated that other hybridization techniques can be utilized and that variations in experimental conditions will necessitate alternative calculations for stringency.

Stringent conditions can be defined as those under which DNA molecules with more than 25%, 15%, 10%, 6% 5% 4% 3% 2% or 1% sequence variation (also termed “mismatch”) will not hybridize. Stringent conditions are sequence dependent and are different in different circumstances. Longer sequences hybridize specifically at higher temperatures. Generally, stringent conditions are selected to be about 5° C. lower than the thermal melting point Tₘ for the specific sequence at a defined ionic strength and pH. An example of stringent conditions is a salt concentration of at least about 0.01 to 1.0 M Na ion concentration (or other salts) at pH 7.0 to 8.3 and a temperature of at least about 30° C. for short probes (for example 10 to 50 nucleotides). Stringent conditions can also be achieved with the addition of destabilizing agents such as formamide. For example, conditions of 5×SSPE (750 mM NaCl, 50 mM Na Phosphate, 5 mM EDTA, pH 7.4) and a temperature of 25-30° C. are suitable for allele-specific probe hybridizations.

An alternative method for selecting target nucleic acids is to select nucleic acids that are highly homologous to the nucleic acid sequences of FANCD2, FANCD1, FANCJ, or a subsequence thereof. In specific examples the target nucleic acids are selected such that they are at least about 90% identical to FANCD2 nucleic acid molecule, such as at least about 95%, at least about 98% or at least about 99% identical to a FANCD2 nucleotide sequence according to SEQ ID NO: 1, SEQ ID NO: 3, or a subsequence thereof. In other specific examples the target nucleic acids are selected such that they are at least about 90% identical to FANCD1 nucleic acid molecule, such as at least about 95%, at least about 98% or at least about 99% identical to a nucleotide sequence according to SEQ ID NO: 5 or a subsequence thereof. In other specific examples, the target nucleic acids are selected such that they are at least about 90% identical to FANCJ nucleic acid molecule, such as at least about 95%, at least about 98% or at least about 99% identical to a FANCJ nucleotide according to SEQ ID NO: 7 or a subsequence thereof.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Natl. Acad. Sci. U.S.A. 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. U.S.A. 89:10915, 1989).

A perfectly matched probe has a sequence perfectly complementary to a particular target sequence. The test probe is typically perfectly complementary to a portion (subsequence) of the target sequence. The term “mismatch probe” refers to probes whose sequence is selected not to be perfectly complementary to a particular target sequence.

It will also be recognized that the nucleic acids and the proteins of this disclosure can have variations based on genetic polymorphisms present in the general population such as a single nucleotide polymorphism (SNP). One of ordinary skill in the art will appreciate that a SNP database can be found at http://www.ncbi.nlm.nih.gov/SNP/index.html. For example, FANCD2 splice variant 1 has known single nucleotide polymorphisms at nucleotide positions 1200, 1518, 1587, 2219, 2337, 4176 and 4531 of SEQ ID NO: 1.

### Array Based Assays

In certain embodiments, decreased expression of FANCJ, FANCD2, and FANCD1 nucleic acid molecules are detected using arrays containing two or more nucleic acid molecules. The array may be regular (arranged in uniform rows and columns, for instance) or irregular. Certain embodiments of such arrays are nucleic acid arrays comprising at least one nucleic acid molecule, such as two to more than 5, 10, 20, 25, 30, 45, 50, 55, 60, 65, 75, 100, 150, 200, 250, 500, 550, 600, 650, 700, 750, 800, 850, 900, 950, or 1000. A non-limiting example, is a cDNA microarray which is an array of multiple cDNA molecules in fixed addressable locations, to which complementary nucleic acids can hybridize (see Hegde et al., Biotechniques 29(3): 548-562, 2000). cDNA microarrays of this disclosure provide for qualitative and quantitative analysis of gene expression of the molecules contained in the array.

Within an array, each arrayed sample (feature) is addressable, such that the location of the sample can be reliably and consistently determined. Typically, the location of each sample is assigned to the sample at the time when it is applied to the array. A key can be provided to correlate the location or position of the sample. Arrays are often arranged in a symmetrical grid pattern, although samples could be arranged in any other pattern (for example, radially distributed lines, spiral lines, or ordered clusters). Arrays usually are computer readable, such that a computer can be programmed to correlate a particular address on the array with information about the sample at that position (for example, expression data, which can include signal intensity as well as the identity of the sample).

This disclosure encompasses arrays containing nucleic acid molecules selected to hybridize to FANCD2, FANCJ, or FANCD1 (such as genes, cDNAs or other polynucleotide molecules comprising one or more of FANCD2, FANCJ, or FANCD1, or a fragment thereof). Such arrays can also contain any particular subset of the nucleic acids that hybridize to (or corresponding molecules) of FANCJ, FANCD2, and FANCD1 nucleic acids. Certain arrays (as well as embodiments described herein) also can include nucleic acid molecules that do not hybridize to FANCJ, FANCD2, and FANCD1 nucleic acids. Thus, in one non-limiting example, a nucleic acid array may include nucleic acid molecules selected to hybridize to a FANCJ nucleic acid and a number of nucleic acids that do not hybridize to FANCJ. In another non-limiting example, a nucleic acid array may comprise nucleic acid molecules selected to hybridize to a FANCD2 nucleic acid and a number of nucleic acids that do not hybridize to FANCD2. In another non-limiting example, a nucleic acid array may comprise nucleic acid molecules selected to hybridize to a FANCD1 nucleic acid and a number of nucleic acids that do not hybridize to FANCD1.

Typically, the probes used for nucleic acid arrays comprise an isolated nucleic acid attached to a detectable label or other reporter molecule. These labels may include radioactive isotopes, enzyme substrates, co-factors, ligands, chemiluminescent agents, fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998). Probes typical are used that hybridize to some portion of the target nucleic acid that is present in the array. Hybridization can be made to occur under varying degrees of stringency.

Specific embodiments of the methods for determining a predisposition for breast or ovarian cancer include detecting a decrease in expression of at least one or more or a FANCJ, FANCD2, or FANCD1 molecule use the arrays disclosed herein. Such arrays can be nucleotide (for example, polynucleotide or cDNA) or protein (for example, peptide, polypeptide, or antibody) arrays. In such methods, an array can be contacted with polynucleotides or polypeptides (respectively) from (or derived from) a sample from a subject. The amount and/or position of expression of the subject's polynucleotides or polypeptides then can be determined, for instance to produce a gene expression profile for that subject. Such gene expression profile can be compared to another gene expression profile, for instance a control gene expression profile from a subject having a known ovarian and/or breast cancer-related condition. Optionally, the subject's gene expression profile (sometimes referred to as an expression fingerprint) can be correlated with one or more appropriate treatments, for instance in order to guide treatment choices. Similarly, protein arrays can give rise to protein expression profiles. Both protein and gene expression profiles can more generally be referred to as expression profiles.

### Amplification Assays

Other embodiments of the methods disclosed herein involve amplifying nucleic acids provided from a subject using primers. The sequence of such primers can be selected to specifically hybridize to a FANCD2 nucleic acid molecule, a FANCJ nucleic acid molecule, or a FANCD1 nucleic acid molecule. The amplified nucleic acids can be quantified be any available technique. In specific non-limiting examples, the primers hybridize to FANCD2 nucleic acid molecule, a FANCJ nucleic acid molecule, or a FANCD1 nucleic acid molecule under high stringency conditions. Methods outlined above for the selection of target nucleic acids are equally suitable for the selection of specific primers.

Amplification of a nucleic acid molecule (such as, a DNA or RNA molecule) refers to use of a technique that increases the number of copies of a nucleic acid molecule in a specimen. An example of amplification is the polymerase chain reaction (PCR), in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. This can be repeated as many times as desired. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

In certain embodiments, in vitro amplification can be followed by hybridization. In certain embodiments, the in vitro nucleic acid amplification and/or nucleic acid hybridization are PCR, RT-PCR, real time RT-PCR, quantitative RT-PCR or real time quantitative RT-PCR. In certain embodiments, the probes used will be gene-specific TAQMAN® probes.

### Protein Assays

In other embodiments, the detection of a reduction in activity of the FA NNC component involves detecting altered expression of a FA NNC component protein associated with ovarian and/or breast cancer, such as a FANCD2 protein according to SEQ ID NO: 2 or SEQ ID NO: 4, a FANCJ protein according to SEQ ID NO: 8, or a FANCD1 protein according to SEQ ID NO: 6. It is understood that a fragment of or a portion of FANCD2, FANCJ, or FANCD1 can also be detected. Fragments can include but are not limited to products of enzymatic digestion. Detection of abnormal FANCD1, FANCD2, or FANCJ proteins, which are expressed instead of normal functional proteins is another approach to detecting altered expression of a protein member of the FA NNC component.

It is also encompassed by this disclosure that the aforementioned polypeptides FANCD1, FANCD2, and FANCJ can contain conservative amino acid substitutions. “Conservative” amino acid substitutions are those substitutions that do not substantially affect or decrease an activity or antigenicity of FANCD2, FANCD1, or FANCJ. For example, a FANCD2 polypeptide can include at most about 1, at most about 2, at most about 5, and at most about 10, or at most about 15 conservative substitutions and specifically bind an antibody that binds the original FANCD2 polypeptide. Conservative variations also includes the use of a substituted amino acid in place of an unsubstituted parent amino acid, provided that antibodies raised antibodies raised to the substituted polypeptide also immunoreact with the unsubstituted polypeptide. Non-conservative substitutions are those that reduce an activity or antigenicity.

In some embodiments, decreased expression of FANCJ, FANCD2, and FANCD1 proteins are detected using, for instance, a FANCJ, FANCD2, or FANCD1 binding agent, which in some instances will be detectably labeled. A binding agent binds substantially only to a defined target. Thus, a FANCD2 specific binding agent is an agent that binds substantially to a FANCD2 polypeptide. Similarly, a FANCJ binding agent is an agent that binds substantially to a FANCJ polypeptide and a FANCD1 specific binding agent is an agent that binds substantially to a FANCD1 polypeptide.

In certain embodiments, detecting a decrease in expression includes contacting a sample from the subject with a FANCJ, FANCD2, or FANCD1 binding agent, detecting whether the binding agent is bound by the sample, and thereby measuring the levels of FANCJ, FANCD2, or FANCD1 protein present in the sample. A decrease in the level of FANCJ, FANCD2, or FANCD1 protein in the sample, relative to a control indicates the subject has a predisposition to developing breast and/or ovarian cancer. In some examples, the control is the level of FANCJ, FANCD2, or FANCD1 protein found in an analogous sample from a subject not having breast and/or ovarian cancer, or a standard FANCJ, FANCD2, or FANCD1 protein level in analogous samples from a subject not having breast and/or ovarian cancer or not having a predisposition for developing breast and/or ovarian cancer. In some examples, the control is a statistical value, for example measured from multiple samples. In certain embodiments, the FANCJ, FANCD2, or FANCD1 binding agent is an antibody or an antibody fragment. In certain embodiments, the antibody is specific for the monoubiquinated form of FANCD2. In some embodiment, the binding agent binds functional forms of the protein but not non-functional forms of the protein.

In one embodiment, the specific binding agent is a monoclonal or polyclonal antibody that specifically binds the FANCD2 polypeptide. In another embodiment, the specific binding agent is a monoclonal or polyclonal antibody that specifically binds the FANCJ polypeptide. In yet another embodiment, the specific binding agent is a monoclonal or polyclonal antibody that specifically binds the FANCD1 polypeptide. In certain embodiments, the antibody is specific for a normal functional protein but not an abnormal non-functional protein.

The term “specifically binds” refers to, with respect to an antigen such as FANCD2, FANCD1, or FANCJ, the preferential association of an antibody or other ligand, in whole or part, with a cell or tissue bearing that antigen and not to cells or tissues lacking that antigen. It is recognized that a certain degree of non-specific interaction may occur between a molecule and a non-target cell or tissue. Nevertheless, specific binding can be distinguished as mediated through specific recognition of the antigen. Although selectively reactive antibodies bind antigen, they can do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time), for example to a cell or tissue bearing the FANCD2, FANCD1, or FANCJ polypeptide as compared to a cell or tissue lacking the polypeptide. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

### Lateral Flow Devices

It is contemplated by this disclosure that the immunoassays described above can be made in the form of a lateral follow device. Lateral flow devices of this disclosure can be prepared by conjugating a specific binding agent, such as an antibody, to a lateral flow substrate, such as a nitrocellulose lateral flow immunochromatographic strip. Such strips can contain specific binding agents that bind to, for example at least one of a FANCD1, FANCD2, and FANCJ protein. For example, the presence of specific binding agents for all three of FANCD1, FANCD2, and FANCJ in the same strip allows convenient rapid clinical testing of FANCD1, FANCD2, and FANCJ in the same biological specimen.

Disclosed herein are lateral flow devices for determining the presence and/or amount of at least one of a FANCD1, FANCD2, and FANCJ protein in a fluid sample. These devices typically include a sample application area and a separate FANCD1, FANCD2, or FANCJ protein capture area in which an immobilized one or more of a FANCD1, FANCD2, and FANCJ protein binding agent is provided which has a binding affinity for FANCD1, FANCD2, or FANCJ protein. Any liquid (such as a liquid biological sample) applied in the sample application area flows in a direction of flow from the sample application area to the FANCD1, FANCD2, or FANCJ protein capture area. Formation of a complex between the FANCD1, FANCD2, or FANCJ protein and the immobilized FANCD1, FANCD2, and FANCJ binding agent can be detected to determine the presence and/or amount of the FANCD1, FANCD2, or FANCJ protein in a fluid sample.

In some embodiments of the lateral flow device, a conjugate pad is placed in the path of flow from the sample application area to the FANCD1, FANCD2, or FANCJ protein capture area. The conjugate pad includes a mobile or mobilizable detector reagent for one or more of a FANCD1, FANCD2, and FANCJ protein, such that flow of liquid through the pad moves the detector reagent to the FANCD1, FANCD2, or FANCJ protein capture area. Formation of a complex among the detector reagent, FANCD1, FANCD2, or FANCJ protein and FANCD1, FANCD2, and FANCJ binding agent provides a visible or otherwise detectable indicator of the presence of FANCD1, FANCD2, and FANCJ protein in a biological specimen. In alternative embodiments, the detector reagent is not supplied in a conjugate pad, but is instead applied to the strip, for example from a developer bottle.

Examples of the detector reagent include one or more of an enzyme, colloidal gold particle, colored latex particle, protein-adsorbed silver particle, protein-adsorbed iron particle, protein-adsorbed copper particle, protein-adsorbed selenium particle, protein-adsorbed sulfur particle, protein-adsorbed tellurium particle, protein-adsorbed carbon particle, and protein-coupled dye sac.

The disclosed lateral flow devices can be used in methods for diagnosing a predisposition for developing breast and/or ovarian cancer in subject by analyzing a biological sample from the subject, by applying the biological sample to the device and detecting formation of a complex among the FANCD1, FANCD2, or FANCJ protein, the FANCD1, FANCD2, and FANCJ binding agent, and a detector reagent in the capture area. Detection of the formation of the complex in the capture area detects a FANCD1, FANCD2, or FANCJ protein. In those embodiments in which the device includes a conjugate pad in the path of flow from the sample application area to the FANCD1, FANCD2, or FANCJ protein capture area, the detected complex includes the mobile or mobilizable detector. In other embodiments in which the detector reagent is applied to the device from an external source, the detected complex includes the externally applied detector.

In general, a fluid sample (or a sample suspended in a fluid) is introduced to the strip at the proximal end of the strip, for instance by dipping or spotting. A sample is collected or obtained using methods well known to those skilled in the art. The sample may be diluted, purified, concentrated, filtered, dissolved, suspended, or otherwise manipulated prior to immunoassay to optimize the immunoassay results. The fluid migrates distally through all the functional regions of the strip. The final distribution of the fluid in the individual functional regions depends on the adsorptive capacity and the dimensions of the materials used.

The construction and design of lateral flow devices is very well known in the art, as described in the immediately preceding section, and see, for example, Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Lateral flow devices may have a wide variety of physical formats that are equally well known in the art. Any physical format that supports and/or houses the basic components of a lateral flow device in the proper function relationship is contemplated by this disclosure.

### Methods of Monitoring Disease in a Subject

The methods disclosed herein are particularly suited for monitoring disease progression in a subject, such as ovarian and/or breast cancer. In some embodiments, such methods involve detecting expression of at least one of a FANCD2, FANCJ, and FANCD1 molecule in a subject at a first time point, detecting expression of at least one of a FANCD2, FANCJ, and FANCD1 molecule in a subject at a second time point, and comparing the expression of at least one of a FANCD2, FANCJ, and FANCD1 molecules. If a decrease in the expression of at least one of a FANCD2, FANCJ, and FANCD1 molecule at the second time point is detected the subject is showing signs of disease progression. Conversely, if an increase in expression of at least one of a FANCD2, FANCJ, and FANCD1 molecules is observed at the second time point the subject is showing signs of disease remission. In some embodiments, methods of monitoring disease progression in a subject involve detecting the number of radial formations and/or chromosomal breakages is a cell obtained from a subject induced by a DNA damaging agent at a first time point and comparing the number of radial formations and/or chromosomal breakages is a cell obtained from a subject induced by a DNA damaging agent at a second time point. If an increase in the number of radial formations and/or chromosomal breakages at the second time point is detected the subject is showing signs of disease progression. Conversely, if a decrease in the number of radial formations and/or chromosomal breakages at the second time point is observed the subject is showing signs of disease remission.

Also encompassed by this disclosure are methods for selecting a treatment regimen or therapy for the prevention, reduction, or inhibition of ovarian and/or breast cancer. In some examples, these methods involve detecting a decrease in expression of at least one of a FANCD2, FANCJ, and FANCD1 molecule in a subject, and if such decrease is detected, a treatment is selected to prevent or reduce ovarian and/or breast cancer or to delay the onset of ovarian and/or breast cancer. In some examples these methods involve detecting a increase in the number of radial formations and/or chromosomal breakages, and if such increase is detected, a treatment is selected to prevent or reduce ovarian and/or breast cancer or to delay the onset of ovarian and/or breast cancer. The subject then can be treated in accordance with this selection. Such treatments include without limitation the use of chemotherapeutic agents, immunotherapeutic agents, radiotherapy, surgical intervention, or combinations thereof.

In the case of women with ovarian or breast cancer in remission whose normal epithelial cells score as genetically unstable in the MMC test, confirmation of responses to preventive agents (that increase expression of a NNC FA component gene product), can be performed, for example by using ELISA, immunohistochemistry, immunoblotting, or real-time RT-PCR assays.

### Kits

This disclosure provides for kits using the methods disclosed herein. Kits for measuring expression of FANCD2, FANCJ, and FANCD1 molecules, can include a binding molecule that selectively binds to FANCD2, FANCJ, or FANCD1 molecules. In some examples of such kits where FANCD2, FANCJ, or FANCD1 is a FANCD2, FANCJ, or FANCD1 protein, the binding molecule provided in the kit can be an antibody or antibody fragment that selectively binds to the FANCD2, FANCJ, or FANCD1 protein. In other examples of such kits where FANCD2, FANCJ, or FANCD1 is a FANCD2, FANCJ, or FANCD1 nucleic acid, the binding molecule provided in the kit can be an oligonucleotide capable of hybridizing to the FANCD2, FANCJ, or FANCD1 nucleic acid molecule.

Kits are also provided that contain the necessary reagents for determining gene copy number (genomic amplification or deletion), such as probes or primers specific for FANCD2, FANCD1, or FANCJ nucleic acid sequence. These kits can each include instructions, for instance instructions that provide calibration curves or charts to compare with the determined (e.g., experimentally measured) values. Kits are also provided for determining the number of radial formations and/or chromosomal breakages in response to a DNA damaging agent.

Kits for Detection of mRNA Expression

The nucleotide sequence of FANCD2, FANCD1, and/or FANCJ nucleic acid molecules, and fragments thereof, can be supplied in the form of a kit for use in detection of expression of FANCD2, FANCD1, or FANCJ and/or diagnosis of progression to or predisposition to ovarian and/or breast cancer. In such a kit, an appropriate amount of one or more oligonucleotide primer specific for FANCD2, FANCD1, or FANCJ is provided in one or more containers. The oligonucleotide primers can be provided suspended in an aqueous solution or as a freeze-dried or lyophilized powder, for instance. The container(s) in which the oligonucleotide(s) are supplied can be any conventional container that is capable of holding the supplied form, for instance, microfuge tubes, ampoules, or bottles. In some applications, pairs of primers can be provided in pre-measured single use amounts in individual, typically disposable, tubes, or equivalent containers. With such an arrangement, the sample to be tested for the presence of ovarian and/or breast cancer-related genomic amplification/deletion can be added to the individual tubes and in vitro amplification carried out directly.

The amount of each oligonucleotide primer supplied in the kit can be any amount, depending for instance on the market to which the product is directed. For instance, if the kit were adapted for research or clinical use, the amount of each oligonucleotide primer provided likely would be an amount sufficient to prime several in vitro amplification reactions. Those of ordinary skill in the art know the amount of oligonucleotide primer that is appropriate for use in a single amplification reaction. General guidelines can for instance be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, N.Y., 1989), and Ausubel et al. (In Current Protocols in Molecular Biology, John Wiley & Sons, New York, 1998).

In some embodiments, kits can also include the reagents necessary to carry out in vitro amplification reactions, including, for instance, DNA sample preparation reagents, appropriate buffers (for example polymerase buffer), salts (for example magnesium chloride), and deoxyribonucleotides (dNTPs). Written instructions can also be included.

Kits can include either labeled or unlabeled oligonucleotide probes for use in detection of the in vitro amplified sequences. The appropriate sequences for such a probe will be any sequence that falls between the annealing sites of two provided oligonucleotide primers, such that the sequence the probe is complementary to is amplified during the in vitro amplification reaction (if it is present in the sample).

It may also be advantageous to provide in the kit one or more control sequences for use in the in vitro amplification reactions. The design of appropriate positive control sequences is well known to one of ordinary skill in the appropriate art.

In some embodiments, kits for detection of ovarian and/or breast cancer-related mRNA expression can also include reagents necessary to carry out RT-PCR or other in vitro amplification reactions, including, for instance, RNA sample preparation reagents (including for example an RNAse inhibitor), appropriate buffers (for example polymerase buffer), salts (for example magnesium chloride), and deoxyribonucleotides (dNTPs). Written instructions can also be included.

Alternatively, kits can be provided with the necessary reagents to carry out quantitative or semi-quantitative Northern analysis of FANCD2, FANCD1, or FANCJ mRNA. Such kits include, for instance, at least one ovarian and/or breast cancer-related sequence-specific oligonucleotide for use as a probe. This oligonucleotide can be labeled in any conventional way, including with a selected radioactive isotope, enzyme substrate, co-factor, ligand, chemiluminescent or fluorescent agent, hapten, or enzyme.

Kits for Detection of Ovarian and/or Breast Cancer-Linked Protein or Peptide Expression

Kits for the detection of FANCD2, FANCD1, or FANCJ protein expression are also encompassed herein. Such kits can include for example at least one target protein specific binding agent (for example a polyclonal or monoclonal antibody or antibody fragment), and optionally can include at least one control. The ovarian FANCD1, FANCD2, or FANCJ protein specific binding agent and control can be contained in separate containers. The kits can also include methods for detecting FANCD1, FANCD2, or FANCJ protein:agent complexes, for instance the agent can be detectably labeled. If the detectable agent is not labeled, it can be detected by second antibodies or protein A, for example, either of both of which also can be provided in some kits in one or more separate containers. Such techniques are well known to those of ordinary skill in the art. In certain embodiments, these kits can include the lateral flow devices of the present disclosure.

Additional components in some kits include instructions for carrying out the assay. Instructions will allow the tester to determine whether FANCD1, FANCD2, or FANCJ expression levels are elevated or reduced in comparison to a control sample. Reaction vessels and auxiliary reagents such as chromogens, buffers, enzymes, etc. also may be included in the kits.

### Kits for Detections of Chromosomal Breakage and Radial Formation

Kits for Detecting chromosomal breakage and radial formation in response to a DNA damaging agent are also encompassed by this disclosure. Such kits can include a DNA damaging agent, such as MMC or DEB. The kits also can contain agents to arrest cells in metaphase. Agents useful in arresting cells in metaphase include colcemid. The kits may also contain the culture media for cell growth. Kits may contain other reagents such as phytohemagglutinin, and growth factors. Additional components in some kits include instructions for carrying out the assay. Instructions will allow the tester to determine radial formation and chromosomal breakage is elevated or reduced in comparison to a control sample. Reaction vessels and auxiliary reagents also may be included in the kits.

### Identification of Therapeutic Compounds

Decreases in the activity of FA NNC component associated with breast and/or ovarian cancer, can be used to identify compounds that are useful in treating, reducing, or preventing ovarian and/or breast cancer or development or progression of ovarian and/or breast cancer. The methods for identifying compounds useful for treating such cancers involves determining if application of a test compound increases expression of FANCD2, FANCJ, or FANCD1, and selecting a compound that increases expression of FANCD2, FANCJ, or FANCD1. Alternatively, a compound can be selected that reduces the number of radial formations and/or chromosomal breakages.

The disclosed methods are suitable for screening large libraries of compositions to identify compounds that are useful in treating and/or inhibiting (including preventing) the development of breast and/or ovarian cancer. Examples of disclosed methods involve contacting test cells with a test compound, then measuring expression of at least one of FANCD2, FANCJ, or FANCD1 in the test cells. In such methods, a increase in expression of at least one of FANCD2, FANCJ, or FANCD1 relative to the expression of FANCD2, FANCJ, or FANCD1 in control, such as cells not contacted with the test compound, indicates that the test compound is useful in treating, reducing, or preventing ovarian and/or breast cancer or development or progression of ovarian and/or breast cancer.

Measuring the expression of FANCD2, FANCJ, or FANCD1 can involve detecting the expression of FANCD2, FANCJ, or FANCD1 in the test cell after contacting the cell with the test compound, and comparing the test cell expression of FANCD2, FANCJ, or FANCD1 to the expression of FANCD2, FANCJ, or FANCD1 in at least one control cell. Representative control cells include cells taken from breast and/or ovarian tissue ovarian epithelial cancer tissue, ovarian epithelial tumors, ovarian germ cell tumors, stromal tumors, and ovarian and/or breast cancer tissues in any progressive stage (for example, stage I-IV ovarian cancer). It is understood that any technique can be used to detect the activity of the FA NNC component.

By way of example, a test compound is applied to a cell, for instance a test cell, which is monitored for expression or activity of one or more of members of the FA NNC component. Expression in the contacted test cell is compared to the equivalent measurement from a test cell in the absence of the test compound. Compounds that alter activity of the FA NNC component (for instance as detected by increasing expression of FANCD2, FANCD1, or FANCJ gene product or by reducing radial formation and/or chromosomal breakages) are selected as a likely candidates for further characterization to determine toxicity, bioavailability, stability, and the like. Additionally, the activity of the selected compound to inhibit growth of ovarian and/or breast cancer is typically evaluated in vitro and/or in vivo to confirm biological activity. Such identified compounds are useful in treating, reducing, or preventing ovarian and/or breast cancer or development or progression of ovarian and/or breast cancer.

### Exemplary Test Agents

An “agent” is any substance or any combination of substances that is useful for achieving an end or result. The agents identified using the methods disclosed herein can be of use for treating and/or preventing cancer, such as breast and/or ovarian cancer. Any agent that has potential (whether or not ultimately realized) affect the activity of the FA NNC component can be tested using the methods of this disclosure.

Exemplary agents include, but are not limited to, peptides such as, soluble peptides, including but not limited to members of random peptide libraries (see, for example Lam et al., Nature, 354:82-84, 1991; Houghten et al., Nature, 354:84-86, 1991), and combinatorial chemistry-derived molecular library made of D-and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids. In some examples, a test agent is a known anti-neoplastic agent.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produce in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial chemical or peptide library containing a large number of potential therapeutic compounds. Such combinatorial libraries are then screened in one or more assays as described herein to identify those library members (particularly chemical species or subclasses) that display a desired characteristic activity (such as, increasing the activity of the FA NNC component), for example by increasing the expression of one or more of FANCD2, FANCD1, and FANCJ.

The compounds identified using the methods disclosed herein can serve as conventional “lead compounds” or can themselves be used as potential or actual therapeutics. In some instances, pools of candidate agents may be identify and further screened to determine which individual or subpools of agents in the collective have a desired activity.

### Gene Therapy

Gene therapy approaches for combating cancer (particularly ovarian and breast cancer) in subjects are made possible by the present disclosure.

Such approaches involve selection of a subject with decreased expression of a FA NNC component protein such as FANCD1, FANCD2, or FANCJ and expressing in the subject a recombinant genetic construct that includes an nucleic acid encoding of one or more of FANCD2, FANCJ, and FANCD1 operably linked to a promoter, wherein expression of the nucleic acid molecule increases expression of FANCD2, FANCJ, and/or FANCD1.

It will also recognized by those of ordinary skill in the art that that nucleic acids encoding the FANCD1, FANCD2, and FANCJ polypeptides include degenerate variants by virtue of the genetic code. There are 20 natural amino acids, most of which are specified by more than one codon. Therefore, all degenerate nucleotide sequences are included as long as the amino acid sequence of polypeptide encoded by the nucleotide sequence is unchanged (for example the FANCD2 polypeptide). In some embodiments, the coding region can be altered by taking advantage of the degeneracy of the genetic code to alter the coding sequence such that, while the nucleotide sequence is substantially altered, it nevertheless encodes a protein having an amino acid sequence substantially similar to the human FANCD2, FANCD1, or FANCJ protein sequences. For example, because of the degeneracy of the genetic code, four nucleotide codon triplets—(GCT, GCG, GCC and GCA)—code for alanine. The coding sequence of any specific alanine residue within the human FANCD2 protein, therefore, could be changed to any of these alternative codons without affecting the amino acid composition or characteristics of the encoded protein. Based upon the degeneracy of the genetic code, variant DNA molecules can be derived from the cDNA and gene sequences using standard DNA mutagenesis techniques, or by synthesis of DNA sequences.

Typically a vector will include nucleic acid sequences that permit it to replicate in a host cell, such as an origin of replication or expression control sequences. Expression control sequences are nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (typically ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences, and fusion partner sequences. Expression control sequences can include a promoter. A promoter is an array of nucleic acid control sequences that directs transcription of a nucleic acid. A promoter includes necessary nucleic acid sequences near the start site of transcription, for example, in the case of a polymerase II type promoter, a TATA element. A promoter also optionally includes distal enhancer or repressor elements, which can be located as much as several thousand base pairs from the start site of transcription. Both constitutive and inducible promoters are included (see for example, Bitter et al., Methods in Enzymology 153:516-544, 1987). A vector may also include one or more selectable marker genes and other genetic elements known in the art. When introduced into a host cell a vector produces a transduced host cell. Host cells are cells in which a nucleic acid is introduced and optionally expressed. The cell can be prokaryotic or eukaryotic. Host cells also include cells of a subject transduced with a vector. The term host cell also includes any progeny of the host cell in which the nucleic acid was introduced. It is understood that all progeny may not be identical to the parental cell since there may be mutations that occur during replication. However, such progeny are included when the term “host cell” is used.

Retroviruses have been considered a preferred vector for in gene therapy, with a high efficiency of infection and stable integration and expression (see Orkin et al., Prog. Med. Genet. 7:130-142, 1988). A full-length FA NNC component gene or cDNA (such as FANCD2, FANCJ, or FANCD1) can be cloned into a retroviral vector and driven from either its endogenous promoter or from the retroviral LTR (long terminal repeat). Other viral transfection systems can also be utilized for this type of approach, including adenovirus, adeno-associated virus (AAV) (see McLaughlin et al., J. Virol. 62:1963-1973, 1988), Vaccinia virus (Moss et al., Annu. Rev. Immunol. 5:305-324, 1987), Bovine Papilloma virus (Rasmussen et al., Methods Enzymol. 139:642-654, 1987) or members of the herpesvirus group such as Epstein-Barr virus (Margolskee et al., Mol. Cell. Biol. 8:2837 2847, 1988).

In addition to delivery of FANCD2, FANCD1, or FANCJ protein encoding sequences to cells using viral vectors, it is possible to use non-infectious methods of delivery. For instance, lipidic and liposome-mediated gene delivery has recently been used successfully for transfection with various genes (for reviews, see Templeton and Lasic, Mol. Biotechnol. 11:175-180, 1999; Lee and Huang, Crit. Rev. Ther. Drug Carrier Syst. 14:173-206; and Cooper, Semin. Oncol. 23:172-187, 1996). For instance, cationic liposomes have been analyzed for their ability to transfect monocytic leukemia cells, and shown to be a viable alternative to using viral vectors (de Lima et al., Mol. Membr. Biol. 16:103-109, 1999). Such cationic liposomes can also be targeted to specific cells through the inclusion of, for instance, monoclonal antibodies or other appropriate targeting ligands (see Kao et al., Cancer Gene Ther. 3:250-256, 1996).

Developments in gene therapy techniques include the use of RNA-DNA hybrid oligonucleotides, as described by Cole-Strauss et al. (Science 273:1386-1389, 1996). This technique may allow for site-specific integration of cloned sequences, thereby permitting accurately targeted gene replacement.

The following examples are provided to illustrate certain particular features and/or embodiments. These examples should not be construed to limit the invention to the particular features or embodiments described.

### Examples

### Example 1

### Selection of Tissues and Culture Conditions

Ovarian tissue sample were obtained from 1) subjects with a family history of ovarian and/or breast cancer; 2) subjects with ovarian cancer; and 3) normal subjects with neither a diagnosis nor a family history of ovarian and/or breast cancer.

Subjects at high risk for ovarian cancer were defined as women with i) a family history of one or more 1st degree relatives diagnosed with ovarian cancer prior to the age of 50 years, ii) a family history of one 1st degree relative with ovarian cancer and one or more 1st or 2nd degree relatives diagnosed with breast or ovarian cancer, or iii) a personal history of breast cancer and one or more 1st or 2nd degree relatives diagnosed with breast or ovarian cancer. These criteria have been previously validated as risk factors for ovarian and breast cancer (U.S. Preventive Services Task Force recommendations. Summaries for patients. Genetic risk assessment and BRCA mutation testing for breast and ovarian cancer susceptibility. Ann. Intern. Med. 2005;143:147). None of the subjects had received any cytotoxic chemotherapy or radiation prior to surgery.

A total of 25 samples were obtained including normal ovarian tissue (11 samples from 9 subjects), high-risk ovarian tissue (6 samples from 5 subjects), and ovarian cancer samples (8 samples from 8 subjects) and analyzed as described in the following examples.

TABLE 1
Clinico-pathologic and molecular characteristics of 22 cases studied.

| | | | Radials (%) | | |
|---|---|---|---|---|---|
| | | | OSE | PBML | FANCD2 |
| | | | MMC/ | MMC/ | Expression |
| Cases | Age | Pathology | DEB | DEB | (OSE) |
| Normal | | | | | |
| OV-NL 1 | 62 | ROV: no abnormalities | 0/0 | N.D. | Normal |
| | | LOV: endometriosis | | | |
| OV-NL 2 | 51 | Uterine fibroids. Normal ovaries. | 0/0 | N.D. | Normal |
| OV-NL 3 | 40 | Metrorrhagia. Normal ovaries. | 2/0 | 0/0 | Normal |
| OV-NL 4 | 54 | LOV: No abnormalities. | 4/1 | N.D. | Normal |
| | | ROV: Benign serous | | | |
| | | cystadenoma. | | | |
| OV-NL 5 | 68 | LOV: Fibroma. | 1/1 | N.D. | Normal |
| | | ROV: No abnormalities. | | | |
| OV-NL 6 L | 43 | CIN III. Normal ovaries. | 0/0 | 0/N.D. | Normal |
| OV-NL 6 R | | | 0/0 | 0/N.D. | |
| OV-NL 7 | 54 | LOV: Benign serous | 1/2 | N.D. | Normal |
| | | cystadenoma. | | | |
| | | ROV: no abnormalities | | | |
| OV-NL 8 | 55 | Uterine polyp. Normal ovaries. | 3/3 | N.D. | Normal |
| OV-NL 9 L | 48 | Uterine fibroid. Normal ovaries. | 0/0 | 0/0 | Normal |
| OV-NL 9 R | | | 0/0 | 0/0 | |
| High-Risk | | | | | |
| OV-HR 1 | 30 | No evidence of multifocal | 10/14 | 0/0 | Normal |
| OV-HR 2 | 71 | surface papillomatosis, | 50/64 | 0/0 | Reduced |
| OV-HR 3 | 43 | pseudostratification or activity of | 64/55 | 1/0 | Normal |
| OV-HR 4 L | 35 | the epithelium or ovarian stroma. | 66/50 | 0/0 | Reduced |
| OV-HR 5 L | 71 | | 47/20 | 0/1 | Reduced |
| OV-HR 5 R | | | 30/12 | 0/0 | Reduced |
| Carcinoma | | | | | |
| OV-CA 1 | 72 | Poorly differentiated serous | 14/16 | 2/0 | Normal |
| | | carcinoma, stage III C | | | |
| OV-CA 2 | 52 | Poorly differentiated serous | 12/10 | 1/0 | Normal |
| | | carcinoma, stage IV | | | |
| OV-CA 3 | 63 | Moderately differentiated serous | 16/14 | 1/1 | Normal |
| | | carcinoma, stage IIIB | | | |
| OV-CA 4 | 70 | Poorly differentiated serous | 70/48 | 4/2 | Reduced |
| | | carcinoma, stage III C | | | |
| OV-CA 5 | 54 | Poorly differentiated serous | 12/2 | 2/0 | Normal |
| | | carcinoma, stage III C | | | |
| OV-CA 6 | 73 | Poorly differentiated serous | 66/N.D. | 1/1 | Normal |
| | | carcinoma, stage IV | | | |
| OV-CA 7 | 58 | Poorly differentiated serous | 0/N.D. | 0/1 | Normal |
| | | carcinoma, stage III C | | | |
| OV-CA 8 | 62 | Poorly differentiated serous | 33/N.D. | 3/1 | Normal |
| | | carcinoma, stage III C | | | |

Abbreviations;
OSE, ovarian surface epithelium;
PBML: peripheral blood mono-lymphocytes;
LOV, left ovary;
ROV, right ovary;
CIN III, cervical intraepithelial dysplasia III;
N.D., not determined.

Peripheral blood was obtained from all subjects. Lymphocytes were isolated using Ficoll-Paque™ PLUS (Amersham Biosciences, Piscataway, N.J.), then stimulated with 1% phytohemagglutinin (PHA) with and without MMC for 4 days before harvest. Harvested lymphocytes were used to prepare cell lysates for chromosomal breakage analysis, and for in vitro MMC survival assays.

The ovarian cells were scraped from the ovarian surface and enzymatically disaggregated with Collagenase I (GIBCO-Invitrogen, Grand Island, N.Y.) for 4 hours. The cells were washed in RPMI 1640 medium (GIBCO-Invitrogen) and plated in 25 cm² flasks coated with collagen in RPMI 1640 supplemented with 20% FCS (Hyclone, Logan, Utah), 10 μg/ml insulin (Sigma, St. Louis, Mo.) and 10 ng/ml EGF (R&D Systems, Minneapolis, Minn.). Studies were performed on primary cells and immortalized cells. Ovarian cells were immortalized by transduction with a retrovirus expressing SV40 large T-antigen obtained from cell line Ψ-2/U195 (Saito et al., Mutat. Res. 294:255-62, 1993; Williams et al., Mol. Cell Bio. 8:3864-71, 1988). SV40-transformed ovarian epithelial cells were transduced with pMMP retroviral vectors containing full-length FANCD2 cDNA produced from the AM12/RVD2 cell line (Naf et al., Mol. Cell Biol. 18:5952-60, 1998; Kuang et al., Blood 96:1625-32, 2000).

The ovarian cancer cell lines PA1 and OVCAR-3 and the cervical epithelial cell line HeLa (American Type Culture Collection, Manassas, Va.) were also used for p53 studies designed to quantify the function of p53. RNA was obtained from these three cell types and the primary cells OV-HR3, OV-HR4L, and OV-HR5R (Table 1) before and 18 hours after exposure to 20 J/m2 UV radiation. RNA was used in real-time reverse transcription-PCR (RT-PCR) to detect fold changes in three p53-responsive genes: p21, Noxa, and Puma.

### Example 2

### Sensitivity DNA Damaging Agents

Reduced viability following treatment with agents that induce DNA damage, such as DNA crosslinking agents, is an indicator or reduced activity of the FA NNC component.

The ability of ovarian epithelial cells obtained from high risk subjects; subjects with ovarian cancer; and normal subjects to withstand exposure to the DNA alkylating agent MMC was therefore evaluated.

Epithelial cells (6×10³) were incubated with various concentrations of MMC (range 0 to 250 nM) in 12-well plates, in RPMI 1640 medium with 15% FCS, 100 units/ml penicillin/streptomycin, and 2 mM L-glutamine. After a 5 day incubation, cells in the monolayer were trypsinized, and live cells were counted using the trypan blue dye exclusion method. Cell viability was expressed as percentage of trypan blue-excluding cells in the MMC-treated sample relative to that an untreated control sample. Each sample was analyzed in triplicate.

### Example 3

### MMC-Induced Chromosomal Breakage

Chromosomal breakage and radial formation in response to DNA damaging agents was also evaluated in these samples.

For breakage studies, cell cultures were incubated with 40 ng/ml MMC and 200 ng/ml diepoxybutane (DEB) at 37° C. for 48 hours in RPMI 1640 medium in the dark. These cultures were then harvested after a 2 hour exposure to 0.25 μg/ml Colcemid (Sigma). Following a 10 minute treatment with hypotonic solution (0.075 M KCl, 5% fetal calf serum) the cells were fixed with a 3:1 mixture of methanol:acetic acid. Slides were stained with Wright's stain, and breaks and radial formation was assessed by counting the number of breaks and radials in a representative number of cells.

The normal range of MMC and DEB-induced chromosome radial formation has been well established using fibroblasts and lymphoid cells, but the range of normal responses has not been defined for primary cultures of normal ovarian epithelial cells. The epithelial cells from all normal ovarian samples showed levels of MMC- and DEB-induced radial formation consistent with the range defined for other well-studied normal cell types (<20% metaphases). However, 5 of 6 high-risk ovarian samples and 3 of 8 ovarian cancer samples had increased levels of chromosome breakage and radial formation (Table 1). None of the subjects exhibited increased chromosome breakages in peripheral blood lymphocytes treated with MMC/DEB, confirming that none of the subject had Fanconi anemia and demonstrating that the loss of activity of the FA NNC component was specific to the female reproductive tissue (Table 1).

### Example 4

### Analysis of FANCD2 Expression

To determine whether decreased activity of the FA NNC component correlated with a decrease in FANCD2 expression, immunoblots were performed on ovarian epithelial cell samples.

All 25 primary ovarian epithelial cell cultures detailed in table 1 were screened for the presence of FANCD2 long (L) and short (S) forms by immunoblotting (Hussain et al., Human Mol. Genet. 13:1241-8, 2004).

In brief, 1×10⁶ cells were treated in vitro with or without 50 nM MMC for 48 hours. Whole-cell extracts were prepared in lysis buffer (50 mM Tris-HCl, pH 7.4, 150 mM NaCl, 1% NP-40, 0.1% sodium deoxycholate, 4 mM EDTA) supplemented with protease inhibitors (1 μg/ml leupeptin and pepstatin A, 2 mg/ml aprotinin, and 1 mM phenylmethylsulfonyl fluoride) and phosphatase inhibitors (2 mM sodium orthovanadate and 10 mM sodium fluoride). 100 μg cell lysates were boiled 5 min in 1× Laemmli buffer (2% SDS, 20% Glycerol, 0.5 M Tris-HCL (pH 6.8), 100 nM β-Mercaptoethanol), electrophoresed on a 7.5% polyacrylamide SDS gel, and then transferred to nitrocellulose membranes. After blocking with 5% nonfat dried milk in TBS-T (10 mM Tris, 150 mM NaCl (pH8.0), 0.1% Tween 20), the membrane was incubated overnight at 4° C. with anti-FANCD2 mouse monoclonal antibody (Santa Cruz Biotechnology, Santa Cruz, Calif., diluted 1:200 in TBS-T), anti-Tubulin antibodies (Sigma, diluted 1:150 in TBS-T), anti-p53 (CalBiochem, San Diego, Calif., diluted 1:1,000 in TBS-T), and anti-beta-actin (Santa Cruz Biotechnology, Santa Cruz, Calif., diluted 1:500 in TBS-T). Blots were then washed with TBS-T and incubated for 1 hour with a 1:10,000 dilution of horseradish peroxidase-conjugated goat anti-mouse secondary antibody or goat anti-rabit secondary antibody (Bio-Rad, Hercules, Calif.) at room temperature. Binding was detected using Super Signal West Pico Chemiluminescence substrate (Pierce Biotechnology, Rockford, Ill.).

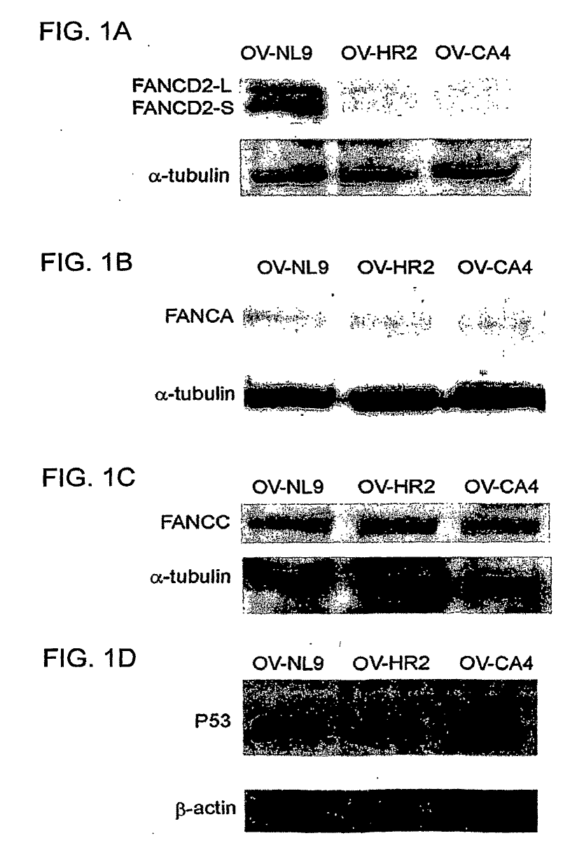

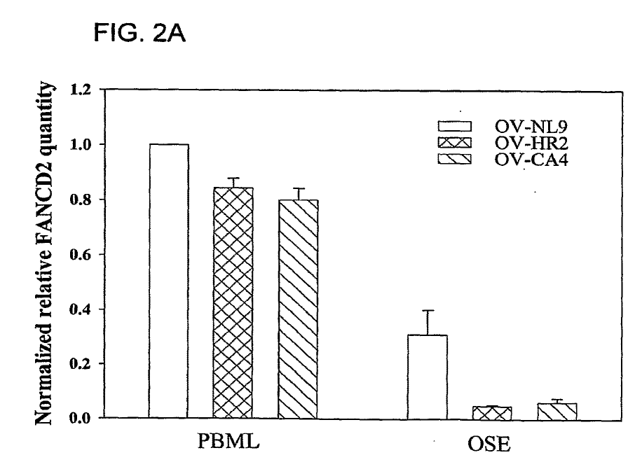

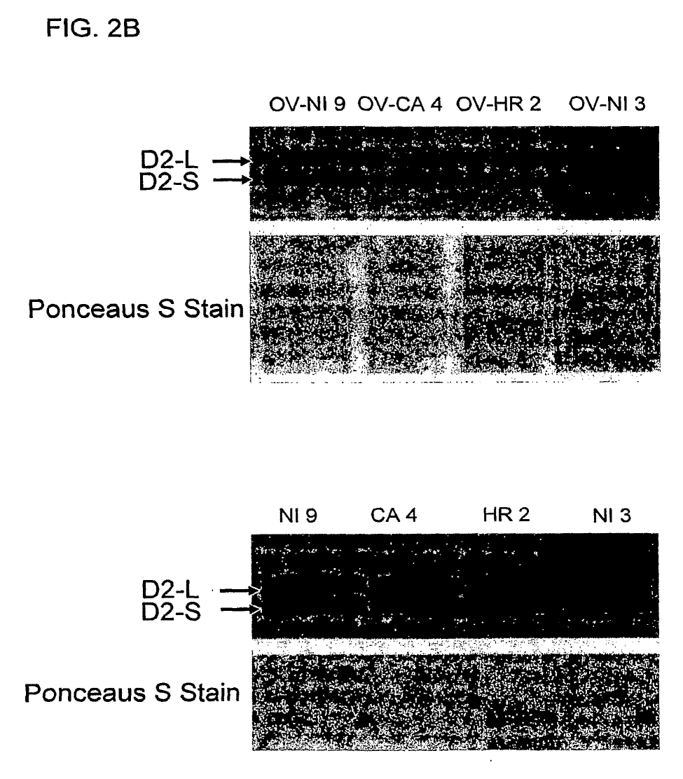

Reduced levels of FANCD2 protein (both the L- and S-forms) were consistently found in 4 of the 6 high-risk, and one of the 3 breakage positive ovarian cancer samples. FANCD2 levels were never reduced in cells that were resistant to alkylating agents in the chromosomal breakage test. Both primary cells and cell lines created by SV40 transformation of two of these samples, designated OV-HR2 (from a cancer-free, high-risk subject) and OV-CA4 (from ovarian cancer cells), both showed markedly reduced levels of FANCD2-L and FANCD2-S protein isoforms, as compared to normal control (FIG. 1A). However, other proteins involved in pathways of DNA damage response, including FANCA (see FIG. 1B) and FANCC (see FIG. 1C), showed no such reduction in levels in the high-risk or ovarian cancer cells compared with normal control (FIG. 1A). Immunoblotting with anti-p53 antibody revealed that full-length protein was present in all samples, ruling out large genomic deletions of this gene that might be expected in cells with chromosomal instability (FIG. 1D). Oligonucleotide array CGH experiments also ruled out p53 deletion, with no genomic loss of chromosome band 17p13.1 found. Additionally, it was determined that p53 function was normal in three of the primary cells tested (OV-HR3, OV-HR4L, and OV-HR5R). As a control, normal p53 function (PA1 cells) or loss of function (OVCAR-3 and HeLa) was confirmed and the FANCD2 levels were normal in the three lines. Thus, FANCD2 gene expression is not controlled by p53 (see Table 6). In contrast, FANCD2 mRNA (FIG. 2A) and protein (FIG. 2B) were readily detectable in PHA-stimulated peripheral blood lymphocytes from the same subjects.

TABLE 6
Functional Status of p53.

| | CELL LINES | | | PRIMARY CELLS | | |
|---|---|---|---|---|---|---|
| Protein | PA1 | HeLa | OVCAR-3 | OV-HR3 | OVHR4L | OVHR5R |
| p53 | Normal | Inactivated | Inactivated | Normal | Normal | Normal |
| FANCD2 | Normal | Normal | Normal | Normal | Reduced | Reduced |

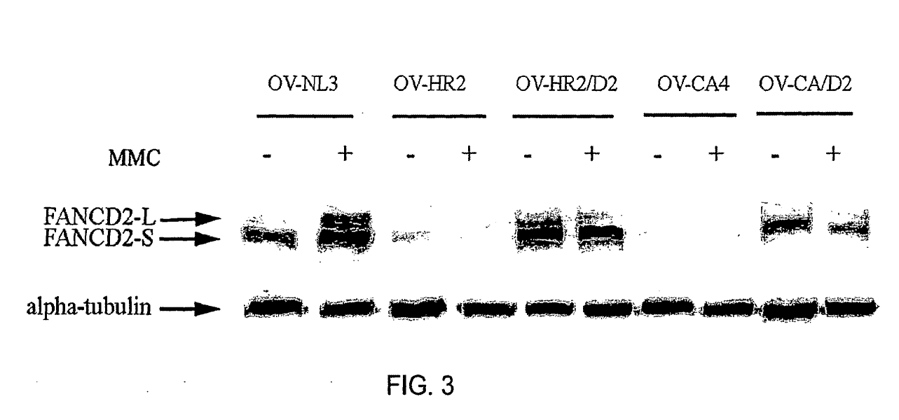

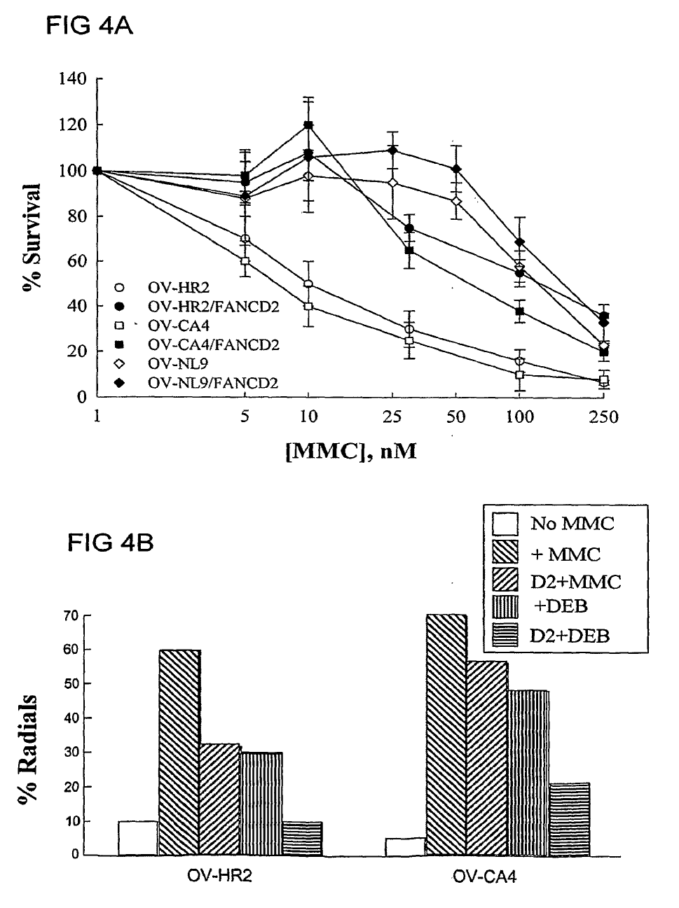

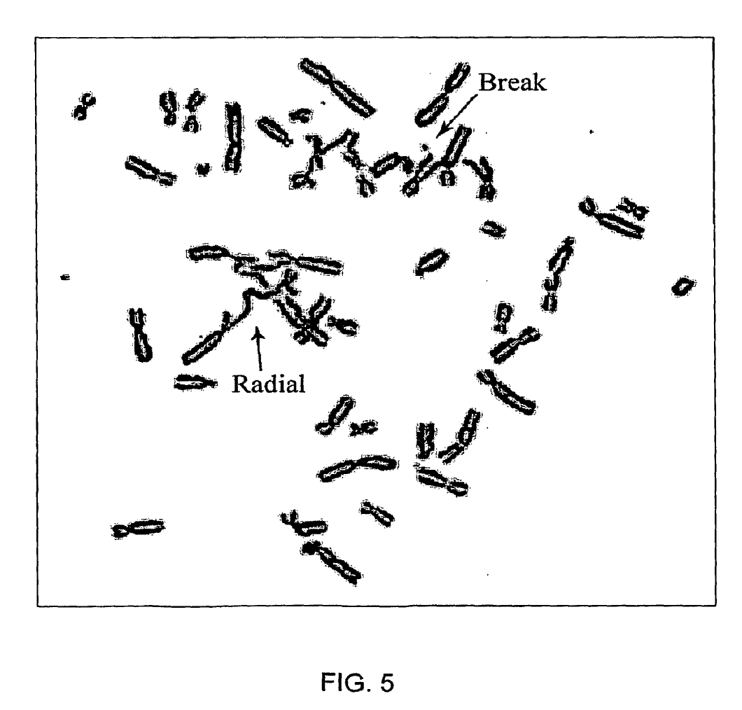

The FANCD2 deficiency as a causative agent in the genetic instability of OV-HR2 and OV-CA4 cells was confirmed by transducing these cells with pMMP retrovirus containing the FANCD2 cDNA. In both cases, normal FANCD2 levels were restored, and the cells responded normally to cross-linker exposure as demonstrated by increased levels of FANCD2-L after treatment with MMC (FIG. 3). FANCD2-deficient OV-HR2 and OV-CA4 cells show impaired survival even at low doses of MMC (FIG. 4A, LD₅₀=5-10 nM) but survival was greatly increased after transduction of the cells with FANCD2 retrovirus (LD₅₀=100 nM). Expression of FANCD2 also significantly reduced the fraction of MMC- and DEB-exposed cells with radial forms, from 60% to 32% (MMC) and 30% to 10% (DEB) in OV-HR2, and from 70% to 56% (MMC) and 48% to 20% (DEB) in OV-CA4 (FIG. 4B).

### Example 5

### Amplification and Sequencing of FANCD2 mRNA and DNA

To confirm that FANCD2 did not contain a mutation responsible for the reduced activity of the FA NNC component in ovarian epithelial tissues, FANCD2 was amplified and sequenced.

Total RNA was prepared from cultured ovarian epithelial cells using the RNeasy Mini kit (QIAGEN®, Inc., Valencia, Calif., USA). First-strand cDNA was synthesized using 2.0 μg RNA, 200 ng random hexamers (INVITROGEN™, Carlsbad, Calif.), and SUPERSCRIPT™ III reverse transcriptase (INVITROGEN™), according to manufacturer's instructions. PCR of full-length FANCD2 coding sequences was then performed with 2.0 μl cDNA, primers Xho-D2-1(5′-AGCTCGAGATGGTTTCCAAAAGAAGACTGTCAAAA-3′ (SEQ ID NO: 9) and Not-D2-4411 (5′-ATTGCGGCCGCCTAATCAGAGTCATCATAACTCTC-3′ (SEQ ID NO: 10), and PFUULTRA™ polymerase (STRATAGENE®, La Jolla, Calif., USA) according to manufacturer's instructions. PCR products were cloned using the pCR-Blunt II-TOPO system (INVITROGEN™), and cDNA inserts from individual clones were sequenced with the use of the Big Dye Terminator v.3.1 Cycle Sequencing Kit and an ABI PRISM® 3100 Genetic Analyzer (Applied Biosystems, Foster City, Calif.). Sequencing primers were chosen with a 200 base pairs (bp) reading overlap to insure full coverage. A cDNA insert containing the ex16-18del splice variant was subcloned into the retroviral vector pLXSN.

PCR amplification to confirm the presence of the ex15-17del splice variant, lacking exons 15 through 17, was performed on 2.0 μl cDNA using primers designed to bind within exons 13 and 19 of FANCD2 (upstream primer sequence 5′-CAAGAAAGCAGCGGTCAGAG-3′ (SEQ ID NO: 11); downstream primer sequence 5′-ACAGCACCAATAATCCCAATG-3′ (SEQ ID NO: 12)). PCR products were electrophoresed on a 1% agarose gel. Genomic DNA was isolated from ovarian cells using the QLAAMP® DNA Mini Kit (QIAGEN® Inc). FANCD2 exon-intron boundaries were amplified by PCR using 50 ng genomic DNA as template and one unit Taq DNA polymerase (Promega, Madison, Wis.). PCR products were sequenced as indicated above.

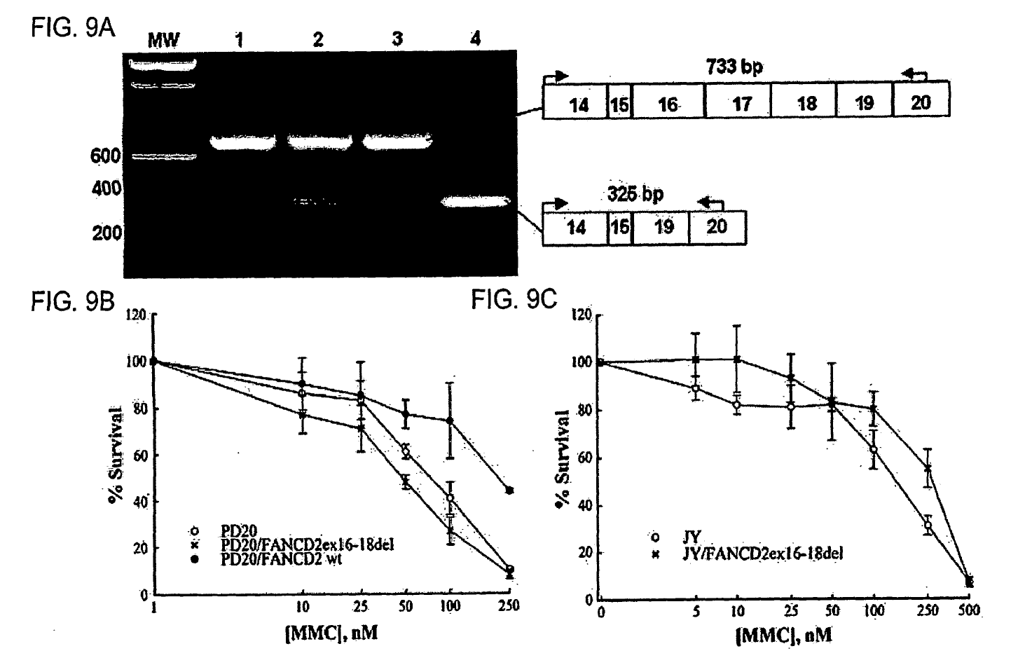

Cloning and sequencing of the FANCD2 cDNA samples from OV-HR2 and OV-CA4 revealed the existence of two transcripts, each found in multiple clones: one, the full-length wild-type sequence, and the other, a differentially spliced form showing a deletion of exons 15-17. Deletion of exons 15-17 was confirmed using RT-PCR with primers designed to bind specifically within exons 13 and 19 of FANCD2. PCR products were analyzed by agarose gel electrophoresis for the presence of either a wild-type fragment of 733 bp, or a truncated 325 bp product corresponding to the exon 15-17 deleted splice form. Both wild-type and exon 15-17 deleted transcripts were found in ovarian tissue of OV-HR2 and OV-CA4, and in the lymphocytes from both subjects, and in all samples of normal ovarian epithelial cells and lymphocytes analyzed. Sequencing of genomic DNA from both subjects from FANCD2 exon 14 to exon 19 revealed no mutations of the consensus splice sites. A protein encoded by the form lacking exons 15-17 was predicted to be 145 kDa. In addition, both transcripts were found in samples of normal ovarian epithelial cells and normal lymphocytes (FIG. 9A). Sequencing of genomic DNA from both patients from FANCD2 exons 15 to 20 revealed no mutations of the consensus splice sites. However, immunoblots of normal ovarian epithelial cells using an antibody targeted to the FANCD2 amino terminus did not reveal the presence of a protein of this molecular weight. Thus, although this spliced form is present in normal as well as cancer cells, it does not appear to give rise to a protein product.

The ability of this splice variant either to complement a FANCD2-deficient cell or to suppress activity of wild-type FANCD2 in abnormal cells was also explored. Ectopic expression of a FANCD2ex16-18del cDNA in the PD20 fibroblast line from a FANCD2-deficient Fanconi anemia patient did not correct the MMC hypersensitivity of these cells in contrast to expression of a wild-type FANCD2 construct (FIG. 9B). In addition, overexpression of the splice variant in the normal lymphoblast cell line JY had no effect on the survival of these cells after MMC exposure (FIG. 9C), suggesting that it does not act as a dominant-negative protein.

### Example 6

### Evaluation of FA Genes by Real-time RT-PCR

Expression levels of nucleic acids involved in protection from DNA damage were evaluated by RT-PCR to determine whether quantitative changes in expression correlated with increased sensitivity of cells to cross-linking agents.

Real-time RT-PCR was utilized to quantify transcripts of 23 genes that have previously been shown to play a role in protection against DNA damaging agents, such as MMC (Table 3). Using reverse-transcribed mRNA prepared from the high-risk OV-HR2 and cancer OV-CA4 primary cells, relative expression levels of these genes were measured using gene-specific TAQMAN® probes. Expression levels were normalized to an internal 18S ribosomal RNA control, compared to levels from two normal control samples. The relative expression was then calculated and expressed as fold change. Of the genes examined only FANCD2 was consistently lower in both subject samples compared to normal controls (6.4-fold lower in OV-HR2 and 5.0-fold lower in OV-CA4).

RNA and first-strand cDNA were prepared as described above. Real-time PCR was then performed on triplicate 50 ng aliquots of each cDNA sample using TAQMAN® Universal PCR Master Mix and an ABI PRISM® 7000 Sequence Detection System (Applied Biosystems), according to manufacturer instructions. All reactions were performed in multiplex format with a VIC/MGB-labeled, primer-limited eukaryotic 18S rRNA internal standard probe (Applied Biosystems). After PCR, threshold cycles were determined for each gene, and then values normalized using the threshold cycles of the 18S rRNA standard. The mean normalized value of each triplicate was determined, and fold change calculated using the delta-delta Cₜ method (Livak and Schmittgen, Methods 25:402-8, 2001), using the mean normalized value of two normal control samples as reference. Pre-designed primer and probe sets for 16 of the genes were purchased as TAQMAN® Gene Expression Assays from Applied Biosystems, and are as follows (assay ID in parentheses): ATR (Hs00169878_ml), BID (Hs00609630_ml), BLM (Hs00172060_ml), DCLRE1C (Hs00223928_ml), ERCC1 (Hs00157415), ERCC4 (Hs00193342_ml), H2AFX (Hs00266783_sl), HTATIP (Hs00197310_ml), MRE11A (Hs00271551_ml), NBN (Hs00159537_ml), RAD51 (Hs00153418_ml), RAD54L (Hs00269177_ml), REV3L (Hs00161301_ml), XRCC2 (Hs00538799_ml), XRCC3 (Hs00193725_ml), and FANCL (Hs01015742_ml). The remainder of the primer/probe sets was designed with the aid of ABI PRISM® Primer Express software v.2.0.0 (Applied Biosystems). Sequences are listed in Table 2. Primers for these sets were synthesized by Integrated DNA Technologies (Coralville, Iowa), while 6-FAM/MGB probes were made by Applied Biosystems.

TABLE 2
Primers designed to quantify FANC mRNA. All
sequences are written from 5′.

| Gene | | |
|---|---|---|
| Symbol | | Sequence |
| FANCA | Forward | AGCGGTGTGGCATCTTCAC |
| | primer | (SEQ ID NO: 13) |
| | Reverse | GCATGTCGGGATGGCTTTC |
| | primer | (SEQ ID NO: 14) |
| | Probe | CAAGGCATTGTGAGCCT |
| | | (SEQ ID NO: 15) |
| FANCC | Forward | GGAAATCCTCCAGCCAGAGTT |
| | primer | (SEQ ID NO: 16) |
| | Reverse | GGAGAGAAATCTTCTTCAGCAAAATG |
| | primer | (SEQ ID NO: 17) |
| | Probe | TGAGGCTGTAAACGAGG |
| | | (SEQ ID NO: 18) |
| FANCD2 | Forward | TGAAATGCACACTGAAGCTACAGA |
| | primer | (SEQ ID NO: 19) |
| | Reverse | GAGATCTTCCAGCAAGAAAAGCA |
| | primer | (SEQ ID NO: 20) |
| | Probe | CAACTTGGGCCCCCTG |
| | | (SEQ ID NO: 21) |
| FANCE | Forward | TCAGCCTCAGCAATGCTACTGT |
| | primer | (SEQ ID NO: 22) |
| | Reverse | AAGGAGAGGATCCGTCCAAGA |
| | primer | (SEQ ID NO: 23) |
| | Probe | CTGACCAGAAGCCTC |
| | | (SEQ ID NO: 24) |
| FANCF | Forward | CGTCGGCCCCAAGAAGA |
| | primer | (SEQ ID NO: 25) |
| | Reverse | TCCCCTCTCCAGGTGATTTG |
| | primer | (SEQ ID NO: 26) |
| | Probe | TGGAACCCGGCATC |
| | | (SEQ ID NO: 27) |
| FANCG | Forward | TGTCCTCCTGACAGCATTTGC |
| | primer | (SEQ ID NO: 28) |
| | Reverse | TGTCTGGGTTCCCTGTGATCA |
| | primer | (SEQ ID NO: 29) |
| | Probe | CGCCAAGGTCTCCAG |
| | | (SEQ ID NO: 30) |
| FANCM | Forward | TGCCAAGTGCGGGACTAC |
| | primer | (SEQ ID NO: 31) |
| | Reverse | TAGGCAGACACACCAGCGTATT |
| | primer | (SEQ ID NO: 32) |
| | Probe | CACATTTCCCGGGCTG |
| | | (SEQ ID NO: 33) |

TABLE 3
Relative mRNA levels of DNA repair and FA genes.

| Gene | | OV-HR2 | | OV-CA4 | |
|---|---|---|---|---|---|
| Symbol | Gene Name | Fold Change | Call | Fold Change | Call |
| ATR | ataxia | 1.9 (range 1.7-2.1) | UP | 2.1 (range 1.7-2.5) | DN |
| | telangiectasia, | | | | |
| | Rad3-related | | | | |
| BID | BH3 interacting | 1.5 (range 1.3-1.8) | UP | 2.7 (range 2.2-3.3) | DN |
| | domain death | | | | |
| | agonist | | | | |
| BLM | Bloom | 1.3 (range 1.1-1.5) | DN | 4.0 (range 3.6-4.5) | DN |
| | syndrome | | | | |
| BRCA2 | breast cancer 2, | 4.2 (range 2.3-7.7) | DN | 4.7 (range 3.0-7.3) | DN |
| (FANCD1) | early onset | | | | |
| DCLRE1C | DNA crosslink | 1.3 (range 1.3-1.4) | DN | 1.6 (range 1.4-1.8) | DN |
| | repair 1C | | | | |
| | (PSO2 | | | | |
| | homolog, S. cerevisiae) | | | | |
| ERCC1 | excision repair | 1.2 (range 1.1-1.3) | DN | 1.3 (range 0.9-1.9) | DN |
| | cross- | | | | |
| | complementing | | | | |
| | rodent repair | | | | |
| | deficiency 1 | | | | |
| ERCC4 | excision repair | 2.0 (range 1.3-2.8) | UP | 1.3 (range 1.1-1.5) | DN |
| | cross- | | | | |
| | complementing | | | | |
| | rodent repair | | | | |
| | deficiency 2 | | | | |
| H2AFX | H2A histone | 2.5 (range 2.0-3.2) | UP | 1.2 (range 1.0-1.5) | UP |
| | family, member X | | | | |
| HTATIP | HIV-1 Tat | 1.0 (range 0.9-1.1) | — | 1.1 (range 0.8-1.5) | — |
| | interactive | | | | |
| | protein, 60 kDa | | | | |
| MRE11A | mitotic | 1.0 (range 0.7-1.5) | — | 1.1 (range 0.9-1.3) | — |
| | recombination | | | | |
| | 11 | | | | |
| NBN | nibrin, p95 | 1.1 (range 1.0-1.2) | — | 1.0 (range 0.8-1.3) | — |
| | protein of | | | | |
| | MRE11/RAD5 | | | | |
| | 0 complex | | | | |
| RAD51 | RAD51 | 1.4 (range 1.0-1.8) | DN | 2.3 (range 1.8-3.0) | DN |
| | homolog | | | | |
| RAD54L | RAD54-like (S. cerevisiae) | 1.8 (range 1.5-2.1) | DN | 1.6 (range 1.3-1.9) | DN |
| REV3L | REV3-like, | 1.9 (range 1.5-2.3) | UP | 2.2 (range 1.5-3.1) | DN |
| | catalytic | | | | |
| | subunit of DNA | | | | |
| | polymerase zeta | | | | |
| | (yeast) | | | | |
| XRCC2 | X-ray repair | 1.1 (range 0.9-1.3) | — | 1.0 (range 0.6-1.8) | — |
| | complementing | | | | |
| | defective repair | | | | |
| | in CHO cells 2 | | | | |
| XRCC3 | X-ray repair | 1.5 (range 1.3-1.9) | DN | 1.1 (range 0.8-1.4) | — |
| | complementing | | | | |
| | defective repair | | | | |
| | in CHO cells 3 | | | | |
| FANCA | | 1.4 (range 1.1-1.9) | DN | 1.1 (range 0.9-1.3) | — |
| FANCC | | 1.0 (range 0.6-1.6) | — | 1.2 (range 0.7-2.3) | DN |
| FANCD2 | | 6.4 (range 4.6-6.9) | DN | 5.0 (range 4.1-6.6) | DN |
| FANCE | | 1.6 (range 1.1-1.8) | DN | 1.1 (range 1.0-1.2) | — |
| FANCF | | 1.9 (range 1.1-3.2) | UP | 1.2 (range 0.7-2.2) | Up |
| FANCG | | 1.4 (range 1.1-1.8) | DN | 3.1 (range 2.5-3.7) | DN |
| FANCJ | | 4.8 (range 4.5-5.2) | DN | 18.1 (range 13.8-23.8) | DN |
| FANCL | | 1.9 (range 1.8-2.1) | DN | 1.8 (range 1.1-3.0) | DN |
| FANCM | | 2.3 (range 2.2-2.3) | DN | 1.9 (range 1.5-2.4) | DN |

“UP” indicates an increase of 1.2-fold or more of the indicated mRNA in patient sample, compared to normal control.
“DN” indicates a decrease of 1.2-fold or more in patient sample, compared to normal control.
“—” indicates no difference in mRNA level between patient and normal control.

### Example 7

### Evaluation of DNA Copy Number of Genes Implicated in Chromosome Instability

This example illustrates array based techniques for monitoring the expression of FA NNC component nucleic acids.

Comparative genomic hybridization analysis on whole-genome oligonucleotide arrays was performed on samples OV-HR2 and OV-CA4 by the method of Selzer et al. (Selzer et al., Genes Chrom. Cancer 44:305-19, 2005). In both samples, the FANCD2 gene locus was intact, with no gain or loss of 3p25.3 sequences at the array CGH resolution that was tested (6 Kb median probe spacing, or twelve probes for the ˜75 Kb FANCD2 gene). Similarly, there were no amplifications or deletions of sequences of seven other FA genes or, fifteen DNA damage response and repair genes analyzed (Table 4). As expected, some other genomic losses were identified in these transformed cells (Table 5).

The oligonucleotide array comparative genomic hybridization (oa-CGH) method used here was described previously by Selzer et al. 2005 (Selzer et al., Genes Chrom. Cancer 44:305-19, 2005). Briefly, a whole-genome array with a 6 Kb median probe spacing was used to map single and multiple copy number genomic alterations. Oligonucleotide probes were of isothermal design (Tₘ=76° C.) and were tiled through genic and inter-genic regions. Probe lengths varied from 45 to 85 nucleotides. Genomic DNA samples extracted from primary ovarian epithelial cell cultures were labeled according to method of Selzer for 5 minutes and cooled to 42° C. Hybridizations were carried out for 18 hours at 42° C. (Selzer et al., Genes Chrom. Cancer 44:305-19, 2005). DNA was fragmented to 500-2000 bp by sonication, the DNA was heat-denatured, and then hybridized with random nonamers containing a 5′-Cy3 or 5′-Cy5 dye (TriLink Biotechnologies, San Diego, Calif.). Samples were chilled on ice and then incubated with 100 U Klenow fragment (NEW ENGLAND BIOLABS® Ipswich, Mass.) and 6 mM dNTP mix (INVITROGEN™) for 2 hours at 37° C. Reactions were terminated by adding 0.5M EDTA pH 8.0, the products were precipitated with isopropanol and resuspended in water. A typical amplification resulted in a 50-fold increase. Differentially labeled test and reference sample (15 μg of each) were combined and dried down. The reference sample was a pool of DNA (extracted from peripheral blood lymphocytes) from 6 male individuals (Promega, Madison, Wis.). The samples were rehydrated in NimbleGen Hybridization Buffer (NimbleGen Systems, Madison, Wis.) denatured at 95° C. The arrays were washed with NimbleGen Wash Buffer System and dried by centrifugation.

Arrays were scanned at 5 μm resolution using a GenePix 4000B scanner (Axon Instruments, Molecular Devices Corp., Sunnyvale, Calif.). Data were extracted from scanned images using NimbleScan 2.0 extraction software (NimbleGen Systems, Inc.). Data analysis included normalization of signal intensities of the test sample versus reference sample. The log₂ ratios were averaged with a fixed window size corresponding to 5×, 10×, and 20× the median probe spacing. Unaveraged and window-averaged log₂ ratios were used as input to the DNA copy package of the Bioconductor software to produce the final segmentations (Olshen et al., Biostat. 5:557-72, 2004) that demarcate DNA copy number changes.

TABLE 4
Genes analyzed by oa-CGH. No gene was found to
have deletions or amplifications of genomic sequences.

| FA genes | DNA repair/response genes |
|---|---|
| FANCA | RAD54L |
| FANCC | DCLRE1C |
| FANCD2 | H2AFX |
| FANCE | HTATIP |
| FANCF | MRE11A |
| FANCG | XRCC3 |
| FANCL | RAD51 |
| FANCM | BLM |

ERCC4
ERCC1
BID
ATR
REV3L
XRCC2
NBN

TABLE 5
Genomic losses identified by whole genomic oa-CGH.

| | Chromosome | DNA sequence | |
|---|---|---|---|
| Cell line | band | coordinate^{a} | Genes present^{b} |
| OV-HR2 | 1q32.1 | 199173282-199267956 | PPP1R12B |
| | 6p21.3 | 31479350-31479458 | MICA |
| | 9p22.3 | 14791818-14839785 | FREMI |
| | 22q11.1 | 18623365-18623937 | ACTBL1/POTE14 |
| | 14q11.2 | 19385491-19491463 | HLA-DRB |
| | 22q13.1 | 37686171-37712360 | GVDC^{c} |
| OV-CA4 | 6p21.3 | 32593507-32593134 | HLA-DRB5 |
| | 8q22.3 | 100094670-100094751 | VPS13B |
| | 11p15.2-14.3 | 1122646230-22646457 | GAS2 |
| | 5q13.2 | 69690000-69990000 | GVD |
| | 8p23.1 | 7230000-7890000 | GVD |
| | 14q11.2 | 18750000-19470000 | GVD |
| | 15q11.2 | 18330000-20250000 | GVD |

^{a}Breakpoint interval defined by whole-genome oaCGH.
^{b}Only characterized genes are listed.
^{c}Regions of loss listed as a copy number variant in the Database of Genomic Variants (GVD).

### Example 8

### Analysis of the Promoter Methylation State of FA Genes

This example illustrates the determination of the promoter methylation state of FA NNC component genes.

To determine whether epigenetic silencing by promoter methylation could account for low levels of FANCD2 protein, the FANCD2 promoter, as well as all other FA gene promoters, was analyzed by MS-MLPA (Olshen et al., Biostat. 5:557-72, 2004).

Probes to promoter CpG islands were designed to include a HhaI methylation-specific restriction site within the detected sequence. Upon digestion with HhaI, probes with a methylated recognition sequence generate a signal. If the CpG site is unmethylated, the genomic DNA/MS-MLPA probe complex is digested, preventing exponential amplification, and signal detection after fragment analysis. Two probes were designed for the promoters of FA genes FANCB, -C, -D1, -D2, -E, -J, -L, and -M. One probe each was used for FANCA and FANCG, and three probes were used for FANCF. Probes to detect methylated promoters by the method of MS-MLPA were designed as described previously (Olshen et al., Biostat. 5:557-72, 2004), except that the promoter sequences detected by these probes contain a recognition site for HhaI methylation-specific restriction enzyme. Probes that were targeted against the promoter regions of all the identified FA genes (FANCA, -B, -C, -D1, -D2, -E, -F, -G, -J, -L, and -M) were used. Each FA gene was represented by two MS-MLPA probes, except FANCA and FANCG (one probe each) and FANCF (three probes). The MLPA reagents were obtained from MRC-Holland, Amsterdam, Netherlands. Approximately 25 ng of genomic DNA in 5 μl of TE buffer [10 mM Tris-HCl (pH 8.5) and 1 mM EDTA] were denatured for 10 minutes at 98° C. SALSA MLPA buffer (1.5 μl) and MS-MLPA probes (1 fmol each and 1.5 μl volume) were then added and after incubation for 1 minute at 95° C., were allowed to hybridize to their respective targets for 16 hours at 60° C. After hybridization, the mixture was diluted at room temperature with H₂O and 3 μl Ligase buffer A to a final volume of 20 μl and then equally divided in two tubes. While at 49° C., a mixture of 0.25 μl Ligase-65 (MRC-Holland), 5 U HhaI (INVTROGEN™) and 1.5 μl Ligase buffer B in a total volume of 10 μl was added to one tube. The second tube was treated identically except that the HhaI enzyme was replaced with H₂O. Simultaneous ligation and digestion was then performed by incubation for 30 min at 49° C., followed by 5 minutes heat inactivation of the enzymes at 98° C. The ligation products were PCR amplified by the addition of 5 μl of this ligation mixture to 20 μl PCR mixture containing PCR buffer, dNTPs, SALSA polymerase and PCR primers (one unlabeled and one D4-labeled) at 60° C. as described by Schouten et al. (Schouten et al., Nucleic Acids Research, 2002; 30(12): e57). PCR products were run on an ABI PRISM 310 Genetic Analyzer (Applied Biosystems), and analyzed using GeneScan analysis software V.3.7 (Applied Biosystems).

No methylation of any of the FA pathway gene promoters was detected, indicating that epigenetic silencing of components of the FA pathway, and in particular of FANCD2, FANCD1, and FANCJ, was not responsible for decreased activity of the FA NNC component.

### Example 9

### Identification of Agents Increase the Activity of the Fanconi Anemia Non-Nuclear Component

This example describes the methods that can be used to identify compounds that increase the activity of the FA NNC component.

A library of natural products are obtained, for example from the Developmental Therapeutics Program NCI/NIH, and screened for their effect on the FA NNC component, for example by increasing the expression of one or more of FANCD1, FANCD2, and FANCJ.

Immortalized OV-CA4 cells are combined with serial dilutions of each compound 1 nM to 10 mM). The sample is incubated from between 10 minutes and 24 hours to assess the expression of FANCD2, FANCD1, and FANCJ. 1× SDS loading buffer is added to the cells. After incubation at 95° C. for 10 min, samples are resolved onto polyacrylamide gel and transferred onto a PVDF membrane. Blots are probed with primary rabbit polyclonal antibodies specific to FANCD2, FANCD2, and FANCJ to assess expression relative to a control sample not treated with the agents. Alternatively, the cells are screened for decreases in the number of radials and/or chromosomal breakages formed. Agents that increase the activity of the FA NNC component are selected for further evaluation.

Potential therapeutic agents identified with these or other approaches, including the specific assays and screening systems described herein, are used as lead compounds to identify other agents having even greater modulatory effects on the FA NNC component. For example, chemical analogs of identified chemical entities, or variant, fragments of fusions of peptide agents, are tested for their activity in the assays described herein. Candidate agents also can be tested in cell lines and animal models of cancer and/or Fanconi anemia to determine their therapeutic value. The agents also can be tested for safety in animals, and then used for clinical trials in animals or humans.

In view of the many possible embodiments to which the principles of our invention may be applied, it should be recognized that the illustrated embodiment is only a preferred example of the invention and should not be taken as a limitation on the scope of the invention. Rather, the scope of the invention is defined by the following claims. We therefore claim as our invention all that comes within the scope and spirit of these claims.

1. A method for diagnosing one or more of ovarian cancer and breast cancer, the method comprising:
selecting a subject; and
detecting a decrease in activity of the FA NNC component in a portion of female reproductive tissue obtained from the subject relative to a control, wherein detecting the decrease in activity of the FA NNC component indicates a diagnosis of one or more of ovarian cancer or breast cancer in the subject.

2. The method of claim 1, wherein diagnosing comprises diagnosing one or both of an existing ovarian cancer or breast cancer, or a predisposition to developing one or both of ovarian cancer and breast cancer.

3. (canceled)

4. The method of claim 1, wherein selecting the subject comprises selecting a subject with at least one ovarian cancer or breast cancer risk factor.

5. The method of claim 4, wherein selecting the subject further comprises selecting a subject without a mutation in the BRCA1 or BRCA2 gene that is known to be associated with cancer.

6. The method of claim 1, wherein the at least one ovarian or breast cancer risk factor comprises prior diagnosis of existing breast cancer or ovarian cancer in the subject, a family history of one or more of breast cancer and ovarian cancer, or a combination thereof.

7. (canceled)

8. The method of claim 6, wherein the family history of one or more of breast or ovarian cancer comprises:
(a) prior ovarian cancer in one or more 1st degree relative(s);
(b) prior ovarian cancer in one or more 1st degree relative(s) before age 50;
(c) prior ovarian cancer in one or more 1st degree relative(s) and prior breast or ovarian cancer in one or more 1st or 2nd degree relative(s);
(d) prior breast or ovarian cancer in the subject and prior breast or ovarian cancer in one or more 1st or 2nd degree relative(s); or
(e) a combination thereof.

9. The method of claim 1, wherein the control comprises at least one control cell, or a statistical control.

10. The method of claim 9, wherein the at least one control cell comprises one or more of an immortalized ovarian epithelial cell, an ovarian cell from a subject without ovarian cancer, a cell from a subject without a risk factor for ovarian cancer, a cell of an ovarian tissue from the subject at an earlier time point, or a combination thereof.

11. (canceled)

12. The method of claim 1, wherein detecting the decrease in the activity of the FA NNC component comprises detecting a decrease in a biological function of the FA NNC component.

13. The method of claim 12, wherein detecting the decrease in the biological function of the FA NNC component comprises:
providing at least one cell of the female reproductive tissue from the subject;
contacting the at least one cell of the female reproductive tissue with at least one DNA crosslinking agent; and
detecting an increase in one or more of chromosomal breakage and radial formation in the at least one cell relative to the control, wherein an increase in one or more of chromosomal breakage and radial formation relative to the control indicates the subject has one or more of ovarian cancer and breast cancer or a predisposition to developing one or more of ovarian and breast cancer.

14. The method of claim 13, wherein the DNA crosslinking agent comprises an alkylating agent.

15. (canceled)

16. The method of claim 1, wherein detecting the decrease in activity of the FA NNC component comprises:
providing at least one cell of the female reproductive tissue from the subject; and
detecting a decrease in expression of at least one of FANCJ, FANCD1, or FANCD2 gene product in the at least one cell relative to the control, where a decrease in expression of at least one of the FANCJ, FANCD1, or FANCD2 gene product relative to the control indicates the subject has one or more of ovarian cancer and breast cancer or a predisposition to developing one or more of ovarian and breast cancer.

17. The method of claim 16, wherein the FANCJ, FANCD1, or FANCD2 gene product comprises a FANCJ, FANCD1, or FANCD2 nucleic acid.

18. The method of claim 17, comprising detecting a decrease in expression of the FANCJ, FANCD1, or FANCD2 nucleic acid by providing a sample of nucleic acids from the at least one cell and detecting the decrease in expression of the FANCJ, FANCD1, or FANCD2 nucleic acid in a nucleic acid hybridization assay, a quantitative or semi-quantitative amplification assay, or a combination thereof.

19. The method of claim 18, wherein detecting the decrease in expression of the FANCJ, FANCD1, or FANCD2 nucleic acid comprises contacting the sample of nucleic acids from the at least one cell with a target nucleic acid comprising a nucleotide sequence that hybridizes to the FANCJ, FANCD1, or FANCD2 nucleic acid.

20. The method of claim 19, wherein the target nucleic acid comprises a nucleotide sequence that hybridizes to SEQ ID NO: 1 under high stringency conditions, a nucleotide sequence that hybridizes to SEQ ID NO: 3 under high stringency conditions, a nucleotide sequence that hybridizes to SEQ ID NO: 5 under high stringency conditions, or a nucleotide sequence that hybridizes to SEQ ID NO: 7 under high stringency conditions.

21. The method of claim 19, wherein the target nucleic acid comprises a microarray.

22. (canceled)

23. The method of claim 18, wherein the amplification assay comprises an RT-PCR assay.

24. The method of claim 18, wherein the amplification assay is performed with at least one primer comprising a nucleotide sequence that hybridizes to SEQ ID NO: 1 under high stringency conditions, a nucleotide sequence that hybridizes to SEQ ID NO: 3 under high stringency conditions, a nucleotide sequence that hybridizes to SEQ ID NO: 5 under high stringency conditions, or a nucleotide sequence that hybridizes to SEQ ID NO: 7 under high stringency conditions.

25. The method of claim 16, wherein the FANCJ, FANCD1, or FANCD2 gene product comprises a FANCJ, FANCD1, or FANCD2 protein.

26. The method of claim 25, wherein the decrease in expression of the FANCJ, FANCD1, or FANCD2 protein is detected by one or more of an immunohistochemical assay, a radioimmunoassay, a Western blot assay, an immunofluorescent assay, an enzyme immunoassasy, chemiluminescent assay, or mass spectrometry.

27.-29. (canceled)

30. The method of claim 1, wherein the female reproductive tissue comprises breast or ovarian tissue.

31. The method of claim 30, wherein the ovarian tissue comprises ovarian epithelial tissue, an ovarian brushing sample or a combination thereof.

32. (canceled)

33. The method of claim 1, wherein the female reproductive tissue comprises cervical tissue.

34. The method of claim 33, wherein the cervical tissue comprises cervical epithelial tissue.

35. The method of claim 33, wherein the cervical tissue comprises a PAP smear sample.

36. The method of claim 1, wherein obtaining the female reproductive tissue comprises a biopsy.

37. The method of claim 1, comprising detecting the activity of the FA NNC component in the female reproductive tissue of the subject following administration of an anti-neoplastic agent.

38. (canceled)

39. The method of claim 37, wherein the activity of the FA NNC component in female reproductive tissue obtained at a first time point is compared to the activity the FA NNC component in female reproductive tissue obtained at a second time point.

40. A method for monitoring a response of a subject to a therapy for treatment of a breast or ovarian tumor, the method comprising:
selecting a subject; and
detecting the activity of the FA NNC component in a portion of female reproductive tissue of the subject following administration of the therapy, wherein a decrease in the activity of the FA NNC component indicates an undesired response to the therapy and an increase in the activity of the FA NNC component indicates a desired response to the therapy.

41. (canceled)

42. The method of claim 40, wherein the activity of the FA NNC component in female reproductive tissue obtained at a first time point is compared to the activity the FA NNC component in female reproductive tissue obtained at a second later time point.

43. A method for identifying an agent that inhibits ovarian cancer or breast cancer, the method comprising:
contacting at least one cell with a test agent; and
detecting an increase in activity of the FA NNC component relative to a control, wherein an increase in the activity of the FA NNC component relative to the control identifies the agent as one that inhibits ovarian cancer or breast cancer.

44. The method of claim 43, wherein the cell is an ovarian cancer cell.

45. The method of claim 43, wherein the agent is a chemical compound, a small molecule, an antibody, or an antisense nucleic acid.

46. The method of claim 43, wherein the method comprises a high throughput technique.

47. The method of claim 43, wherein the control is a standard value.

48. The method of claim 43, wherein the control comprises a cell not contacted with the agent.

49. The method of claim 43, wherein detecting the increase in the activity of the FA NNC component comprises
contacting the at least one cell with at least one DNA crosslinking agent; and
detecting a decrease in one or more of chromosomal breakage and radial formation in the at least one cell relative to the control.

50. The method of claim 49, where the DNA crosslinking agent comprises an alkylating agent.

51. (canceled)

52. The method of claim 43, wherein detecting the increase in activity of the FA NNC component comprises:
detecting an increase in expression of at least one of FANCJ, FANCD1, or FANCD2 gene product in the at least one cell relative to the control.

53. The method of claim 52, wherein the FANCJ, FANCD1, or FANCD2 gene product comprises a FANCJ, FANCD1, or FANCD2 nucleic acid.

54. The method of claim 53, comprising detecting a decrease in expression of the FANCD2, FANCD1, or FANCJ nucleic acid by providing a sample of nucleic acids from the at least one cell and detecting the decrease in expression of FANCJ, FANCD1, or FANCD2 nucleic acid in a nucleic acid hybridization assay, a quantitative or semi-quantitative amplification assay, or a combination thereof.

55. The method of claim 54, wherein detecting the decrease in expression of the FANCJ, FANCD1, or FANCD2 nucleic acid comprises contacting the sample of nucleic acids from the at least one cell with a target nucleic acid comprising a nucleotide sequence that hybridizes to a FANCJ, FANCD1, or FANCD2 nucleic acid.

56. The method of claim 55, wherein the target nucleic acid comprises a nucleotide sequence that hybridizes to SEQ ID NO: 1 under high stringency conditions, a nucleotide sequence that hybridizes to SEQ ID NO: 3 under high stringency conditions, a nucleotide sequence that hybridizes to SEQ ID NO: 5 under high stringency conditions, or a nucleotide sequence that hybridizes to SEQ ID NO: 7 under high stringency conditions.

57. The method of claim 55, wherein the target nucleic acid comprises a microarray.

58. (canceled)

59. The method of claim 54, wherein the amplification assay comprises an RT-PCR assay.

60. The method of claim 54, wherein the amplification assay is performed with at least one primer comprising a nucleotide sequence that hybridizes to SEQ ID NO: 1 under high stringency conditions, a nucleotide sequence that hybridizes to SEQ ID NO: 3 under high stringency conditions, a nucleotide sequence that hybridizes to SEQ ID NO: 5 under high stringency conditions, or a nucleotide sequence that hybridizes to SEQ ID NO: 7 under high stringency conditions.

61. The method of claim 55, wherein the FANCJ, FANCD1, or FANCD2 gene product comprises a FANCJ, FANCD1, or FANCD2 protein.

62. The method of claim 61, wherein the decrease in expression of the FANCJ, FANCD1, or FANCD2 protein is detected by one or more of an immunohistochemical assay, a radioimmunoassay, a Western blot assay, an immunofluorescent assay, an enzyme immunoassasy, a chemiluminescent assay, or mass spectrometry.

63. (canceled)
